(19) United States
(12) Patent Application Publication
(10) Pub. No.: US 2021/0085180 A1
(43) Pub. Date: Mar. 25, 2021

(54) Computational User-Health Testing

(71) Applicant: WINTERLIGHT LABS INC., Toronto (CA)

(72) Inventors: Edward K.Y. Jung, Bellevue, WA (US); Eric C. Leuthardt, St. Louis, MO (US); Royce A. Levien, Cambridge, MA (US); Robert W. Lord, Seattle, WA (US); Mark A. Malamud, Seattle, WA (US)

(21) Appl. No.: 16/916,745

(22) Filed: Jun. 30, 2020

Related U.S. Application Data
(63) Continuation of application No. 12/156,433, filed on May 29, 2008, now abandoned.
Application No. 12/156,433 is a continuation-in-part of application No. 12/154,279, filed on May 20, 2008.
Application No. 12/154,279 is a continuation-in-part of application No. 12/151,742, filed on May 7, 2008, now abandoned.
Application No. 12/151,742 is a continuation-in-part of application No. 11/811,865, filed on Jun. 11, 2007, now abandoned.
Application No. 11/811,865 is a continuation-in-part of application No. 11/804,304, filed on May 15, 2007, now abandoned.
Application No. 11/804,304 is a continuation-in-part of application No. 11/731,745, filed on Mar. 30, 2007, now abandoned.
Application No. 11/731,745 is a continuation-in-part of application No. 11/731,778, filed on Mar. 30, 2007, now abandoned.
Application No. 11/731,778 is a continuation-in-part of application No. 11/731,801, filed on Mar. 30, 2007, now abandoned.

(51) Int. Cl. A61B 5/00; A61B 5/16; G16H 40/67

(52) U.S. Cl. CPC A61B 5/0022; A61B 5/162; A61B 5/4076; A61B 5/4023; A61B 5/4064; G16H 40/67; A61B 5/4082; A61B 5/4088

(57) ABSTRACT

Methods, apparatuses, computer program products, devices and systems are described that carry out accepting user brain activity measurement data; selecting at least one user-health test function at least partly based on the user brain activity measurement data; and applying the at least one user-health test function to at least one interaction between at least one user and at least one device-implemented application.

### CROSS-REFERENCE TO RELATED APPLICATIONS

The present application is related to and claims the benefit of the earliest available effective filing date(s) from the following listed application(s) (the “Related Applications”) (e.g., claims earliest available priority dates for other than provisional patent applications or claims benefits under 35 USC § 119(e) for provisional patent applications, for any and all parent, grandparent, great-grandparent, etc. applications of the Related Application(s)).

### RELATED APPLICATIONS

For purposes of the USPTO extra-statutory requirements, the present application constitutes a continuation-in-part of United States Patent Application No. [Attorney Docket No. 0406-002-005-CIP001], entitled COMPUTATIONAL USER-HEALTH TESTING, naming Edward K. Y. Jung; Eric C. Leuthardt; Royce A. Levien; Robert W. Lord; and Mark A. Malamud as inventors, filed 20 May 2008 which is currently co-pending, or is an application of which a currently co-pending application is entitled to the benefit of the filing date.
For purposes of the USPTO extra-statutory requirements, the present application constitutes a continuation-in-part of U.S. patent application Ser. No. 12/151,742, entitled COMPUTATIONAL USER-HEALTH TESTING, naming Edward K. Y. Jung; Eric C. Leuthardt; Royce A. Levien; Robert W. Lord; and Mark A. Malamud as inventors, filed 7 May 2008 which is currently co-pending, or is an application of which a currently co-pending application is entitled to the benefit of the filing date.
For purposes of the USPTO extra-statutory requirements, the present application constitutes a continuation-in-part of U.S. patent application Ser. No. 11/811,865, entitled COMPUTATIONAL USER-HEALTH TESTING, naming Edward K. Y. Jung; Eric C. Leuthardt; Royce A. Levien; Robert W. Lord; and Mark A. Malamud as inventors, filed 11 Jun. 2007 which is currently co-pending, or is an application of which a currently co-pending application is entitled to the benefit of the filing date.
For purposes of the USPTO extra-statutory requirements, the present application constitutes a continuation-in-part of U.S. patent application Ser. No. 11/804,304, entitled COMPUTATIONAL USER-HEALTH TESTING, naming Edward K. Y. Jung; Eric C. Leuthardt; Royce A. Levien; Robert W. Lord; and Mark A. Malamud as inventors, filed 15 May 2007 which is currently co-pending, or is an application of which a currently co-pending application is entitled to the benefit of the filing date.
For purposes of the USPTO extra-statutory requirements, the present application constitutes a continuation-in-part of U.S. patent application Ser. No. 11/731,745, entitled EFFECTIVE RESPONSE PROTOCOLS FOR HEALTH MONITORING OR THE LIKE, naming Edward K. Y. Jung; Eric C. Leuthardt; Royce A. Levien; Robert W. Lord; and Mark A. Malamud as inventors, filed 30 Mar. 2007 which is currently co-pending, or is an application of which a currently co-pending application is entitled to the benefit of the filing date.
For purposes of the USPTO extra-statutory requirements, the present application constitutes a continuation-in-part of U.S. patent application Ser. No. 11/731,778, entitled CONFIGURING SOFTWARE FOR EFFECTIVE HEALTH MONITORING OR THE LIKE, naming Edward K. Y. Jung; Eric C. Leuthardt; Royce A. Levien; Robert W. Lord; and Mark A. Malamud as inventors, filed 30 Mar. 2007 which is currently co-pending, or is an application of which a currently co-pending application is entitled to the benefit of the filing date.
For purposes of the USPTO extra-statutory requirements, the present application constitutes a continuation-in-part of U.S. patent application Ser. No. 11/731,801, entitled EFFECTIVE LOW PROFILE HEALTH MONITORING OR THE LIKE, naming Edward K. Y. Jung; Eric C. Leuthardt; Royce A. Levien; Robert W. Lord; and Mark A. Malamud as inventors, filed 30 Mar. 2007 which is currently co-pending, or is an application of which a currently co-pending application is entitled to the benefit of the filing date.

The United States Patent Office (USPTO) has published a notice to the effect that the USPTO's computer programs require that patent applicants reference both a serial number and indicate whether an application is a continuation or continuation-in-part. Stephen G. Kunin, Benefit of Prior Filed Application, USPTO Official Gazette Mar. 18, 2003, available at http://www.uspto.gov/web/offices/com/sol/og/2003/weekll/patbene.htm. The present Applicant Entity (hereinafter “Applicant”) has provided above a specific reference to the application(s) from which priority is being claimed as recited by statute. Applicant understands that the statute is unambiguous in its specific reference language and does not require either a serial number or any characterization, such as “continuation” or “continuation-in-part,” for claiming priority to U.S. patent applications. Notwithstanding the foregoing, Applicant understands that the USPTO's computer programs have certain data entry requirements, and hence Applicant is designating the present application as a continuation-in-part of its parent applications as set forth above, but expressly points out that such designations are not to be construed in any way as any type of commentary and/or admission as to whether or not the present application contains any new matter in addition to the matter of its parent application(s).

All subject matter of the Related Applications and of any and all parent, grandparent, great-grandparent, etc. applications of the Related Applications is incorporated herein by reference to the extent such subject matter is not inconsistent herewith.

### TECHNICAL FIELD

This description relates to data capture and data handling techniques.

### SUMMARY

An embodiment provides a method. In one aspect, a method includes but is not limited to accepting user brain activity measurement data; selecting at least one user-health test function at least partly based on the user brain activity measurement data; and applying the at least one user-health test function to at least one interaction between at least one user and at least one device-implemented application. In addition to the foregoing, other method aspects are described in the claims, drawings, and text forming a part of the present disclosure.

In one or more various aspects, related systems include but are not limited to circuitry and/or programming for effecting the herein-referenced method aspects; the circuitry and/or programming can be virtually any combination of hardware, software, and/or firmware configured to effect the herein-referenced method aspects depending upon the design choices of the system designer.

An embodiment provides a computer program product. In one aspect, the computer program product includes but is not limited to a signal-bearing medium bearing (a) one or more instructions for accepting user brain activity measurement data; (b) one or more instructions for selecting at least one user-health test function at least partly based on the user brain activity measurement data; and (c) one or more instructions for applying the at least one user-health test function to at least one interaction between at least one user and at least one device-implemented application. In addition to the foregoing, other computer program product aspects are described in the claims, drawings, and text forming a part of the present disclosure.

An embodiment provides a system. In one implementation, the system includes but is not limited to a computing device and instructions. The instructions when executed on the computing device cause the computing device to (a) accept user brain activity measurement data; (b) select at least one user-health test function at least partly based on the user brain activity measurement data; and (c) apply the at least one user-health test function to at least one interaction between at least one user and at least one device-implemented application. In addition to the foregoing, other system aspects are described in the claims, drawings, and text forming a part of the present disclosure.

In one or more various aspects, related systems include but are not limited to computing means and/or programming for effecting the herein-referenced system aspects; the computing means and/or programming may be virtually any combination of hardware, software, and/or firmware configured to effect the herein-referenced system aspects depending upon the design choices of the system designer.

In addition to the foregoing, various other method and/or system and/or program product aspects are set forth and described in the teachings such as text (e.g., claims and/or detailed description) and/or drawings of the present disclosure.

The foregoing is a summary and thus may contain simplifications, generalizations, inclusions, and/or omissions of detail; consequently, those skilled in the art will appreciate that the summary is illustrative only and is NOT intended to be in any way limiting. Other aspects, features, and advantages of the devices and/or processes and/or other subject matter described herein will become apparent in the teachings set forth herein.

### BRIEF DESCRIPTION OF THE DRAWINGS

With reference now to FIG. 1, shown is an example of a user interaction and data processing system in which embodiments may be implemented, perhaps in a device, which may serve as a context for introducing one or more processes and/or devices described herein.

FIG. 2 illustrates certain alternative embodiments of the data capture and processing system of FIG. 1.

FIG. 3 illustrates certain alternative embodiments of the data capture and processing system of FIG. 1.

With reference now to FIG. 3, shown is an example of an operational flow representing example operations related to computational user-health testing, which may serve as a context for introducing one or more processes and/or devices described herein.

FIG. 4 illustrates an alternative embodiment of the example operational flow of FIG. 3.

FIG. 5 illustrates an alternative embodiment of the example operational flow of FIG. 3.

FIG. 7 illustrates an alternative embodiment of the example operational flow of FIG. 3.

FIG. 8 illustrates an alternative embodiment of the example operational flow of FIG. 3.

FIG. 9 illustrates an alternative embodiment of the example operational flow of FIG. 3.

FIG. 10 illustrates an alternative embodiment of the example operational flow of FIG. 3.

With reference now to FIG. 11, shown is a partial view of an example computer program product that includes a computer program for executing a computer process on a computing device related to computational user-health testing, which may serve as a context for introducing one or more processes and/or devices described herein.

With reference now to FIG. 12, shown is an example device in which embodiments may be implemented related to computational user-health testing, which may serve as a context for introducing one or more processes and/or devices described herein.

The use of the same symbols in different drawings typically indicates similar or identical items.

### DETAILED DESCRIPTION

FIG. 1 illustrates an example system 100 in which embodiments may be implemented. The system 100 includes at least one device 102. The at least one device 102 may contain, for example, an application 104 and a user-health test function unit 140. User-health test function unit 140 may generate user-health data 116, or user-health data 116 may be obtained from a user-health data module 150 that is external to the at least one device 102.

User-health test function unit 140 may include user health test function set 196, user health test function set 197, and/or user health test function set 198. The at least one device 102 may optionally include a data detection module 114, a data capture module 136, and/or a user-health test function selection module 138. The system 100 may also include a user input device 180, and/or a user monitoring device 182.

In some embodiments the user-health test function unit 140 and/or user-health test function selection module 138 may be located on an external device 194 that can communicate with the at least one device 102, on which the application 104 is operable, via network 192. In some embodiments, the application 104 may be located on an external device 194, and operable on the device 102 remotely via, for example, network 192.

In some embodiments, the user-health test function unit 140 may exist within the application 104. In other embodiments, the user-health test function unit 140 may be structurally distinct from the application 104.

User 190 may interact with application 104 through user interface 184. Further, user 190 may be monitored by brain activity measurement unit 186, which may be separate from device 102 or integrated in device 102.

In FIG. 1, the at least one device 102 is illustrated as possibly being included within a system 100. Of course, virtually any kind of computing device may be used in connection with the application 104, such as, for example, a workstation, a desktop computer, a mobile computer, a networked computer, a collection of servers and/or databases, cellular phone, personal entertainment device, or a tablet PC.

Additionally, not all of the application 104, user-health test function unit 140, and/or user-health test function selection module 138 need be implemented on a single computing device. For example, the application 104 may be implemented and/or operable on a remote computer, while the user interface 184 and/or user-health data 116 are implemented and/or stored on a local computer as the at least one device 102. Further, aspects of the application 104, user-health test function unit 140 and/or user-health test function selection module 138 may be implemented in different combinations and implementations than that shown in FIG. 1. For example, functionality of the user interface 184 may be incorporated into the at least one device 102. The at least one device 102, user-health test function unit 140, and/or user-health test function selection module 138 may perform simple data relay functions and/or complex data analysis, including, for example, fuzzy logic and/or traditional logic steps. Further, many methods of searching databases known in the art may be used, including, for example, unsupervised pattern discovery methods, coincidence detection methods, and/or entity relationship modeling. In some embodiments, the at least one device 102, user-health test function unit 140, and/or user-health test function selection module 138 may process user-health data 116 according to health profiles available as updates through a network.

The user-health data 116 may be stored in virtually any type of memory that is able to store and/or provide access to information in, for example, a one-to-many, many-to-one, and/or many-to-many relationship. Such a memory may include, for example, a relational database and/or an object-oriented database, examples of which are provided in more detail herein.

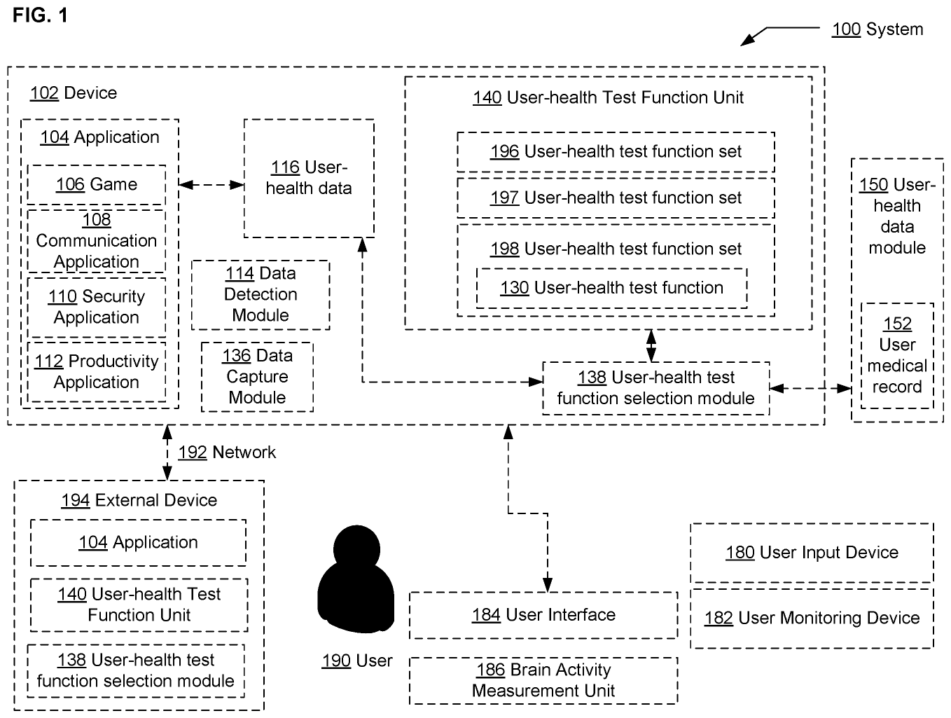

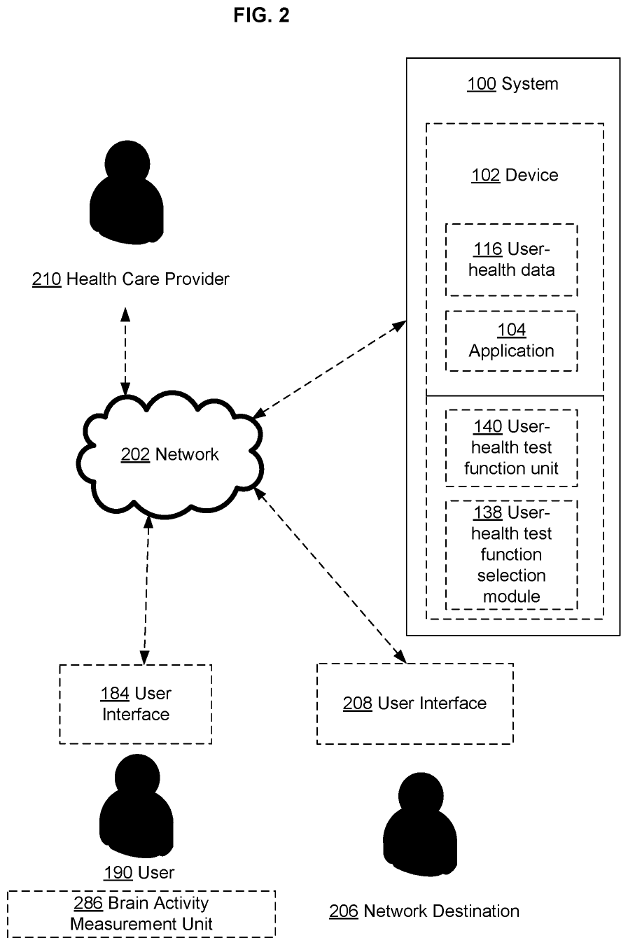

FIG. 2 illustrates certain alternative embodiments of the system 100 of FIG. 1. In FIG. 2, the user 190 may use the user interface 184 to interact through a network 202 with the application 104 operable on the at least one device 102. A user-health test function unit 140 and/or user-health test function selection module 138 may be implemented on the at least one device 102, or elsewhere within the system 100 but separate from the at least one device 102. The at least one device 102 may be in communication over a network 202 with a network destination 206 and/or healthcare provider 210, which may interact with the at least one device 102, user-health test function unit 140, and/or user-health test function selection module 138 through, for example, a user interface 208. Of course, it should be understood that there may be many users other than the specifically-illustrated user 190, for example, each with access to a local instance of the application 104.

In this way, the user 190, who may be using a device that is connected through a network 202 with the system 100 (e.g., in an office, outdoors and/or in a public environment), may generate user-health data 116 as if the user 190 were interacting locally with the at least one device 102 on which the application 104 is locally operable.

User 190 may interact with application 104 through user interface 184. Further, user 190 may be monitored by brain activity measurement unit 286, which may be separate from device 102 and/or integrated in device 102.

As referenced herein, the at least one device 102 and/or user-health test function selection module 138 may be used to perform various data querying and/or recall techniques with respect to the user-health data 116, in order to select at least one user-health test function at least partly based on the user-health data 116. For example, where the user-health data 116 is organized, keyed to, and/or otherwise accessible using one or more reference health condition attributes or profiles, various Boolean, statistical, and/or semi-boolean searching techniques may be performed to match user-health data 116 with reference health condition data, attributes, or profiles.

Many examples of databases and database structures may be used in connection with the at least one device 102, user-health test function unit 140, and/or user-health test function selection module 138. Such examples include hierarchical models (in which data is organized in a tree and/or parent-child node structure), network models (based on set theory, and in which multi-parent structures per child node are supported), or object/relational models (combining the relational model with the object-oriented model).

Still other examples include various types of eXtensible Mark-up Language (XML) databases. For example, a database may be included that holds data in some format other than XML, but that is associated with an XML interface for accessing the database using XML. As another example, a database may store XML data directly. Additionally, or alternatively, virtually any semi-structured database may be used, so that context may be provided to/associated with stored data elements (either encoded with the data elements, or encoded externally to the data elements), so that data storage and/or access may be facilitated.

Such databases, and/or other memory storage techniques, may be written and/or implemented using various programming or coding languages. For example, object-oriented database management systems may be written in programming languages such as, for example, C++ or Java. Relational and/or object/relational models may make use of database languages, such as, for example, the structured query language (SQL), which may be used, for example, for interactive queries for information and/or for gathering and/or compiling data from the relational database(s).

For example, SQL or SQL-like operations over one or more reference health conditions may be performed, or Boolean operations using a reference health condition may be performed. For example, weighted Boolean operations may be performed in which different weights or priorities are assigned to one or more of the reference health conditions, perhaps relative to one another. For example, a number-weighted, exclusive-OR operation may be performed to request specific weightings of desired (or undesired) health reference data to be included or excluded.

Accepting user brain activity measurement data of a user 190 may include measuring magnetic, electrical, hemodynamic, and/or metabolic activity in the brain.

Magnetoencephalography

One method of accepting user brain activity measurement data may include measuring the magnetic fields produced by electrical activity in the brain via magnetoencephalography (MEG) using magnetometers such as superconducting quantum interference devices (SQUIDs) or other devices. Such measurements are commonly used in both research and clinical settings to, e.g., assist researchers in determining the function of various parts of the brain. Synchronized neuronal currents induce very weak magnetic fields that can be measured by magnetoencephalography. However, the magnetic field of the brain is considerably smaller at 10 femtotesla (fT) for cortical activity and 103 fT for the human alpha rhythm than the ambient magnetic noise in an urban environment, which is on the order of 108 fT. Two essential problems of biomagnetism arise: weakness of the signal and strength of the competing environmental noise. The development of extremely sensitive measurement devices such as SQUIDs facilitates analysis of the brain's magnetic field in spite of the relatively low signal versus ambient magnetic signal noise. Magnetoencephalography (and EEG) signals derive from the net effect of ionic currents flowing in the dendrites of neurons during synaptic transmission. In accordance with Maxwell's equations, any electrical current will produce an orthogonally oriented magnetic field. It is this field that is measured with MEG. The net currents can be thought of as current dipoles, which are currents having an associated position, orientation, and magnitude, but no spatial extent. According to the right-hand rule, a current dipole gives rise to a magnetic field that flows around the axis of its vector component.

In order to generate a detectable signal, approximately 50,000 active neurons are needed. Because current dipoles must have similar orientations to generate magnetic fields that reinforce each other, it is often the layer of pyramidal cells in the cortex, which are generally perpendicular to its surface, that give rise to measurable magnetic fields. Further, it is often bundles of these neurons located in the sulci of the cortex with orientations parallel to the surface of the head that project measurable portions of their magnetic fields outside of the head.

Smaller magnetometers are in development, including a mini-magnetometer that uses a single milliwatt infrared laser to excite rubidium in the context of an applied perpendicular magnetic field. The amount of laser light absorbed by the rubidium atoms varies predictably with the magnetic field, providing a reference scale for measuring the field. The stronger the magnetic field, the more light is absorbed. Such a system is currently sensitive to the 70 fT range, and is expected to increase in sensitivity to the 10 fT range. See Physorg.com, “New mini-sensor may have biomedical and security applications,” Nov. 1, 2007, http://www.physorg.com/news113151078.html.

Electroencephalography

Another method of accepting user brain activity measurement data may include measuring the electrical activity of the brain by recording from electrodes placed on the scalp or, in special cases, subdurally, or in the cerebral cortex. The resulting traces are known as an electroencephalogram (EEG) and represent a summation of post-synaptic potentials from a large number of neurons. EEG is most sensitive to a particular set of post-synaptic potentials: those which are generated in superficial layers of the cortex, on the crests of gyri directly abutting the skull and radial to the skull. Dendrites that are deeper in the cortex, inside sulci, are in midline or deep structures (such as the cingulate gyms or hippocampus) or that produce currents that are tangential to the skull make a smaller contribution to the EEG signal.

One application of EEG is event-related potential (ERP) analysis. An ERP is any measured brain response that is directly the result of a thought or perception. ERPs can be reliably measured using electroencephalography (EEG), a procedure that measures electrical activity of the brain, typically through the skull and scalp. As the EEG reflects thousands of simultaneously ongoing brain processes, the brain response to a certain stimulus or event of interest is usually not visible in the EEG. One of the most robust features of the ERP response is a response to unpredictable stimuli. This response is known as the P300 (P3) and manifests as a positive deflection in voltage approximately 300 milliseconds after the stimulus is presented.

The most robust ERPs are seen after many dozens or hundreds of individual presentations are averaged together. This technique cancels out noise in the data allowing only the voltage response to the stimulus to stand out clearly. While evoked potentials reflect the processing of the physical stimulus, event-related potentials are caused by higher processes, such as memory, expectation, attention, or other changes in mental state.

A two-channel wireless brain wave monitoring system powered by a thermo-electric generator has been developed by IMEC (Interuniversity Microelectronics Centre, Leuven, Belgium). This device uses the body heat dissipated naturally from the forehead as a means to generate its electrical power. The wearable EEG system operates autonomously with no need to change or recharge batteries. The EEG monitor prototype is wearable and integrated into a headband where it consumes 0.8 milliwatts. A digital signal processing block encodes extracted EEG data, which is sent to a PC via a 2.4-GHz wireless radio link. The thermoelectric generator is mounted on the forehead and converts the heat flow between the skin and air into electrical power. The generator is composed of 10 thermoelectric units interconnected in a flexible way. At room temperature, the generated power is about 2 to 2.5-mW or 0.03-mW per square centimeter, which is the theoretical limit of power generation from the human skin. Such a device is proposed to associate emotion with EEG signals. See Clarke, “IMEC has a brain wave: feed EEG emotion back into games,” EE Times online, http://www.eetimes.eu/design/202801063 (Nov. 1, 2007).

EEG can be recorded at the same time as MEG so that data from these complimentary high-time-resolution techniques can be combined.

Accepting user brain activity measurement data of a member of population cohort 102 may also include measuring metabolic or hemodynamic responses to neural activity. For example, in positron emission tomography (PET), positrons, the antiparticles of electrons, are emitted by certain radionuclides that have the same chemical properties as their non-radioactive isotopes and that can replace the latter in biologically-relevant molecules. After injection or inhalation of tiny amounts of these modified molecules, e.g., modified glucose (FDG) or neurotransmitters, their spatial distribution can be detected by a PET-scanner. This device is sensitive to radiation resulting from the annihilation of emitted positrons when they collide with ubiquitously-present electrons. Detected distribution information concerning metabolism or brain perfusion can be derived and visualized in tomograms. Spatial resolution is on the order of about 3-6 mm, and temporal resolution is on the order of several minutes to fractions of an hour.

Functional Near-Infrared Imaging

Another method for accepting user brain activity measurement data is functional near-infrared imaging (fNIR). fNIR is a spectroscopic neuro-imaging method for measuring the level of neuronal activity in the brain. The method is based on neuro-vascular coupling, i.e., the relationship between neuronal metabolic activity and oxygen level (oxygenated hemoglobin) in blood vessels in proximity to the neurons.

Time-resolved frequency-domain spectroscopy (the frequency-domain signal is the Fourier transform of the original, time-domain signal) may be used in fNIR to provide quantitation of optical characteristics of the tissue and therefore offer robust information about oxygenation. Diffuse optical tomography (DOT) in fNIR enables researchers to produce images of absorption by dividing the region of interest into thousands of volume units, called voxels, calculating the amount of absorption in each (the forward model) and then putting the voxels back together (the inverse problem). fNIR systems commonly have multiple sources and detectors, signifying broad coverage of areas of interest, and high sensitivity and specificity. fNIR systems today often consist of little more than a probe with fiber optic sources and detectors, a piece of dedicated hardware no larger than a small suitcase and a laptop computer. Thus, fNIR systems can be portable; indeed battery operated, wireless continuous wave fNIR devices have been developed at the Optical Brain Imaging Lab of Drexel University. fNIR employs no ionizing radiation and allows for a wide range of movement; it's possible, for example, for a subject to walk around a room while wearing a fNIR probe. fNIR studies have examined cerebral responses to visual, auditory and somatosensory stimuli, as well as the motor system and language, and subsequently begun to construct maps of functional activation showing the areas of the brain associated with particular stimuli and activities.

For example, a fNIR spectroscopy device (fNIRS) has been developed that looks like a headband and uses laser diodes to send near-infrared light through the forehead at a relatively shallow depth e.g., (two to three centimeters) to interact with the brain's frontal lobe. Light ordinarily passes through the body's tissues, except when it encounters oxygenated or deoxygenated hemoglobin in the blood. Light waves are absorbed by the active, blood-filled areas of the brain and any remaining light is diffusely reflected to fNIRS detectors. See “Technology could enable computers to ‘read the minds’ of users,” Physorg.com http://www.physorg.com/news110463755.html (1 Oct. 2007).

There are three types of fNIR: (1) CW—continuous wave—In this method, infrared light shines at the same intensity level during the measurement period. The detected signal is lower intensity static signal (dc valued); (2) FD—frequency domain—In this method, input signal is a modulated sinusoid at some frequency and detected output signal has changes in amplitude and phase; (3) TR—time resolved—In time resolve spectroscopy, a very short pulse is introduced to be measured and the pulse length is usually on the order of picoseconds. The detected signal is usually a longer signal and has a decay time.

In one approach, an infrared imager captures an image of a portion of the user. For example, the imager may capture a portion of the user's forehead. Infrared imaging may provide an indication of blood oxygen levels which in turn may be indicative of brain activity. With such imaging, the infrared imager may produce a signal indicative of brain activity. According to one method, hemoglobin oxygen saturation and relative hemoglobin concentration in a tissue may be ascertained from diffuse reflectance spectra in the visible wavelength range. This method notes that while oxygenated and deoxygenated hemoglobin contributions to light attenuation are strongly variable functions of wavelength, all other contributions to the attenuation including scattering are smooth wavelength functions and can be approximated by Taylor series expansion. Based on this assumption, a simple, robust algorithm suitable for real time monitoring of the hemoglobin oxygen saturation in the tissue was derived. This algorithm can be used with different fiber probe configurations for delivering and collecting light passed through tissue. See Stratonnikov et al., “Evaluation of blood oxygen saturation in vivo from diffuse reflectance spectra,” J. Biomed. Optics, vol. 6, pp. 457-467 (2001).

Functional Magnetic Resonance Imaging

Another method of accepting user brain activity measurement data may include measuring blood oxygen level dependent effects by, for example, functional magnetic resonance imaging (fMRI). fMRI involves the use of magnetic resonance scanners to produce sets of cross sections—tomograms—of the brain, detecting weak but measurable resonance signals that are emitted by tissue water subjected to a very strong magnetic field after excitation with a high frequency electromagnetic pulse. Acquired resonance signals can be attributed to their respective spatial origins, and cross sectional images can be calculated. The signal intensity, often coded as a gray value of a picture element, depends on water content and certain magnetic properties of the local tissue. In general, structural MR imaging is used to depict brain morphology with good contrast and high resolution. Visualizing brain function by MRI relies on the relationship between increased neural activity of a brain region and increased hemodynamic response or blood flow to that brain region. The increased perfusion of activated brain tissue is the basis of the so-called Blood Oxygenation Level Dependent (BOLD)-effect: hemoglobin, the oxygen carrying molecule in blood, has different magnetic properties depending on its oxygenation state. While oxyhemoglobin is diamagnetic, deoxyhemoglobin is paramagnetic, which means that it locally distorts the magnetic field, leading to a local signal loss. In activated brain tissue the increased oxygen consumption is accompanied by a blood flow response. Thus, during activation of a brain region, deoxyhemoglobin is partly replaced by oxyhemoglobin, leading to less distortion of the local magnetic field and increased signal intensity. Color-coded statistical parametric activation maps (SPMs) are typically generated from statistical analyses of fMRI time series comparing signal intensity during different activation states.

Temporal and spatial resolution of fMRI depends on both scanning technology and the underlying physiology of the detected signal intensity changes. Structural images are usually obtained with a resolution of at least lmmx lmmx 1 mm voxels (the equivalent of a pixel in a volume), while fMRI voxels typically have edge lengths of about 3-5 mm. Temporal resolution of fMRI is on the order of between 1 and 3 seconds. The cerebral blood flow (CBF) response to a brain activation is delayed by about 3-6 seconds. There is a balance between temporal and spatial resolution, allowing whole brain scans in less than 3 seconds, and non-invasiveness, permitting repeated measurements without adverse events. In addition, the choice of scanning parameters allows increasing one parameter at the expense of the other. Recent fMRI approaches show that for some neural systems the temporal resolution can be improved down to milliseconds and spatial resolution can be increased to the level of cortical columns as basic functional units of the cortex.

In one embodiment, an fMRI protocol may include fMRI data may be acquired with an MRI scanner such as a 3 T Magnetom Trio Siemens scanner. T2*-weighted functional MR images may be obtained using axially oriented echo-planar imaging. For each subject, data may be acquired in three scanning sessions or functional runs. The first four volumes of each session may be discarded to allow for T1 equilibration effects. For anatomical reference, a high-resolution T1-weighted anatomical image may be obtained. Foam cushioning may be placed tightly around the side of the subject's head to minimize artifacts from head motion. Data preprocessing and statistical analysis may be carried out using a statistical parametric mapping function, such as SPM99 (Statistical Parametric Mapping, Wellcome Institute of Cognitive Neurology, London, UK). Individual functional images may be realigned, slice-time corrected, normalized into a standard anatomical space (resulting in isotropic 3 mm voxels) and smoothed with a Gaussian kernel of 6 mm. In one embodiment, a standard anatomical space may be based on the ICBM 152 brain template (MNI, Montreal Neurological Institute). A block-design model with a boxcar regressor convoluted with the hemodynamic response function may be used as the predictor to compare activity related to a stimulus versus a control object. High frequency noise may be removed using a low pass filter (e.g., Gaussian kernel with 4.0 s FWHM) and low frequency drifts may be removed via a high pass filter. Effects of the conditions for each subject may be compared using linear contrast, resulting in a t-statistic for each voxel. A group analysis may be carried out on a second level using a whole brain random-effect analysis (one-sample t-test). Regions that contain a minimum of five contiguous voxels thresholded at P<0.001 (uncorrected for multiple comparisons) may be considered to be active. See Schaefer et al., “Neural correlates of culturally familiar brands of car manufacturers,” Neurolmage vol. 31, pp. 861-865 (2006).

Mapping Brain Activity

When brain activity data are collected from groups of individuals, data analysis across individuals may take into account variation in brain anatomy between and among individuals. To compare brain activations between individuals, the brains are usually spatially normalized to a template or control brain. In one approach they are transformed so that they are similar in overall size and spatial orientation. Generally, the goal of this transformation is to bring homologous brain areas into the closest possible alignment. In this context the Talairach stereotactic coordinate system is often used. The Talairach system involves a coordinate system to identify a particular brain location relative to anatomical landmarks; a spatial transformation to match one brain to another; and an atlas describing a standard brain, with anatomical and cytoarchitectonic labels. The coordinate system is based on the identification of the line connecting the anterior commissure (AC) and posterior commissure (PC)—two relatively invariant fiber bundles connecting the two hemispheres of the brain. The AC-PC line defines the y-axis of the brain coordinate system. The origin is set at the AC. The z-axis is orthogonal to the AC-PC-line in the foot-head direction and passes through the interhemispheric fissure. The x-axis is orthogonal to both the other axes and points from AC to the right. Any point in the brain can be identified relative to these axes.

Accordingly, anatomical regions may be identified using the Talairach coordinate system or the Talairach daemon (TD) and the nomenclature of Brodmann. The Talairach daemon is a high-speed database server for querying and retrieving data about human brain structure over the internet. The core components of this server are a unique memory-resident application and memory-resident databases. The memory-resident design of the TD server provides high-speed access to its data. This is supported by using TCP/IP sockets for communications and by minimizing the amount of data transferred during transactions. A TD server data may be searched using x-y-z coordinates resolved to 1×1×1 mm volume elements within a standardized stereotaxic space. An array, indexed by x-y-z coordinates, that spans 170 mm (x), 210 mm (y) and 200 mm (z), provides high-speed access to data. Array dimensions are approximately 25% larger than those of the Co-planar Stereotaxic Atlas of the Human Brain (Talairach and Tournoux, 1988). Coordinates tracked by a TD server are spatially consistent with the Talairach Atlas. Each array location stores a pointer to a relation record that holds data describing what is present at the corresponding coordinate. Data in relation records are either Structure Probability Maps (SP Maps) or Talairach Atlas Labels, though others can be easily added. The relation records are implemented as linked lists to names and values for brain structures. The TD server may be any computing device, such as a Sun Sparcstation 20 with 200 Mbytes of memory. Such a system provides 24-hour access to the data using a variety of client applications.

Some commercially available analysis software such as SPM5 (available for download from http://www.fil.ion.ucl.ac.uk/spm/software/spm5/) uses brain templates created by the Montreal Neurological Institute (MNI), based on the average of many normal MR brain scans. Although similar, the Talairach and the MNI templates are not identical, and care should be given to assigning localizations given in MNI coordinates correctly to, for example, cytoarchitectonically defined brain areas like the Brodmann areas (BA's), which are regions in the brain cortex defined in many different species based on its cytoarchitecture. Cytoarchitecture is the organization of the cortex as observed when a tissue is stained for nerve cells. Brodmann areas were originally referred to by numbers from 1 to 52. Some of the original areas have been subdivided further and referred to, e.g., as “23a” and “23b.” The Brodmann areas for the human brain include the following:

Areas 1, 2 & 3—Primary Somatosensory Cortex (frequently referred to as Areas 3, 1, 2 by convention)

Area 4—Primary Motor Cortex

Area 5—Somatosensory Association Cortex

Area 6—Pre-Motor and Supplementary Motor Cortex (Secondary Motor Cortex)

Area 7—Somatosensory Association Cortex

Area 8—Includes Frontal eye fields

Area 9—Dorsolateral prefrontal cortex

Area 10—Frontopolar area (most rostral part of superior and middle frontal gyri)

Area 11—Orbitofrontal area (orbital and rectus gyri, plus part of the rostral part of the superior frontal gyms)

Area 12—Orbitofrontal area (used to be part of BA11, refers to the area between the superior frontal gyms and the inferior rostral sulcus)

Area 13—Insular cortex

Area 17—Primary Visual Cortex (V1)

Area 18—Visual Association Cortex (V2)

Area 19—V3

Area 20—Inferior Temporal gyms

Area 21—Middle Temporal gyms

Area 22—Superior Temporal Gyms, of which the rostral part participates to Wernicke's area

Area 23—Ventral Posterior cingulate cortex

Area 24—Ventral Anterior cingulate cortex

Area 25—Subgenual cortex

Area 26—Ectosplenial area

Area 28—Posterior Entorhinal Cortex

Area 29—Retrosplenial cingular cortex

Area 30—Part of cingular cortex

Area 31—Dorsal Posterior cingular cortex

Area 32—Dorsal anterior cingulate cortex

Area 34—Anterior Entorhinal Cortex (on the Parahippocampal gyrus)

Area 35—Perirhinal cortex (on the Parahippocampal gyms)

Area 36—Parahippocampal cortex (on the Parahippocampal gyms)

Area 37—Fusiform gyms

Area 38—Temporopolar area (most rostral part of the superior and middle

temporal gyri

Area 39—Angular gyms, part of Wernicke's area

Area 40—Supramarginal gyms part of Wernicke's area

Areas 41 & 42—Primary and Auditory Association Cortex

Area 43—Subcentral area (between insula and post/precentral gyms)

Area 44—pars opercularis, part of Broca's area

Area 45—pars triangularis Broca's area

Area 46—Dorsolateral prefrontal cortex

Area 47—Inferior prefrontal gyms

Area 48—Retrosubicular area (a small part of the medial surface of the temporal lobe)

Area 52—Parainsular area (at the junction of the temporal lobe and the insula)

Associating Brain Activity with Brain Function or Mental State

The brain performs a multitude of functions. It is the location of memory, including working memory, semantic memory, and episodic memory. Attention is controlled by the brain, as is language, cognitive abilities, and visual-spatial functions. The brain also receives sensory signals and generates motor impulses. The frontal lobes of the brain are involved in most higher-level cognitive tasks as well as episodic and semantic memory. There is some degree of lateralization of the frontal lobes, e.g., the right frontal lobe is a locus for sustained attention and episodic memory retrieval, and the left frontal lobe is a locus for language, semantic memory retrieval, and episodic memory encoding.

The cingulated regions of the brain are associated with memory, initiation and inhibition of behavior, and emotion. The parietal regions of the brain are associated with attention, spatial perception and imagery, thinking involving time and numbers, working memory, skill learning, and successful episodic memory retrieval. The lateral temporal lobe of the brain is associated with language and semantic memory encoding and retrieval, while the medial temporal lobe is associated with episodic memory encoding and retrieval. The occipital temporal regions of the brain are associated with vision and visual-spatial processing.

Attention

Attention can be divided into five categories: sustained attention, selective attention, Stimulus-Response compatibility, orientation of attention, and division of attention. The tasks included in the sustained attention section involved continuous monitoring of different kinds of stimuli (e.g., somatosensory stimulation). The selective attention section includes studies in which subjects selectively attended to different attributes of the same set of stimuli (e.g., attend to color only for stimuli varying with respect to both color and shape). The stimulus-response (SR) compatibility section also includes studies examining selective attention, with the important difference that they involve a “conflict component.” In all cases, this is implemented by employing the Stroop task.

Prefrontal and parietal areas, preferentially in the right hemisphere, are frequently engaged during tasks requiring attention. An fMRI study involving a visual vigilance task was in close agreement with the results of a PET study showing predominantly right-sided prefrontal and parietal activation. Observed data is consistent with a right fronto-parietal network for sustained attention. Selective attention to one sensory modality is correlated with suppressed activity in regions associated with other modalities. For example, studies have found deactivations in the auditory cortex during attention area activations. Taken together, the results suggest the existence of a fronto-parietal network underlying sustained attention. Direct support for fronto-parietal interactions during sustained attention has been provided by structural equation modeling of fMRI data. Studies on the effects of attention on thalamic (intralaminar nuclei) and brain stem (midbrain tegmentum) activity have shown that these areas may control the transition from relaxed wakefulness to high general attention.

Selective attention is characterized by increased activity in posterior regions involved in stimulus processing. Different regions seem to be involved depending on the specific attribute that is attended to. Studies have shown attentional modulation of auditory regions, and modulation of activity in the lingual and fusiform gyri during a color attention task has also been demonstrated. Attending to motion activates a region in occipito-temporal cortex, and it has also been shown that, in addition to extrastriate regions, attention to motion increased activity in several higher-order areas as well. It may be that activity in extrastriate regions may be modulated by prefrontal, parietal and thalamic regions. Similarly, modulation of activity in specific posterior regions is mediated by regions in parietal and anterior cingulate cortices, as well as the pulvinar. A role of parietal cortex, especially the inferior parietal lobe, in control of selective attention has also been suggested. The prefrontal cortex may also play a role in attentional modulation. As long as attentional load is low, task-irrelevant stimuli are perceived and elicit neural activity, however, when the attentional load is increased, irrelevant perception and its associated activity is strongly reduced.

The stimulus-response compatibility panel includes selective attention studies on the Stroop test. The Stroop test is associated with activations in the anterior cingulate cortex. SR compatibility studies point to a role of both the anterior cingulate and the left prefrontal cortex. See Cabeza et al, “Imaging Cognition II: An Empirical Review of 275 PET and fMRI Studies,” J. Cognitive Neurosci., vol. 12, pp. 1-47 (2000).

Activation of the thalamic reticular nucleus is also associated with selective attention. See Contreras et al., “Inactivation of the Interoceptive Insula Disrupts Drug Craving and Malaise Induced by Lithium,” Science, vol. 318, pp. 655-658 (26 Oct. 2007).

The category “orientation of attention” includes studies associating shifts of spatial attention to parietal and prefrontal regions. Another study found activations in superior parietal regions during a visual search for conjunction of features. Based on the similarities in activation patterns, it appears that serial shifts of attention took place during the search task. There is also evidence for a large-scale neural system for visuospatial attention that includes the right posterior parietal cortex. PET and fMRI have been employed to study attentional orienting to spatial locations (left vs. right) and to time intervals (short vs. long stimulus onset times). Both spatial and temporal orienting were found to activate a number of brain regions, including prefrontal and parietal brain regions. Other analyses revealed that activations in the intraparietal sulcus were right-lateralized for spatial attention and left lateralized for temporal attention. Moreover, simultaneous spatial and temporal attention activate mainly parietal regions, suggesting that the parietal cortex, especially in the right hemisphere, is a site for interactions between different attentional processes. Parietal activation has also been demonstrated in an fMRI study of nonspatial attention shifting. In addition, the cerebellum has been implicated in attention shifting, and this is consistent with other findings of attentional activation of the cerebellum. It has also been shown that spatial direction of attention can influence the response of the extrastriate cortex. Specifically, it was demonstrated that while multiple stimuli in the visual field interact with each other in a suppressive way, spatially directed attention partially cancels out the suppressive effects.

With respect to division of attention, activity in the left prefrontal cortex increases under divided-attention conditions. In this context, it is also relevant to mention that if two tasks activate overlapping brain areas, there may be significant interference effects when the tasks are performed simultaneously. See Cabeza et al, “Imaging Cognition II: An Empirical Review of 275 PET and fMRI Studies,” J. Cognitive Neurosci., vol. 12, pp. 1-47 (2000).

Perception

Perception processes can be divided into object, face, space/motion, smell and “other” categories. Object perception is associated with activations in the ventral pathway (ventral brain areas 18, 19, and 37). The ventral occipito-temporal pathway is associated with object information, whereas the dorsal occipito-parietal pathway is associated with spatial information. For example, it has been shown that viewing novel, as well as familiar, line drawings, relative to scrambled drawings, activated a bilateral extrastriate area near the border between the occipital and temporal lobes. Based on these findings, it appears that this area is concerned with bottom-up construction of shape descriptions from simple visual features. It has also been shown that a region termed the “lateral occipital complex” (LO) is selectively activated by different kinds of shapes (e.g., shapes defined by motion, texture, and luminance contours). Greater activity in lingual gyms (Area 19) and/or inferior fusiform gyms (Area 37) is seen when subjects make judgments about appearance than when they make judgments about locations, providing confirmation that object identity preferentially activates regions in the ventral pathway. Both ventral and dorsal activations during shape-based object recognition suggests that visual object processing involves both pathways to some extent (a similar conclusion has been drawn based on network analysis of PET data).

Face perception involves the same ventral pathway as object perception, but there is a tendency for right-lateralization of activations for faces, but not for objects. For example, bilateral fusiform gyms activation is seen for faces, but with more extensive activation in the right hemisphere. Faces are perceived, at least in part, by a separate processing stream within the ventral object pathway. In an fMRI study, a region was identified that is more responsive to faces than to objects, termed the “fusiform face area” or FF area.

Whereas perception of objects and faces tends to preferentially activate regions in the ventral visual pathway, perception of spatial location tends to selectively activate more dorsal regions located in parietal cortex. Greater activity in the superior parietal lobe (area 7) as well as in the premotor cortex is seen during location judgments than during object judgments. The dorsal pathway is not only associated with space perception, but also with action. For example, perception of scripts of goal-directed hand action engage parts of the parietal cortex. Comparison have been done of meaningful actions (e.g., pantomime of opening a bottle) and meaningless actions (e.g., signs from the American Sign Language that were unknown to subjects). Whereas meaningless actions activated the dorsal pathway, meaningful actions activated the ventral pathway. Meaningless actions appear to be decoded in terms of spatiotemporal layout, while meaningful actions are processed by areas that allow semantic processing and memory storage. Thus, as object perception, location/action perception may involve both dorsal and ventral pathways to some extent.

Activations in the orbitofrontal cortex (where the secondary olfactory cortex is located), particularly in the right hemisphere, and the cerebellum are associated with smelling, as well as increased activity in the primary olfactory cortex (piriform cortex). Odorants (regardless of sniffing) activate the posterior lateral cerebellum, whereas sniffing (nonodorized air) activate anterior parts of the cerebellum. Thus the cerebellum receives olfactory information for modulating sniffing. Odorants (regardless of sniffing) activate the anterior and lateral orbitofrontal cortex whereas sniffing (even in the absence of odorants) activates the piriform and medial/posterior orbitofrontal cortices. In sum, smell perception involves primarily the orbitofrontal cortex and parts of the cerebellum and its neural correlates can be dissociated from those of sniffing.

With respect to the “other” category, fMRI has been employed to define a “parahippocampal place area” (PPA) that responds selectively to passively viewed scenes. A region probably overlapping with PPA responds selectively to buildings, and this brain region may respond to stimuli that have orienting value (e.g., isolated landmarks as well as scenes). The neural correlates of music perception have been localized to specialized neural systems in the right superior temporal cortex, which participate in perceptual analysis of melodies. Attention to changes in rhythm activate Broca's/insular regions in the left hemisphere, pointing to a role of this area in the sequencing of auditory input. Further, studies of “emotional perception” suggest that perception of different kinds of emotion are based on separate neural systems, with a possible convergence in prefrontal regions (area 47). Consistent with the role of the amygdala in fear conditioning, the amygdala is more activated for fearful faces relative to happy faces. See Cabeza et al, “Imaging Cognition II: An Empirical Review of 275 PET and fMRI Studies,” J. Cognitive Neurosci., vol. 12, pp. 1-47 (2000).

Imagery

Imagery can be defined as manipulating sensory information that comes not from the senses, but from memory. The memory representations manipulated can be in working memory (e.g., holding three spatial locations for 3 seconds), episodic memory (e.g., retrieving the location of an object in the study phase), or semantic memory (e.g., retrieving the shape of a bicycle). Thus, imagery-related contrasts could be classified within working memory, episodic retrieval, and semantic retrieval sections. Imagery contrasts can be described as visuospatial retrieval contrasts, and vice versa.

A central issue in the field of imagery has been whether those visual areas that are involved when an object is perceived are also involved when an object is imagined. In its strictest form, this idea would imply activation of the primary visual cortex in the absence of any visual input. A series of PET experiments provides support for similarities between visual perception and visual imagery by showing increased blood flow in Area 17 during imagery. In particular, by comparing tasks involving image formation for small and large letters, respectively, these studies provide evidence that imagery activates the topographically mapped primary visual cortex. A subsequent PET study, involving objects of three different sizes, provides additional support that visual imagery activates the primary visual cortex.

Increased activation in extrastriate visual regions is also associated with imaging tasks. The left inferior temporal lobe (area 37) is most reliably activated across subjects (for some subjects the activation extended into area 19 of the occipital lobe). Compared with a resting state, a left posterior-inferior temporal region was also activated. Moreover, mental imagery of spoken, concrete words has been shown to activate the inferior-temporal gyrus/fusiform gyms bilaterally. Thus, right temporal activation may be related to more complex visual imagery.

Color imagery and color perception engage overlapping networks anterior to region V4 (an area specialized for color perception), whereas areas V1-V4 were selectively activated by color perception. There is an increase in primary visual-cortex activity during negative imagery, as compared to neutral imagery. The primary visual cortex therefore appears to have a role in visual imagery, and emotion appears to affect the quality of the image representations.

Mental rotation of visual stimuli involves lateral parietal areas (BA47 and BA40). The bulk of the computation for this kind of mental rotation is performed in the superior parietal lobe. PET has been employed to study a mental-rotation task in which subjects were asked to decide whether letters and digits, tilted in 120°, 180°, or 240°, were in normal or mirror image form. The left parietal cortex is activated in this task.

Mental “exploration” of maps or routes has been studied using PET, revealing that this task is associated with increased activity in the right superior occipital cortex, the supplementary motor area (SMA) and the cerebellar vermis. The latter two activations are related to eye movements, and it appears that the superior occipital cortex has a specific role in generation and maintenance of visual mental images. In a subsequent PET study, occipital activation was again observed, although this time the peak was in left middle occipital gyms. This activation was specific to a task involving mental navigation—static visual imagery was not associated with occipital activation. Mental navigation tasks appears to tap visual memory to a high extent, and feedback influences from areas involved in visual memory may activate visual (occipital) areas during certain imagery tasks.

Thus, visual mental imagery is a function of the visual association cortex, although different association areas seem to be involved depending on the task demands. In addition, prefrontal areas have been activated in many of the reported comparisons. Partly, these effects may be driven by eye movements (especially for areas 6 and 8), but other factors, such as image generation and combination of parts into a whole, may account for some activations as well.

Neuroanatomical correlates of motor imagery via a mental writing task implicate a left parietal region in motor imagery, and, more generally, show similarities between mental writing and actual writing. Similarities between perception and imagery are seen in both musical imagery and perception. For example, relative to a visual baseline condition, an imagery task is associated with increased activity in the bilateral secondary auditory cortex. This was so despite the fact that the contrast included two entirely silent conditions. Similarly, a comparison of a task involving imaging a sentence being spoken in another person's voice with a visual control task reveals left temporal activation. Activation of the supplementary motor area was also seen, suggesting that both input and output speech mechanisms are engaged in auditory mental imagery. See Cabeza et al, “Imaging Cognition II: An Empirical Review of 275 PET and fMRI Studies,” J. Cognitive Neurosci., vol. 12, pp. 1-47 (2000).

Language

Language mapping studies are commonly divided into four categories: spoken and written word recognition crossed with spoken or no-spoken response. Word recognition, regardless of input modality and whether or not a spoken response is required, has consistently been found to activate areas 21 and 22 in the temporal cortex. In general, this activation tends to be bilateral, although in the category of written word recognition all activations are left-lateralized. The cortical surface covered by these areas is most likely made up by several distinct regions that can be functionally dissociated. Involvement of left superior temporal gyrus/Wernicke's area in word recognition is in agreement with the traditional view implicating this area in comprehension.

Whereas left temporal brain regions have been associated with word comprehension, left inferior prefrontal cortex/Broca's area has traditionally been linked to word production. However, comparing conditions involving spoken response with conditions involving no spoken response do not suggest that (left) prefrontal involvement is greater when spoken responses are required. Instead, the major difference between these two classes is that conditions involving spoken responses tend to activate the cerebellum to a higher extent. Broca's area is involved in word perception, as well as in word production, and in addition to having an output function, the left prefrontal areas may participate in receptive language processing in the uninjured state. An fMRI study has shown that cerebellar activation is related to the articulatory level of speech production.

Visual areas are more frequently involved in the case of written word recognition, and regardless of output (spoken/no spoken), written word recognition tends to differentially activate left prefrontal and anterior cingulate regions. Moreover, left inferior prefrontal activation has been associated with semantic processing.

A posterior left temporal region (BA 37) is a multimodal language region. Both blind and sighted subjects activate this area during tactile vs. visual reading (compared to non-word letter strings). This area may not contain linguistic codes per se, but may promote activity in other areas that jointly lead to lexical or conceptual access. Area 37 has been activated in several studies of written word recognition but not in studies of spoken word recognition. Lip-reading activates the auditory cortex in the absence of auditory speech sounds. The activation was observed for silent speech as well as pseudo-speech, but not for nonlinguistic facial movements, suggesting that lip-reading modulates the perception of auditory speech at a prelexical level.

There are few differences between sign language and spoken language, and sign language in bilingual persons activates a similar network as that underlying spoken language. The difference in activation in ventral temporal cortex (area 37) related to sign language appears to relate to an attention mechanism that assigns importance to signing hands and facial expressions. With respect to the processing of native and foreign languages, native-language processing, relative to processing of a foreign language, selectively activates several brain regions leading to the conclusion that some brain areas are shaped by early exposure to the maternal language, and that these regions may not be activated when people process a language that they have learned later in life. In Broca's area, second languages acquired in adulthood are spatially separated from native languages, whereas second languages acquired at an early age tend to activate overlapping regions within Broca's area. In Wernicke's area, no separation based on age of language acquisition is observed. Further, fMRI has been used to determine brain activity related to aspects of language processing. During phonological tasks, brain activation in males was lateralized to the left inferior frontal gyms, whereas the pattern was more diffuse for females.

Activation patterns related to the processing of particular aspects of information show that a set of brain regions in the right hemisphere is selectively activated when subjects try to appreciate the moral of a story as opposed to semantic aspects of the story. Brain activation associated with syntactic complexity of sentences indicates that parts of Broca's area increase their activity when sentences increase in syntactic complexity. See Cabeza et al, “Imaging Cognition II: An Empirical Review of 275 PET and fMRI Studies,” J. Cognitive Neurosci., vol. 12, pp. 1-47 (2000).

Working Memory

Working memory consists of three main components: a phonological loop for the maintenance of verbal information, a visuospatial sketchpad for the maintenance of visuospatial information, and a central executive for attentional control. Dozens of functional neuroimaging studies of working memory have been carried out. Working memory is associated with activations in prefrontal, parietal, and cingulate regions. There also may be involvement of occipital and cerebellar regions discriminations between different Brodmann's areas.

Working memory is almost always associated with increased activity in the prefrontal cortex. This activity is typically found in areas 6, 44, 9 and 46. Area 44 activations are more prevalent for verbal/numeric tasks than for visuospatial tasks, and tend to be lateralized to the left hemisphere (i.e., Broca's area), suggesting that they reflect phonological processing. Area 6 activations are common for verbal, spatial, and problem-solving tasks, and, hence, they are likely related to general working memory operations (i.e., they are not material or task-specific). In contrast, activations in areas 9 and 46 seem to occur for certain kinds of working memory tasks but not others. Activations in these two areas tend to be more prevalent for tasks that require manipulation of working memory contents, such as N-back tasks, than for tasks that require only uninterrupted maintenance, such as delayed response tasks. Ventrolateral prefrontal regions are involved in simple short-term operations, whereas mid-dorsal prefrontal regions perform higher-level executive operations, such as monitoring. Object working memory may be left-lateralized while spatial-working memory is right-lateralized.

In addition to prefrontal activations, working memory studies normally show activations in parietal regions, particularly areas 7 and 40. In the case of verbal/numeric tasks, these activations tend to be left-lateralized, suggesting that they are related to linguistic operations. The phonological loop consists of a phonological store, where information is briefly stored, and a rehearsal process, which refreshes the contents of this store. Left parietal activations may reflect the phonological store, whereas left prefrontal activations in area 44 (Broca's area) may reflect the rehearsal process. When nonverbal materials are employed, parietal activations, particularly those in area 7, tend to be bilateral, and to occur for spatial but not for object working memory. Thus the distinction between a ventral pathway for object processing and a dorsal pathway for spatial processing may also apply to working memory.

Working memory tasks are also associated with anterior cingulate, occipital, and cerebellar activations. Anterior cingulate activations are often found in Area 32, but they may not reflect working memory operations per se. Activity in dorsolateral prefrontal regions (areas 9 and 46) varies as a function of delay, but not of readability of a cue, and activity in the anterior cingulate (and in some right ventrolateral prefrontal regions) varies as a function of readability but not of delay of a cue. Thus, the anterior cingulate activation seems to be related to task difficulty, rather than to working memory per se. Occipital activations are usually found for visuospatial tasks, and may reflect increased visual attention under working memory conditions. Cerebellar activations are common during verbal working memory tasks, particularly for tasks involving phonological processing (e.g., holding letters) and tasks that engage Broca's area (left area 44).

Consistent with the idea that mid-dorsal areas 9/46 are involved in higher-level working memory operations, activations in these areas are prominent in the reasoning and planning tasks. Area 10 activations are also quite prevalent, and may be related to episodic memory aspects of problem-solving tasks (see episodic memory retrieval section above). Tasks involving sequential decisions, such as conceptual reasoning and card sorting consistently engage the basal ganglia, thalamic, and cerebellar regions. These regions are typical skill learning regions and may reflect the skill-learning aspects of sequential problem-solving tasks. Also, the basal ganglia, thalamus, and prefrontal cortex are intimately linked and dysfunction of this circuitry could underlie planning deficits in Parkinson disease. See Cabeza et al, “Imaging Cognition II: An Empirical Review of 275 PET and fMRI Studies,” J. Cognitive Neurosci., vol. 12, pp. 1-47 (2000).

Semantic Memory Retrieval

Semantic memory refers to knowledge we share with other members of our culture, such as knowledge about the meaning of words (e.g., a banana is a fruit), the properties of objects (e.g., bananas are yellow), and facts (e.g., bananas grow in tropical climates). Semantic memory may be divided into two testing categories, categorization tasks and generation tasks. In categorization tasks, subjects classify words into different categories (e.g., living vs. nonliving), whereas in generation tasks, they produce one (e.g., word stem completion) or several (for example, fluency tasks) words in response to a cue. Semantic memory retrieval is associated with activations in prefrontal, temporal, anterior cingulate, and cerebellar regions.

Prefrontal activity during semantic memory tasks frequently found in the left hemisphere but not in the right. This is so even when the stimuli are nonverbal materials, such as objects and faces. This striking left-lateralization is in sharp contrast with the right-lateralization of prefrontal activity typically observed during episodic memory retrieval. This asymmetric pattern has been conceptualized in terms of a hemispheric encoding/retrieval asymmetry (HERA) model. This model consists of three hypotheses: (1) the left prefrontal cortex is differentially more involved in semantic memory retrieval than is the right prefrontal cortex; (2) the left prefrontal cortex is differentially more involved in encoding information into episodic memory than is the right prefrontal cortex; and (3) the right prefrontal cortex is differentially more involved in episodic memory retrieval than is the left prefrontal cortex. Thus, the left-lateralization of prefrontal activations supports the first hypothesis of the model. The second and third hypotheses are addressed by episodic memory encoding and episodic memory retrieval testing, respectively, as discussed above.

Within the frontal lobes, activations are found in most prefrontal regions, including ventrolateral (areas 45 and 47), ventromedial (area 11), posterior (areas 44 and 6), and mid-dorsal (areas 9 and 46) regions. Activations in ventrolateral regions occur during both classification and generation tasks and under a variety of conditions, suggesting that they are related to generic semantic retrieval operations. In contrast, area 11 activations are more common for classification than for generation tasks, and could be related to a component of classification tasks, such as decision-making. Conversely, activations in posterior and dorsal regions are more typical for generation tasks than for classification tasks. Many posterior activations (areas 44 and 6) occur at or near Broca's area, thus they may reflect overt or covert articulatory processes during word generation. Activations in dorsal regions (areas 9 and 46) are particularly frequent for fluency tasks. Because fluency tasks require the monitoring of several items in working memory, these activations may reflect working memory, rather than semantic memory, per se. Accordingly, when subjects complete word stems, areas 9/10 are more active for stems with many completions than for stems with few completions. These areas may therefore be involved in selecting among competing candidate responses.

Semantic retrieval tasks are also commonly associated with temporal, anterior cingulate, and cerebellar regions. Temporal activations occur mainly in the left middle temporal gyms (area 21) and in bilateral occipito-temporal regions (area 37). Left area 21 is activated not only for words but also pictures and faces, suggesting it is involved in higher-level semantic processes that are independent of input modality. In contrast, area 37 activations are more common for objects and faces, so they could be related to the retrieval of visual properties of these stimuli. Anterior cingulate activations are typical for generation tasks. The anterior cingulate—like the dorsal prefrontal cortex—is more active for stems with many than with few completions, whereas the cerebellum shows the opposite pattern. The anterior cingulate may therefore be involved in selecting among candidate responses, while the cerebellum may be involved in memory search processes. Accordingly cerebellar activations are found during single-word generation, but not during fluency tasks.

The retrieval of animal information is associated with left occipital regions and the retrieval of tool information with left prefrontal regions. Occipital activations could reflect the processing of the subtle differences in physical features that distinguish animals, whereas prefrontal activations could be related to linguistic or motor aspects of tool utilization. Animal knowledge activates a more anterior region (area 21) of the inferior temporal lobe than the one associated with tool knowledge (area 37). Whereas generating color words activates fusiform areas close to color perception regions, generating action words activates a left temporo-occipital area close to motion perception regions. Thus knowledge about object attributes is stored close to the regions involved in perceiving these attributes. See Cabeza et al, “Imaging Cognition II: An Empirical Review of 275 PET and fMRI Studies,” J. Cognitive Neurosci., vol. 12, pp. 1-47 (2000).

Episodic Memory Encoding

Episodic memory refers to memory for personally experienced past events, and it involves three successive stages: encoding, storage, and retrieval. Encoding refers to processes that lead to the formation of new memory traces. Storage designates the maintenance of memory traces over time, including consolidation operations that make memory traces more permanent. Retrieval refers to the process of accessing stored memory traces. Encoding and retrieval processes are amenable to functional neuroimaging research, because they occur at specific points in time, whereas storage/consolidation processes are not, because they are temporally distributed. It is very difficult to differentiate the neural correlates of encoding and retrieval on the basis of the lesion data, because impaired memory performance after brain damage may reflect encoding deficits, retrieval deficits, or both. In contrast, functional neuroimaging allows separate measures of brain activity during encoding and retrieval.

Episodic encoding can be intentional, when subjects are informed about a subsequent memory test, or incidental, when they are not. Incidental learning occurs, for example, when subjects learn information while performing a semantic retrieval task, such as making living/nonliving decisions. Semantic memory retrieval and incidental episodic memory encoding are closely associated. Semantic processing of information (semantic retrieval) usually leads to successful storage of new information. Further, when subjects are instructed to learn information for a subsequent memory test (intentional encoding), they tend to elaborate the meaning of the information and make associations on the basis of their knowledge (semantic retrieval). Thus, most of the regions (for example, left prefrontal cortex) associated with semantic retrieval tasks are also associated with episodic memory encoding.

Episodic encoding is associated primarily with prefrontal, cerebellar, and medial temporal brain regions. In the case of verbal materials, prefrontal activations are always left lateralized. This pattern contrasts with the right lateralization of prefrontal activity during episodic retrieval for the same kind of materials. In contrast, encoding conditions involving nonverbal stimuli sometimes yield bilateral and right-lateralized activations during encoding. Right-lateralized encoding activations may reflect the use of non-nameable stimuli, such as unfamiliar faces and textures, but encoding of non-nameable stimuli has been also associated with left-lateralized activations with unfamiliar faces and locations. Contrasting encoding of verbal materials with encoding of nonverbal materials may speak to the neural correlates of different materials rather than to the neural correlates of encoding per se.

The prefrontal areas most commonly activated for verbal materials are areas 44, 45, and 9/46. Encoding activations in left area 45 reflects semantic processing while those in left area 44 reflects rote rehearsal. Areas 9/46 may reflect higher-order working memory processes during encoding. Activation in left area 9 increases as a function of organizational processes during encoding, and is attenuated by distraction during highly organizational tasks. Cerebellar activations occur only for verbal materials and show a tendency for right lateralization. The left-prefrontal/right-cerebellum pattern during language, verbal-semantic memory, and verbal-episodic encoding tasks is consistent with the fact that fronto-cerebellar connections are crossed.

Medial-temporal activations are seen with episodic memory encoding and can predict not only what items will be remembered, but also how well they will be remembered. Medial-temporal activations show a clear lateralization pattern: they are left-lateralized for verbal materials and bilateral for nonverbal materials. Under similar conditions, medial-temporal activity is stronger during the encoding of pictures than during the encoding of words, perhaps explaining why pictures are often remembered better than words. In the case of nonverbal materials, medial-temporal activity seems to be more pronounced for spatial than for nonspatial information, consistent with the link between the hippocampus and spatial mapping shown by animal research. See Cabeza et al, “Imaging Cognition II: An Empirical Review of 275 PET and fMRI Studies,” J. Cognitive Neurosci., vol. 12, pp. 1-47 (2000).

Episodic Memory Retrieval

Episodic memory retrieval refers to the search, access, and monitoring of stored information about personally experienced past events, as well as to the sustained mental set underlying these processes. Episodic memory retrieval is associated with seven main regions: prefrontal, medial temporal, medial parieto-occipital, lateral parietal, anterior cingulate, occipital, and cerebellar regions.

Prefrontal activations during episodic memory retrieval are sometimes bilateral, but they show a clear tendency for right-lateralization. The right lateralization of prefrontal activity during episodic memory retrieval contrasts with the left lateralization of prefrontal activity during semantic memory retrieval and episodic memory encoding. Left prefrontal activations during episodic retrieval tend to occur for tasks that require more reflectively complex processing. These activations may be related to semantic retrieval processes during episodic retrieval. Semantic retrieval can aid episodic retrieval particularly during recall, and bilateral activations tend to be more frequent during recall than during recognition. Moreover, left prefrontal activity during episodic retrieval is associated with retrieval effort, and is more common in older adults than in young adults.

Prefrontal activity changes as a function of the amount of information retrieved during the scan have been measured by varying encoding conditions (e.g., deep vs. shallow), or by altering the proportion of old items (e.g., targets) during the scan. As more information is retrieved during the scan, prefrontal activity may increase (retrieval success), decrease (retrieval effort), or remain constant (retrieval mode). These three outcomes are not necessarily contradictory; they may correspond to three different aspects of retrieval: maintaining an attentional focus on a particular past episode (retrieval mode), performing a demanding memory search (retrieval effort), and monitoring retrieved information (retrieval success).

These different aspects of retrieval may map to distinct prefrontal regions. The region most strongly associated to retrieval mode is the right anterior prefrontal cortex (area 10). A combined PET/ERP study associated a right area 10 activation with task-related rather than item-related activity during episodic retrieval. Activations associated with retrieval effort show a tendency to be left lateralized, specifically in left areas 47 and 10. Bilateral Areas 10, 9, and 46 are sometimes associated with retrieval success. Prefrontal activity is also seen to increase with success activations when subjects are warned about the proportion of old and new items during the scan (biasing).

Medial-temporal activations have been seen in the typical pattern of episodic retrieval in PET and fMRI studies, for both verbal and nonverbal materials. In contrast with medial-temporal activations during episodic encoding, those during episodic retrieval tend to occur in both hemispheres, regardless of the materials employed. That they are sometimes found in association with retrieval success, but never in association with retrieval effort or retrieval mode, suggest that they are related to the level of retrieval performance. Medial-temporal activity increases as linear function of correct old word recognition, and this activity may reflect successful access to stored-memory representations. Further, hippocampal activity has been associated with conscious recollection. Hippocampal activity is also sensitive to the match between study and test conditions, such as the orientation of study and test objects. However, recollection need not be accurate; for example in the case of significant hippocampal activations during the recognition of false targets. Accurate recognition yields additional activations in a left temporoparietal region, possibly reflecting the retrieval of sensory properties of auditorily studied words. Further, intentional retrieval is not a precondition for hippocampal activity; activations in this area are found for old information encountered during a non-episodic task, suggesting that they can also reflect spontaneous reminding of past events.

After the right prefrontal cortex, the most typical region in PET/fMRI studies of episodic retrieval is the medial parieto-occipital area that includes retrosplenial (primarily areas 29 and 30), precuneus (primarily medial area 7 and area 31), and cuneus (primarily medial areas 19, 18, and 17) regions. The critical role of the retrosplenial cortex in memory retrieval is supported by evidence that lesions in this region can cause severe memory deficits (e.g., retrosplenial amnesia. The role of the precuneus has been attributed to imagery and to retrieval success. Retrieval-related activations in the precuneus are more pronounced for imageable than for nonimageable words. However, the precuneus region was not more activated for object recall than for word recall. Imagery-related activations are more anterior than activations typically associated with episodic retrieval. The precuneus is activated for both imageable and abstract words, and for both visual and auditory study presentations. Thus this region appears to be involved in episodic retrieval irrespective of imagery content. The precuneus cortex is more active in a high-target than in low-target recognition condition.

Episodic memory retrieval is also associated with activations in lateral parietal, anterior cingulate, occipital, and cerebellar regions. Lateral parietal regions have been associated with the processing of spatial information during episodic memory retrieval and with the perceptual component of recognition. Anterior cingulate activations (areas 32 and 24) have been associated with response selection and initiation of action. Anterior cingulate activations may be related to language processes because they are more frequent for verbal than for nonverbal materials. As expected, occipital activations are more common during nonverbal retrieval, possibly reflecting not only more extensive processing of test stimuli but also memory-related imagery operations. Cerebellar activations have been associated with self-initiated retrieval operations. This idea of initiation is consistent with the association of cerebellar activations with retrieval mode and effort, rather than with retrieval success.

With respect to context memory, a fusiform region is more active for object identity than for location retrieval, whereas an inferior parietal region shows the opposite pattern. Thus the ventral/dorsal distinction applies also to episodic retrieval. In the time domain, recognition memory (what) has been contrasted with recency memory (when). Medial-temporal regions are more active during item memory than during temporal-order memory, whereas dorsal prefrontal and parietal regions are more active during temporal-order memory than during item memory. Parietal activations during temporal-order memory suggest that the dorsal pathway may be associated not only with “where” but also with “when.”

Prefrontal regions were similarly activated in both recall and recognition tests. This may signify the use of associative recognition—a form of recognition with a strong recollection component, or to the careful matching of task difficulty in the two tests. A comparison of free and cued recall found a dissociation in the right prefrontal cortex between dorsal cortex (areas 9 and 46), which is more active during free recall, and the ventrolateral cortex (area 47/frontal insula), which is more active during cued recall. Thus some of the activations observed during episodic-memory retrieval tasks may reflect the working-memory components of these tasks. Autobiographic retrieval is associated with activations along a right fronto-temporal network.

Episodic memory retrieval is associated with activations in prefrontal, medial temporal, posterior midline, parietal, anterior cingulate, occipital, and cerebellar regions. Prefrontal activations tend to be right-lateralized, and have been associated with retrieval mode, retrieval effort, and retrieval success. The engagement of medial temporal regions has been linked to retrieval success and recollection. Posterior midline activations also seem related to retrieval success. Parietal activations may reflect processing of spatial context, and anterior cingulate activations may reflect selection/initiation processes. Cerebellar involvement has been attributed to self-initiated retrieval. Spatial retrieval engaged parietal regions, and object retrieval activated temporal regions. Parietal regions are also activated during temporal-order retrieval, suggesting a general role in context memory. See Cabeza et al, “Imaging Cognition II: An Empirical Review of 275 PET and fMRI Studies,” J. Cognitive Neurosci., vol. 12, pp. 1-47 (2000).

Priming

Priming can be divided into perceptual and conceptual priming. In several studies, perceptual priming has been explored by studying completion of word-stems. In the primed condition, it is possible to complete the stems with previously presented words, whereas this is not possible in the unprimed condition. Visual perceptual priming is associated with decreased activity in the occipital cortex. PET and fMRI studies on non-verbal visual perceptual priming have revealed priming-related reduction in activation of regions in the occipital and inferior temporal brain regions. Priming effects can persist over days; repetition priming (item-specific learning) as measured by fMRI shows that learning-related neural changes that accompany these forms of learning partly involve the same regions.

Comparisons of blood flow responses associated with novel vs. familiar stimuli (across memory tasks) show that novel stimuli are associated with higher activity in several regions, including fusiform gyms and cuneus. Thus, priming-related reductions in activity in visual areas occur even after subliminal presentation.

Priming cannot only facilitate perceptual processes, but may also influence conceptual processes. The primed condition is associated with decreased activity in several regions, including the left inferior prefrontal cortex. Similarly, several fMRI studies that have included repeated semantic processing of the same items have found reduced left prefrontal activation associated with the primed condition. Left prefrontal reduction of activation is not seen when words are non-semantically reprocessed, suggesting that the effect reflects a process-specific change (not a consequence of mere repeated exposure). This process-specific effect can be obtained regardless of the perceptual format of the stimuli (e.g., pictures or words). Many memory tests rely upon a mixture of processes, and even the stem-completion task, which has been used in several studies of perceptual priming, has been associated with priming-related left prefrontal reductions. This may be taken as evidence that this task, too, taps both perceptual and conceptual processes.

With respect to a neural correlate of priming, repeating items during performance of the same task, or even during performance of different tasks, can lead to decreases in the amount of activation present in specific brain areas. This effect may reflect enhanced processing of the involved neurons or/and a specification of the involved neuronal population, resulting in a spatially less diffuse response. See Cabeza et al, “Imaging Cognition II: An Empirical Review of 275 PET and fMRI Studies,” J. Cognitive Neurosci., vol. 12, pp. 1-47 (2000).

Procedural Memory

Procedural memory processes can be divided into three subcategories: conditioning, motor-skill learning, and nonmotor skill learning. With respect to conditioning, studies on eye-blink conditioning point to a consistent role of the cerebellum in this form of learning (e.g., decreased activity in the cerebellum following conditioning). Conditioning is also associated with increased activity in the auditory cortex.

Motor-skill learning is associated with activation of motor regions. Area 6 is involved, and learning-related changes have also repeatedly been demonstrated in the primary motor cortex (area 4). The size of the activated area in the primary motor cortex increases as a function of training. There is also parietal involvement in motor skill learning; fronto-parietal interactions may underlie task performance. With respect to nonmotor skill learning, cerebellar activation is observed across tasks, as is consistent involvement of parietal brain regions. This is in line with the pattern observed for motor-skill learning, and the overlap in activation patterns may reflect common processes underlying these two forms of procedural memory. See Cabeza et al, “Imaging Cognition II: An Empirical Review of 275 PET and fMRI Studies,” J. Cognitive Neurosci., vol. 12, pp. 1-47 (2000).

Preference

Neural correlates of preference can be detected through neuroimaging studies. For example, in a simulated buying decision task between similar fast moving consumer goods, only a subject's preferred brand elicited a reduced activation in the dorsolateral prefrontal, posterior parietal and occipital cortices and the left premotor area (Brodmann areas 9, 46, 7/19 and 6), and only when the target brand was the subjects' favorite one. Simultaneously, activity was increased in the inferior precuneus and posterior cingulated (BA 7), right superior frontal gyms (BA 10), right supramarginal gyms (BA 40) and most pronounced in the ventromedial prefrontal cortex (“VMPFC”, BA 10).

In fMRI analyses, activation of the nucleus accumbens is associated with product preference, and the medial prefrontal cortex is associated with evaluation of gains and losses. When these areas of the brain are activated, subjects bought a product at an accuracy rate of 60%. In other fMRI analyses, early stage romantic love has been associated with activation of subcortical reward regions such as the right ventral tegmental area and the dorsal caudate area. Subjects in more extended romantic love showed more activity in the ventral pallidum. In still another fMRI analysis, in subjects experiencing a mistake, activation of the rostral anterior cingulated cortex increased in proportion to a financial penalty linked to the mistake. See Wise, “Thought Police: How Brain Scans Could Invade Your Private Life,” Popular Mechanics, (November 2007).

With respect to brand discrimination, brain activations in product choice differ from those for height discrimination, and there is a positive relationship between brand familiarity and choice time. Neural activation during choice tasks involves brain areas responsible for silent vocalization. Decision processes take approximately 1 second as measured by magnetoencephalography and can be seen as two halves. The first period involves gender-specific problem recognition processes, and the second half concerns the choice itself (no gender differences). MEG measurements can be categorized in four stages:

Stage 1—V (visual): Activation of the primary visual cortices at around 90 ms after stimulus onset.

Stage 2—T (temporal): Neuronal activity predominantly over left anterior-temporal and middle-temporal cortices at approximately 325 ms after stimulus onset. Some specific activity was also found over the left frontal and right extra-striate cortical areas.

Stage 3—F (frontal): Activation of the left inferior frontal cortices at about 510 ms after stimulus onset. These signals are consistent with activation of Broca's speech area.

Stage 4—P (parietal): Activation of the right posterior parietal cortices (P) at around 885 ms after stimulus onset.

Male brain activity differed from female in the second stage (T) but not in the other three stages (V, F and P). Left anterior temporal activity is present in both groups, but males seem to activate right hemispherical regions much more strongly during memory recall than females do. As noted above, response times also differed for male and female subjects. See Amber et al., “Salience and Choice: Neural Correlates of Shopping Decisions,” Psychology & Marketing, Vol. 21(4), pp. 247-261 (April 2004).

In an fMRI study, a consistent neural response in the ventromedial prefrontal cortex was associated with subjects' behavioral preferences for sampled anonymized beverages. In a brand-cued experiment, brand knowledge of one of the beverages had a dramatic influence on expressed behavioral preferences and on the measured brain responses. See Kenning et al., “Neuroeconomics: an overview from an economic perspective,” Brain Res. Bull., vol. 67, pp. 343-354 (2005).

In an fMRI study, only the presence of a subject's favorite brand indicating a distinctive mode of decision-making was associated with activation of regions responsible for integrating emotions. See Kenning et al., “Neuroeconomics: an overview from an economic perspective,” Brain Res. Bull., vol. 67, pp. 343-354 (2005).

Emotion

Various emotions may be identified through detection of brain activity. As discussed below, activation of the anterior insula has been associated with pain, distress, and other negative emotional states. Conversely, as discussed below, positive emotional processes are reliably associated with a series of structures representing a reward center, including the striatum and caudate, and areas of the midbrain and cortex to which they project, such as the ventromedial prefrontal cortex, orbitofrontal cortex, and anterior cingulated cortex, as well as other areas such as the amygdala and the insula.

In addition, approval and/or disapproval may be determined based on brain activity. For example, in an fMRI study, blood-oxygen-level-dependent signal changes were measured in subjects viewing facial displays of happiness, sadness, anger, fear, and disgust, as well as neutral faces. Subjects were tasked with discriminating emotional valence (positive versus negative) and age (over 30 versus under 30) of the faces. During the task, normal subjects showed activation in the fusiform gyms, the occipital lobe, and the inferior frontal cortex relative to the resting baseline condition. The increase was greater in the amygdala and hippocampus during the emotional valence discrimination task than during the age discrimination task. See Gur et al., “An fMRI study of Facial Emotion Processing in Patients with Schizophrenia,” Am. J. Psych., vol. 159, pp. 1992-1999 (2002).

Frustration is associated with decreased activation in the ventral striatum, and increased activation in the anterior insula and the right medial prefrontal cortex by fMRI. See Kenning et al., “Neuroeconomics: an overview from an economic perspective,” Brain Res. Bull., vol. 67, pp. 343-354 (2005).

Fairness, Altruism and Trust

fMRI has been used to show that perceived unfairness correlates with activations in the anterior insula and the dorsolateral, prefrontal cortex (“DLPFC”). Anterior insula activation is consistently seen in neuroimaging studies focusing on pain and distress, hunger and thirst, and autonomic arousal. Activation of the insula has also been associated with negative emotional states, and activation in the anterior insula has been linked to a negative emotional response to an unfair offer, indicating an important role for emotions in decision-making.

In contrast to the insula region, the DLPFC has been linked to cognitive processes such as goal maintenance and executive control. Thus, DLPFC activation may indicate objective recognition of benefit despite an emotional perception of unfairness.

Event-related hyperscan-fMRI (“hfMRI” which means that two volunteers are measured parallel in two scanners) has been used to measure the neural correlates of trust. By this method, the caudate nucleus has been shown to be involved in trust-building and reciprocity in economic exchange. The caudate nucleus is commonly active when learning about relations between stimuli and responses. See Kenning et al., “Neuroeconomics: an overview from an economic perspective,” Brain Res. Bull., vol. 67, pp. 343-354 (2005).

In a PET study, sanctions against defectors were associated with activity in reward-processing brain regions. See Kenning et al., “Neuroeconomics: an overview from an economic perspective,” Brain Res. Bull., vol. 67, pp. 343-354 (2005).

Reward

In an fMRI study, activation changes in the sublenticular extended amygdala (SLEA) and orbital gyms were associated with expected values of financial gain. Responses to actual experience of rewards increased monotonically with monetary value in the nucleus accumbens, SLEA, and thalamus. Responses to prospective rewards and outcomes were generally, but not always, seen in the same regions. Overlaps with activation changes seen previously in response to tactile stimuli, gustatory stimuli, and euphoria-inducing drugs were found. See Kenning et al., “Neuroeconomics: an overview from an economic perspective,” Brain Res. Bull., vol. 67, pp. 343-354 (2005).

In another fMRI study, within a group of cooperative subjects the prefrontal cortex showed activation changes when subjects playing a human compared to playing a computer. Within a group of non-cooperators, no significant activation changes in the prefrontal cortex were seen between computer and human conditions. See Kenning et al., “Neuroeconomics: an overview from an economic perspective,” Brain Res. Bull., vol. 67, pp. 343-354 (2005).

In an fMRI study, products symbolizing wealth and status were associated with increased activity in reward-related brain areas. See Kenning et al., “Neuroeconomics: an overview from an economic perspective,” Brain Res. Bull., vol. 67, pp. 343-354 (2005).

In a PET study, participants were risk averse in gains and risk-seeking in losses; and ambiguity-seeking in neither gains nor losses. Interactions between attitudes and beliefs were associated with neural activation changes in dorsomedial and ventromedial brain areas. See Kenning et al., “Neuroeconomics: an overview from an economic perspective,” Brain Res. Bull., vol. 67, pp. 343-354 (2005).

In an fMRI study, increasing monetary gains were associated with increased activity in a subcortical region of the ventral striatum in a magnitude-proportional manner. This ventral striatal activation was not evident during anticipation of losses. Actual gain outcomes were associated with activation of a region of the medial prefrontal cortex. During anticipation of gain, ventral striatal activation was associated with feelings characterized by increasing arousal and positive valence. See Kenning et al., “Neuroeconomics: an overview from an economic perspective,” Brain Res. Bull., vol. 67, pp. 343-354 (2005).

In an fMRI study, activation of parts of the limbic system were associated with decisions involving immediate rewards. Activity changes in the lateral prefrontal cortex and posterior parietal cortex were associated with inter-temporal choices. Greater relative fronto-parietal activity was associated with a subject's choice of longer term options. See Kenning et al., “Neuroeconomics: an overview from an economic perspective,” Brain Res. Bull., vol. 67, pp. 343-354 (2005).

Brain Activation By Region

Prefrontal Regions

The prefrontal cortex is involved in almost all high-level cognitive tasks. Prefrontal activations are particularly prominent during working memory and memory retrieval (episodic and semantic), and less prevalent during perception and perceptual priming tasks. This pattern is consistent with the idea that the prefrontal cortex is involved in working memory processes, such as monitoring, organization, and planning. However, some of the same prefrontal regions engaged by working tasks are also recruited by simple detection tasks that do not involve a maintenance component. Thus the prefrontal cortex is not devoted solely to working memory operations.

Regarding lateralization, prefrontal activations during language, semantic memory retrieval, and episodic memory encoding are usually left-lateralized, those during sustained attention and episodic retrieval are mostly right-lateralized, and those during working memory are typically bilateral.

With respect to distinctions between different prefrontal areas, ventrolateral regions (areas 45 and 47) are involved in selecting, comparing, or deciding on information held in short-term and long-term memory, whereas mid-dorsal regions (areas 9 and 46) are involved when several pieces of information in working memory need to be monitored and manipulated. Area 45/47 activations were found even in simple language tasks, while activations in areas 9/46 were associated with working memory and episodic encoding and retrieval. However, areas 9/46 were also activated during sustained attention tasks, which do not involve the simultaneous consideration of several pieces of information. See Cabeza et al, “Imaging Cognition II: An Empirical Review of 275 PET and fMRI Studies,” J. Cognitive Neurosci., vol. 12, pp. 1-47 (2000).

Humans restrain self-interest with moral and social values. They are the only species known to exhibit reciprocal fairness, which implies the punishment of other individuals' unfair behaviors, even if it hurts the punisher's economic self-interest. Reciprocal fairness has been demonstrated in the Ultimatum Game, where players often reject their bargaining partner's unfair offers. It has been shown that disruption of the right, but not the left, dorsolateral prefrontal cortex (DLPFC) by low-frequency repetitive transcranial magnetic stimulation substantially reduces subjects' willingness to reject their partners' intentionally unfair offers, which suggests that subjects are less able to resist the economic temptation to accept these offers. Importantly, however, subjects still judge such offers as very unfair, which indicates that the right DLPFC plays a key role in the implementation of fairness-related behaviors. See Knoch et al., “Diminishing Reciprocal Fairness by Disrupting the Right Prefrontal Cortex,” Science, vol. 314, pp. 829-832 (3 Nov. 2006).

Differences across tasks can be found in frontopolar (area 10), opercular (area 44), and dorsal (areas 6 and 8) prefrontal regions. Frontopolar activations were typical for episodic memory retrieval and problem-solving tasks. In the case of episodic retrieval, they are found for both retrieval success and retrieval mode, suggesting they are probably not related to performance level or task difficulty. Area 10 is involved in maintaining the mental set of episodic retrieval, but also has an involvement in problem-solving tasks. Activations in left area 44, which corresponds to Broca's area, were commonly found for reading, verbal working memory and semantic generation. Right area 44 is engaged by nonverbal episodic retrieval tasks. Area 6 plays a role in spatial processing (orientation of attention, space/motion perception and imagery), working memory, and motor-skill learning. Midline area 6 activations correspond to SMA and are common for silent reading tasks. Area 8 is involved in problem-solving tasks, possibly reflecting eye movements. See Cabeza et al, “Imaging Cognition II: An Empirical Review of 275 PET and fMRI Studies,” J. Cognitive Neurosci., vol. 12, pp. 1-47 (2000).

The frontopolar cortex has been shown to be active during the initial stages of learning, gradually disengaging over the course of learning. Frontopolar cortex activity specifically correlates with the amount of uncertainty remaining between multiple putative options that subjects are simultaneously tracking. The frontopolar cortex is also active whenever subjects depart from an a priori optimal option to check alternative ones. Thus the frontopolar cortex contribution to learning and exploration appears to be associated with maintaining and switching back and forth between multiple behavioral alternatives in search of optimal behavior. The frontopolar cortex has also been implicated in memory retrieval, relational reasoning, and multitasking behaviors. These subfunctions are thought to be integrated in the general function of contingently switching back and forth between independent tasks by maintaining distractor-resistant representations of postponed tasks during the performance of another task. For example, the frontopolar cortex is specifically activated when subjects suspend execution of an ongoing task set associated with a priori the largest expected future rewards in order to explore a possibly more-rewarding task set. See Keochlin et al., “Anterior Prefrontal Function and the Limits of Human Decision-Making,” Science, vol. 318, pp. 594-598 (26 Oct. 2007).

Activation of the medial prefrontal cortex and anterior paracingulate cortex indicate that a subject is thinking and acting on the beliefs of others, for example, either by guessing partner strategies or when comparing play with another human to play with a random device, such as a computer partner. Accordingly, these regions may be involved in intention detection, i.e., assessing the meaning of behavior from another agent. The tempo-parietal junction is also implicated in this function. Further, publication brand-related bias in the credibility of ambiguous news headlines is associated with activation changes in the medial prefrontal cortex. See Kenning et al., “Neuroeconomics: an overview from an economic perspective,” Brain Res. Bull., vol. 67, pp. 343-354 (2005).

In situations in which people gain some useful good (e.g., money, juice, or other incentive) by using judgment, activation can be observed in the so-called “reward areas” of the brain. Therefore, a “feeling” of approval or utility may correlate with the activation in the reward areas of the brain. Reward areas of the brain include the ventral striatum and the orbitofrontal prefrontal cortex-amygdala-nucleus accumbens circuit. Monetary payoffs induce activation in the nucleus accumbens. The nucleus accumbens is densely innervated by dopaminergic fibers originating from neurons in the midbrain. Sudden release of dopamine after an unexpected reward may lead to acceptance of risk. Accordingly, defects in the orbitofrontal cortex-amygdala-nucleus accumbens reward circuit may accompany extreme risk-seeking behavior. This reward system is also associated with the perception of utility of objects.

Cingulate Regions

Cingulate regions can be roughly classified as anterior (for example, areas 32 and 24), central (areas 23 and 31), and posterior (posterior area 31, retrosplenial). Posterior cingulate activations are consistently seen during successful episodic memory retrieval, as are other posterior midline activations (e.g., medial parietal, cuneus, precuneus). Anterior cingulate activations occur primarily in area 32 and are consistently found for S-R compatibility (Stroop test), working memory, semantic generation, and episodic memory tasks.

There are three main views of the anterior cingulate function: initiation, inhibitory, and motor. According to the initiation view, the anterior cingulate cortex is involved in “attention to action,” that is, in attentional processes required to initiate behavior. This is consistent with evidence that damage to this region sometimes produces akinetic mutism, that is, an almost complete lack of spontaneous motor or verbal behavior. This is also consistent with the involvement of this region in demanding cognitive tasks, such as working memory and episodic retrieval.

The inhibitory view postulates that the anterior cingulate is involved in suppressing inappropriate responses. This idea accounts very well not only for its involvement in the Stroop task, in which prepotent responses must be inhibited, but also in working memory, in which interference from previous trials must be controlled. The initiation and inhibition views are not incompatible: the anterior cingulate cortex may both initiate appropriate responses and suppress inappropriate ones. Moreover, these views share the idea that the anterior cingulate cortex plays an “active” role in cognition by controlling the operations of other regions, including the prefrontal cortex.

In contrast, the motor view conceptualizes the anterior cingulate as a more “passive” structure: it receives cognitive/motor “commands” from various regions (for example, prefrontal cortex), and “funnels” them to the appropriate motor system. This view assumes that different anterior cingulate regions are engaged, depending on whether responses are ocular, manual, or verbal. For example, due to its close connections to the auditory cortex, area 32 is assumed to play a role in vocalization and speech. This idea accounts for activations during tasks involving verbal materials, such as Stroop, semantic generation, and verbal episodic retrieval tasks. See Cabeza et al, “Imaging Cognition II: An Empirical Review of 275 PET and fMRI Studies,” J. Cognitive Neurosci., vol. 12, pp. 1-47 (2000).

Lying is associated with increased activity in several areas of the cortex, including the anterior cingulate cortex, the parietal cortex, and the superior frontal gyms. See Henig, “Looking for the Lie,” New York Times http://www.nytimes.com/2006/02/05/magazine/05lying.html?pagewanted=print (5 Feb. 2006).

Parietal Regions

Parietal regions are consistently activated during tasks involving attention, spatial perception and imagery, working memory, spatial episodic encoding, episodic retrieval, and skill learning. Medial parietal activations are frequently found during episodic memory retrieval. In general, lateral parietal activations relate either to spatial perception/attention or to verbal working memory storage. Parietal regions may be part of a dorsal occipito-parietal pathway involved in spatial perception, and/or part of a “posterior attention system” involved in disengaging spatial attention. These spatial views account for parietal activations during spatial tasks of perception, imagery, and episodic encoding, as well as for those during skill-learning tasks, which, typically, involve an important spatial component.

According to the working memory interpretation, parietal regions are involved in the storage of verbal information in working memory. This is consistent with evidence that left posterior parietal lesions can impair verbal short-term memory. See Cabeza et al, “Imaging Cognition II: An Empirical Review of 275 PET and fMRI Studies,” J. Cognitive Neurosci., vol. 12, pp. 1-47 (2000).

Temporal Regions

The temporal lobes can be subdivided into four broad regions: lateral (insula, 42, 22, 21, and 20), medial (areas 28, 34-36, and hippocampal regions), posterior (area 37), and polar (area 38). Area 38 is likely to have a very important role in cognition, for example, by linking frontal-lobe and temporal-lobe regions.

Lateral temporal activations are consistently found for language and semantic memory retrieval and are mostly left-lateralized. Spoken word-recognition tasks usually yield bilateral activations, possibly reflecting the auditory component of these tasks. The involvement of the left superior and middle temporal gyms (areas 22 and 21) in language operations is consistent with research on aphasic patients. Since area 21 is also consistently activated during semantic retrieval tasks—not only for verbal but also for nonverbal materials—it is possible that this area reflects semantic, rather than linguistic, operations. This is supported by the involvement of this region in object perception.

Medial-temporal lobe activations are repeatedly found for episodic memory encoding and nonverbal episodic memory retrieval. The involvement of medial temporal regions in episodic memory is consistent with lesion data. Based on PET data, encoding-related activations are more common in anterior hippocampal regions, whereas retrieval-related activations are more prevalent in posterior hippocampal regions, a pattern described as the hippocampal encoding/retrieval (HIPER) model. See Cabeza et al, “Imaging Cognition II: An Empirical Review of 275 PET and fMRI Studies,” J. Cognitive Neurosci., vol. 12, pp. 1-47 (2000).

Occipito-Temporal Regions

The engagement of temporo-occipital regions (areas 37, 19, 18, and 17) in cognitive tasks seems to be of two kinds: activations associated with perceiving and manipulating visuospatial information, and deactivations associated with perceptual priming. Visual processing along the ventral pathway is assumed to be organized hierarchically, with early image analyses engaging areas close to the primary visual cortex and higher-order object recognition processes involving more anterior areas. Consistent with this idea, activations in areas 18 and 19 occur for most visuospatial tasks, whereas activations in area 37 are associated with object processing. For example, area 37 activation is found when subjects perceive objects and faces, maintain images of objects in working memory, and intentionally encode objects. Perception-related occipital activations are enhanced by visual attention and they therefore can be expected during visual-attentional tasks, as well as during demanding visual-skill learning tasks (e.g., mirror reading).

Most activations in occipito-temporal regions occur during the processing of visual information coming from eyes (perception) or from memory (imagery), and weaken when the same information is repeatedly processed (priming). See Cabeza et al, “Imaging Cognition II: An Empirical Review of 275 PET and fMRI Studies,” J. Cognitive Neurosci., vol. 12, pp. 1-47 (2000).

Subcortical Regions

With respect to activations in the basal ganglia, the thalamus, and the cerebellum, basal ganglia activations were common during motor-skill learning, and the cerebellum was consistently activated in several different processes. Evolutionary, anatomical, neuropsychological, and functional neuroimaging evidence indicates that the cerebellum plays an important role in cognition. The cognitive role of the cerebellum has been related as motor-preparation, sensory acquisition, timing, and attention/anticipation. Each of these views can account for some cerebellar activations, but not for all of them. For example, the motor preparation view accounts well for activations during tasks involving motor responses, such as word production and conditioning, while the sensory-acquisition view can accommodate activations during perceptual tasks, such as smelling. The timing view accounts for activations during tasks involving relations between successive events, such as conditioning and skill learning, while the attention/anticipation view explains activations during attention and problem solving. See Cabeza et al, “Imaging Cognition II: An Empirical Review of 275 PET and fMRI Studies,” J. Cognitive Neurosci., vol. 12, pp. 1-47 (2000).

Mesolimbic Dopamine System

Activity in the striatum scales directly with the magnitude of monetary reward or punishment. The striatum is also involved in social decisions, above and beyond a financial component. The striatum also encodes abstract rewards such as positive feeling as a result of mutual cooperation. In addition, the caudate is activated in situations where a subject has an intention to trust another. Emotional processes are reliably associated with a series of structures including the striatum and caudate, and areas of the midbrain and cortex to which they project, such as the ventromedial prefrontal cortex, orbitofrontal cortex, and anterior cingulated cortex, as well as other areas such as the amygdala and the insula. Indeed, subjects with lesions in the ventromedial prefrontal cortex and having associated emotional deficits are impaired in performing gambling tasks. The anterior insula is associated with increased activation as unfairness or inequity of an offer is increased. Activation of the anterior insula predicts an Ultimatum Game player's decision to either accept or reject an offer, with rejections associated with significantly higher activation than acceptances. Activation of the anterior insula is also associated with physically painful, distressful, and/or disgusting stimuli. Thus, the anterior insula and associated emotion-processing areas may play a role in marking an interaction as aversive and undeserving of trust in the future. See Sanfey, “Social Decision-Making: Insights from Game Theory and Neuroscience,” Science, vol. 318, pp. 598-601 (26 Oct. 2007).

Activation in the ventral striatum is seen by fMRI when subjects provide a correct answer to a question, resulting in a reward. Similarly, a wrong answer and no payment results in a reduction in activity (i.e., oxygenated blood flow) to the ventral striatum. Moreover, activation of the reward centers of the brain including the ventral striatum over and above that seen from a correct response and reward is seen when a subject receives a reward that is known to be greater than that of a peer in the study. Thus, stimulation of the reward center appears to be linked not only to individual success and reward, but also to the success and rewards of others. See BBC news story “Men motivated by ‘superior wage,’” http://news.bbc.co.uk/1/hi/sci/tech/7108347.stm, (23 Nov. 2007).

In a multi-round trust game, reciprocity expressed by one player strongly predicts future trust expressed by their partner—a behavioral finding mirrored by neural responses in the dorsal striatum as measured by fMRI. Analyses within and between brains show two signals—one encoded by response magnitude, and the other by response timing. Response magnitude correlates with the “intention to trust” on the next play of the game, and the peak of these “intention to trust” responses shifts its time of occurrence by 14 seconds as player reputations develop. This temporal transfer resembles a similar shift of reward prediction errors common to reinforcement learning models, but in the context of a social exchange. See King-Casas et al., “Getting to Know You: Reputation and Trust in a Two-Person Economic Exchange,” Science, vol. 308, pp. 78-83 (1 Apr. 2005).

Activity in the head of the caudate nucleus is associated with the processing of information about the fairness of a social partner's decision and the intention to repay with trust, as measured by hyperscan-fMRI. See Kenning et al., “Neuroeconomics: an overview from an economic perspective,” Brain Res. Bull., vol. 67, pp. 343-354 (2005).

Activation of the insular cortex is associated with the perception of bodily needs, providing direction to motivated behaviors. For example, imaging studies have shown activation of the insula in addicts with cue-induced drug craving, and activation of the insular cortex has been associated with subjective reports of drug craving. See Contreras et al., “Inactivation of the Interoceptive Insula Disrupts Drug Craving and Malaise Induced by Lithium,” Science, vol. 318, pp. 655-658 (26 Oct. 2007).

Visual Cortex

The visual cortex is located in and around the calcarine fissure in the occipital lobe. In one visual cortex study, subjects were shown two patterns in quick succession. The first appeared for just 15 milliseconds, too fast to be consciously perceived by the viewer. By examining fMRI images of the brain, a specific image that had been flashed in front of the subjects could be identified. The information was perceived in the brain even if the subjects were not consciously aware of it. The study probed the part of the visual cortex that detects a visual stimulus, but does not perceive it. It encodes visual information that the brain does not process as “seen.” See “Mind-reading machine knows what you see,” NewScientist.com http://www.newscientist.com/article.ns?id=dn7304&feedId=online-news_rss20 (25 Apr. 2005).

Hippocampus

Activation of the hippocampus can modulate eating behaviors linked to emotional eating and lack of control in eating. Activation of brain areas known to be involved in drug craving in addicted subjects, such as the orbitofrontal cortex, hippocampus, cerebellum, and striatum, suggests that similar brain circuits underlie the enhanced motivational drive for food and drugs seen in obese and drug-addicted subjects. See Wang et al., “Gastric stimulation in obese subjects activates the hippocampus and other regions involved in brain reward circuitry,” PNAS, vol. 103, pp. 15641-45 (2006).

Surrogate Markers of Mental State

Surrogate markers of mental state may include indicators of attention, approval, disapproval, recognition, cognition, memory, trust, or the like in response to a stimulus, other than measurement of brain activity associated with the stimulus. These surrogate markers of mental state may be used as a user-health test function, e.g., user-health test function 130.

Examples of surrogate markers may include a skin response to a stimulus; a face pattern indicative of approval, disapproval, or emotional state; eye movements or pupil movements indicating visual attention to an object; voice stress patterns indicative of a mental state, or the like. Surrogate markers may be used in conjunction with brain activity measurements for higher confidence in a predictive or interpretational outcome. For example, brain activation of the caudate nucleus in combination with calm voice patterns may increase confidence in a predictor of trust between a subject and a stimulus. Conversely, conflict between brain activity and a surrogate marker may decrease confidence in a predictive or interpretational outcome. For example, a pattern of activation of the insula diagnostic for fear, together with a visual face image showing a smile may decrease the level of confidence that the subject is truly frightened by a stimulus.

For example, emotion links to cognition, motivation, memory, consciousness, and learning and developmental systems. Affective communication depends on complex, rule-based systems with multiple channels and redundancy built into the exchange system, in order to compensate if one channel fails. Channels can include all five senses: for example, increased heart-rate or sweating may show tension or agitation and can be heard, seen, touched, smelt or tasted. Emotional exchanges may be visible displays of body tension or movement, gestures, posture, facial expressions or use of personal space; or audible displays such as tone of voice, choice of pitch contour, choice of words, speech rate, etc. Humans also use touch, smell, adornment, fashion, architecture, mass media, and consumer products to communicate our emotional state. Universals of emotion that cross cultural boundaries have been identified, and cultural differences have also been identified. For example ‘love’ is generally categorized as a positive emotion in Western societies, but in certain Eastern cultures there is also a concept for ‘sad love.’ Accordingly, universal emotional triggers may be used to transcend cultural barriers.

When communicating with computers, people often treat new media as if they were dealing with real people. They often follow complex social rules for interaction and modify their communication to suit their perceived conversation partner. Much research has focused on the use of facial actions and ways of coding them. Speech recognition systems have also attracted attention as they grow in capability and reliability, and can recognize both verbal messages conveyed by spoken words, and non verbal messages, such as those conveyed by pitch contours.

System responses and means of expressing emotions also vary. Innovative prototypes are emerging designed to respond indirectly, so the user is relatively unaware of the response: for example by adaptation of material, such as changing pace or simplifying or expanding content. Other systems use text, voice technology, visual agents, or avatars to communicate. See Axelrod et al., “Smoke and Mirrors: Gathering User Requirements for Emerging Affective Systems,” 26th Int. Conf. Information Technology Interfaces/TI 2004, Jun. 7-10, 2004, Cavtat, Croatia, pp. 323-328.

Skin Response

Mental state may be determined by detection of a skin response associated with a stimulus. One skin response that may correlate with mental state and/or brain activity is galvanic skin response (GSR), also known as electrodermal response (EDR), psychogalvanic reflex (PGR), or skin conductance response (SCR). This is a change in the electrical resistance of the skin. There is a relationship between sympathetic nerve activity and emotional arousal, although one may not be able to identify the specific emotion being elicited. The GSR is highly sensitive to emotions in some people. Fear, anger, startle response, orienting response, and sexual feelings are all among the emotions which may produce similar GSR responses. GSR is typically measured using electrodes to measure skin electrical signals.

For example, an Ultimate Game study measured skin-conductance responses as a surrogate marker or autonomic index for affective state, and found higher skin conductance activity for unfair offers, and as with insular activation in the brain, this measure discriminated between acceptances and rejections of these offers. See Sanfey, “Social Decision-Making: Insights from Game Theory and Neuroscience,” Science, vol. 318, pp. 598-601 (26 Oct. 2007). Other skin responses may include flushing, blushing, goose bumps, sweating, or the like.

Face Pattern Recognition

Mental state may also be determined by detection of facial feature changes associated with a stimulus, via pattern recognition, emotion detection software, face recognition software, or the like.

For example, an emotional social intelligence prosthetic device has been developed that consists of a camera small enough to be pinned to the side of a pair of glasses, connected to a hand-held computer running image recognition software plus association software that can read the emotions these images show. If the wearer seems to be failing to engage his or her listener, the software makes the hand-held computer vibrate. The association software can detect whether someone is agreeing, disagreeing, concentrating, thinking, unsure, or interested, just from a few seconds of video footage. Previous computer programs have detected the six more basic emotional states of happiness, sadness, anger, fear, surprise and disgust. The system can detect a sequence of movements beyond just a single facial expression. The association program is based on a machine-learning algorithm that was trained by showing it more than 100 8-second video clips of actors expressing particular emotions. The software picks out movements of the eyebrows, lips and nose, and tracks head movements such as tilting, nodding, and shaking, which it then associates with the emotion the actor was showing. When presented with fresh video clips, the software gets people's emotions right 90 percent of the time when the clips are of actors, and 64 percent of the time on footage of ordinary people. See “Device warns you if you're boring or irritating,” NewScientist http://www.newscientist.com/article/mg19025456.500-device-warns-you-if-youre-boring-or-irritating.html (29 Mar. 2006).

In another approach, an imager, such as a CCD camera, may observe expressed features of the user. For example, the imager may monitor pupil dilation, eye movement, expression, or a variety of other expressive indicators. Such expressive indicators may indicate a variety of emotional, behavioral, intentional, or other aspects of the user. For example, in one approach, systems have been developed for identifying an emotional behavior of a person based upon selected expressive indicators. Similarly, eye movement and pupil dilation may be correlated to truthfulness, stress, or other user characteristics.

Eye Movement Analysis

Eye movement or pupil movement can be tested, for example, by measuring user pupil and/or eye movements, perhaps in relation to items on a display. For example, a user's eye movement to a part of the screen containing an advertisement may be of interest to an advertiser for purposes of advertisement placement or determining advertising noticeability and/or effectiveness within a computerized game world. For example, knowing that a user's eyes have been attracted by an advertisement may be of interest to an advertiser. For example, a merchant may be interested in measuring whether a user notices a virtual world avatar having particular design characteristics. If the user exhibits eye movements toward the avatar on a display, then the merchant may derive a mental state from repeated eye movements vis a vis the avatar, or the merchant may correlate eye movements to the avatar with other physiological activity data such as brain activation data indicating a mental state such as brand preference, approval or reward.

In another embodiment, a smart camera may be used that can capture images of a user's eyes, process them and issue control commands within a millisecond time frame. Such smart cameras are commercially available (e.g., Hamamatsu's Intelligent Vision System; http://jp.hamamatsu.com/en/product_info/index.html). Such image capture systems may include dedicated processing elements for each pixel image sensor. Other camera systems may include, for example, a pair of infrared charge coupled device cameras to continuously monitor pupil size and position as a user watches a visual target moving, e.g., forward and backward. This can provide real-time data relating to pupil accommodation relative to objects on a display, which information may be of interest to an entity 170 (e.g., http://jp.hamamatsu.com/en/rd/publication/scientific_american/common/pdf/scientific_0608.pdf).

Eye movement and/or pupil movement may also be measured by video-based eye trackers. In these systems, a camera focuses on one or both eyes and records eye movement as the viewer looks at a stimulus. Contrast may be used to locate the center of the pupil, and infrared and near-infrared non-collumnated light may be used to create a corneal reflection. The vector between these two features can be used to compute gaze intersection with a surface after a calibration for a subject.

Two types of eye tracking techniques include bright pupil eye tracking and dark pupil eye tracking. Their difference is based on the location of the illumination source with respect to the optics. If the illumination is coaxial with the optical path, then the eye acts as a retroreflector as the light reflects off the retina, creating a bright pupil effect similar to red eye. If the illumination source is offset from the optical path, then the pupil appears dark.

Bright Pupil tracking creates greater iris/pupil contrast allowing for more robust eye tracking with all iris pigmentation and greatly reduces interference caused by eyelashes and other obscuring features. It also allows for tracking in lighting conditions ranging from total darkness to very bright light. However, bright pupil techniques are not recommended for tracking outdoors as extraneous IR sources may interfere with monitoring.

Eye tracking configurations can vary; in some cases the measurement apparatus may be head-mounted, in some cases the head should be stable (e.g., stabilized with a chin rest), and in some cases the eye tracking may be done remotely to automatically track the head during motion. Most eye tracking systems use a sampling rate of at least 30 Hz. Although 50/60 Hz is most common, many video-based eye trackers run at 240, 350 or even 1000/1250 Hz, which is recommended in order to capture the detail of the very rapid eye movements during reading, or during studies of neurology.

Eye movements are typically divided into fixations, when the eye gaze pauses in a certain position, and saccades, when the eye gaze moves to another position. A series of fixations and saccades is called a scanpath. Most information from the eye is made available during a fixation, not during a saccade. The central one or two degrees of the visual angle (the fovea) provide the bulk of visual information; input from larger eccentricities (the periphery) generally is less informative. Therefore the locations of fixations along a scanpath indicate what information loci on the stimulus were processed during an eye tracking session. On average, fixations last for around 200 milliseconds during the reading of linguistic text, and 350 milliseconds during the viewing of a scene. Preparing a saccade towards a new goal takes around 200 milliseconds.

Scanpaths are useful for analyzing cognitive intent, interest, and salience. Other biological factors (some as simple as gender) may affect the scanpath as well. Eye tracking in human-computer interaction typically investigates the scanpath for usability purposes, or as a method of input in gaze-contingent displays, also known as gaze-based interfaces.

There are two primary components to most eye tracking studies: statistical analysis and graphic rendering. These are both based mainly on eye fixations on specific elements. Statistical analyses generally sum the number of eye data observations that fall in a particular region. Commercial software packages may analyze eye tracking and show the relative probability of eye fixation on each feature on an avatar. This allows for a broad analysis of which avatar elements received attention and which ones were ignored. Other behaviors such as blinks, saccades, and cognitive engagement can be reported by commercial software packages. Statistical comparisons can be made to test, for example, competitors, prototypes or subtle changes to an avatar. They can also be used to compare participants in different demographic groups. Statistical analyses may quantify where users look, sometimes directly, and sometimes based on models of higher-order phenomena (e.g., cognitive engagement).

In addition to statistical analysis, it is often useful to provide visual depictions of eye tracking results. One method is to create a video of an eye tracking testing session with the gaze of a participant superimposed upon it. This allows one to effectively see through the eyes of the consumer during interaction with a target medium. Another method graphically depicts the scanpath of a single participant during a given time interval. Analysis may show each fixation and eye movement of a participant during a search on a virtual shelf display of breakfast cereals, analyzed and rendered with a commercial software package. For example, a different color may represent one second of viewing time, allowing for a determination of the order in which products are seen. Analyses such as these may be used as evidence of specific trends in visual behavior.

A similar method sums the eye data of multiple participants during a given time interval as a heat map. A heat map may be produced by a commercial software package, and shows the density of eye fixations for several participants superimposed on the original stimulus, for example, an avatar on a magazine cover. Red and orange spots represent areas with high densities of eye fixations. This allows one to examine which regions attract the focus of the viewer.

Commercial eye tracking applications include web usability, advertising, sponsorship, package design and automotive engineering. Eye tracking studies may presenting a target stimulus to a sample of consumers while an eye tracker is used to record the activity of the eye. Examples of target stimuli may include avatars in the context of websites, television programs, sporting events, films, commercials, magazines, newspapers, packages, shelf displays, consumer systems (ATMs, checkout systems, kiosks), and software. The resulting data can be statistically analyzed and graphically rendered to provide evidence of specific visual patterns. By examining fixations, saccades, pupil dilation, blinks, and a variety of other behaviors, researchers can determine a great deal about the effectiveness of a given avatar in a given medium or associated with a given product.

A prominent field of eye tracking research is web usability. While traditional usability techniques are often quite powerful in providing information on clicking and scrolling patterns, eye tracking offers the ability to analyze user interaction between the clicks. This provides insight into which features are the most eye-catching, which features cause confusion, and which ones are ignored altogether. Specifically, eye tracking can be used to assess impressions of an avatar in the context of search efficiency, branding, online advertisement, navigation usability, overall design, and/or many other site components. Analyses may target an avatar on a prototype or competitor site in addition to the main client site.

Eye tracking is commonly used in a variety of different advertising media. Commercials, print ads, online ads, and sponsored programs are all conducive to analysis with eye tracking technology. Analyses may focus on visibility of a target avatar, product, or logo in the context of a magazine, newspaper, website, virtual world, or televised event. This allows researchers to assess in great detail how often a sample of consumers fixates on the target avatar, logo, product, or advertisement. In this way, an advertiser can quantify the success of a given campaign in terms of actual visual attention.

Eye tracking also provides avatar designers with the opportunity to examine the visual behavior of a consumer while interacting with a target avatar. This may be used to analyze distinctiveness, attractiveness and the tendency of the avatar to be chosen for recognition and/or purchase. Eye tracking can be used while the target avatar is in the prototype stage. Prototype avatars can be are tested against each other and against competitors to examine which specific elements are associated with high visibility and/or appeal.

Another application of eye tracking research is in the field of automotive design. Eye tracking cameras may be integrated into automobiles to provide the vehicle with the capacity to assess in real-time the visual behavior of the driver. The National Highway Traffic Safety Administration (NHTSA) estimates that drowsiness is the primary causal factor in 100,000 police-reported accidents per year. Another NHTSA study suggests that 80% of collisions occur within three seconds of a distraction. By equipping automobiles with the ability to monitor drowsiness, inattention, and cognitive engagement driving safety could be dramatically enhanced. Lexus® claims to have equipped its LS 460 automobile with the first driver monitor system in 2006, providing a warning if the driver takes his or her eye off the road.

Eye tracking is also used in communication systems for disabled persons, allowing the user to speak, mail, surf the web and so on with only the eyes as tool. Eye control works even when the user has involuntary body movement as a result of cerebral palsy or other disability, and/or when the user wears glasses.

Eye movement or pupil movement may be gauged from a user's interaction with an application.

An example of a measure of pupil movement may be an assessment of the size and symmetry of a user's pupils before and after a stimulus, such as light or focal point. In one embodiment, where the user interacts with a head mounted display, the display may include image capturing features that may provide information regarding expressive indicators. Such approaches have been described in scanned-beam display systems such as those found in U.S. Pat. No. 6,560,028.

Voice Stress Analysis

Voice stress analysis (VSA) technology records psycho-physiological stress responses that are present in the human voice when a person experiences a psychological stress in response to a stimulus. Psychological stress may be detected as acoustic modifications in the fundamental frequency of a speaker's voice relative to normal frequency modulation of the vocal signal between 8-14 Hz during speech in an emotionally neutral situation. In situations involving a stress response, the 8-14 Hz modulation may decrease as the muscles surrounding the vocal cords contract in response to the reaction.

VSA typically records an inaudible component of human voice, commonly referred to as the Lippold Tremor. Under normal circumstances, the laryngeal muscles are relaxed, producing recorded voice at approximately 12 Hz. Under stress however, the tensed laryngeal muscles produce voice significantly lower than normal. The higher the stress, the lower down the Hertz scale voice waves are produced. One application for VSA is in the detection of deception.

Dektor Counterintelligence manufactured the PSE 1000, an analog machine that was later replaced by the PSE 2000. The National Institute Of Truth Verification (NITV) then produced and marketed a digital application based on the McQuiston-Ford algorithm. The primary commercial suppliers are Dektor (PSE5128-software); Diogenes (Lantern-software); NITV (CVSA Software); and Baker (Baker-software).

VSA is distinctly different from LVA (Layered Voice Analysis). LVA is used to measure different components of voice, such as pitch and tone. LVA is available in the form of hand-held devices and software. LVA produces readings such as ‘love,’ excitement, and fear.

One example of a commercially available layered voice analysis system is the SENSE system, sold by Nemesysco Ltd (Natania, Israel). SENSE can analyze different layers within the voice, using multiple parameters to analyze each speech segment. SENSE can detect various cognitive states, such as whether a subject is excited, confused, stressed, concentrating, anticipating a response, or unwillingly sharing information. The technology also can provide an in-depth view of the subject's range of emotions, including those relating to love. SENSE technology can be further utilized to identify psychological issues, mental illness, and other behavioral patterns. The LVA technology is the security version of the SENSE technology, adapted to identify the emotional situations a subject is expected to have during formal/security investigations.

The SENSE technology is made up of 4 sub-processes:

1. The vocal waveform is analyzed to measure the presence of local micro-high frequencies, low frequencies, and changes in their presence within a single voice sample.

2. A precise frequency spectrum of the vocal input is sampled and analyzed.

3. The parameters gathered by the previous steps are used to create a baseline profile for the subject.

4. The new voice segments to be tested are compared with the subject's baseline profile, and the analysis is generated.

This input can be further processed by statistical learning algorithms to predict the probability of a deceptive or fraudulent sentence in a subject's speech. Another layer that is used in certain applications evaluates the conversation as a whole, and produces a final risk or QA value.

The SENSE technology can detect the following emotional and cognitive states:

Excitement Level: Each of us becomes excited (or depressed) from time to time. SENSE compares the presence of the Micro-High-frequencies of each sample to the basic profile to measure the excitement level in each vocal segment.

Confusion Level: Is your subject sure about what he or she is saying? SENSE technology measures and compares the tiny delays in a subject's voice to assess how certain he or she is.

Stress Level: Stress may include the body's reaction to a threat, either by fighting the threat, or by fleeing. However, during a spoken conversation neither option may be available. The conflict caused by this dissonance affects the micro-low-frequencies in the voice during speech.

Thinking Level: How much is your subject trying to find answers? Might he or she be “inventing” stories?

S.O.S.: (Say Or Stop)—Is your subject hesitating to tell you something? Concentration Level: Extreme concentration might indicate deception.

Anticipation Level: Is your subject anticipating your responses according to what he or she is telling you?

Embarrassment Level: Is your subject feeling comfortable, or does he feel some level of embarrassment regarding what he or she is saying?

Arousal Level: What triggers arousal in the subject? Is he or she interested in an object? Aroused by certain visual stimuli?

Deep Emotions: What long-standing emotions does a subject experience? Is he or she “excited” or “uncertain” in general?

SENSE's “Deep” Technology: Is a subject thinking about a single topic when speaking, or are there several layers to a response (e.g., background issues, something that may be bothering him or her, planning, or the like). SENSE technology can detect brain activity operating at a pre-conscious level.

The speaking mechanism is one of the most complicated procedures the human body is capable of. First, the brain has to decide what should be said, then air is pushed from the lungs upward to the vocal cords, that must vibrate to produce the main frequency. Now, the vibrated air arrives to the mouth.

The tongue, the lips, the teeth, and the nose space turns the vibrated air into the sounds that we recognize as phrases. The brain is closely monitoring all these events, and listens to what comes out; if we speak too softly, too loudly, and if it is understandable to a listener. SENSE Technology ignores what your subject is saying, and focuses only on what the brain is broadcasting.

Humans, unlike other mammals, are capable of predicting or imagining the future. Most people can tell whether or not a certain response will cause them pleasure or pain. Lying is not a feeling, it is a tool. The feeling structure around it will be the one causing us to lie, and understanding the differences is crucial for making an analysis.

The SENSE technology differentiates among 5 types of lies:

1. Jokes—Jokes are not so much lies as they are untruths, used to entertain. No long gain profit or loss will be earned from it, and usually, little or no extra feelings will be involved.

2. White Lies—You know you don't want to say the truth, as it may hurt someone else. White lies are lies, but the teller usually experiences little stress or guilt.

3. Embarrassment Lies—Same as for white lies, but this time directed internally. Nothing will be lost except the respect of the listener, most likely for the short term.

4. Offensive Lies—This is a unique lie, for it's intention is to gain something extra that could not be gained otherwise.

5. Defensive Lie—The common lie to protect one's self

The SENSE technology IS the old “Truster” technology, with several additions and improvements. The old Truster was all about emotions in the context of Truth/Lie; SENSE looks at emotions in general.

When people get sexually aroused or feel “in love,” the pupils get wider, the lips get reddish, the skin of the face gets red. The voice changes too. Increased excitement makes the whole voice higher and more concentrated. The SENSE technology can detect the increased excitement and the associated heightened concentration and anticipation.

While each of the above described approaches to providing expressive indicators has been described independently, in some approaches, a combination of two or more of the above described approaches may be implemented to provide additional information that may be useful in evaluating user behavior and/or mental state.

Following are a series of flowcharts depicting implementations. For ease of understanding, the flowcharts are organized such that the initial flowcharts present implementations via an example implementation and thereafter the following flowcharts present alternate implementations and/or expansions of the initial flowchart(s) as either sub-component operations or additional component operations building on one or more earlier-presented flowcharts. Those having skill in the art will appreciate that the style of presentation utilized herein (e.g., beginning with a presentation of a flowchart(s) presenting an example implementation and thereafter providing additions to and/or further details in subsequent flowcharts) generally allows for a rapid and easy understanding of the various process implementations. In addition, those skilled in the art will further appreciate that the style of presentation used herein also lends itself well to modular and/or object-oriented program design paradigms.

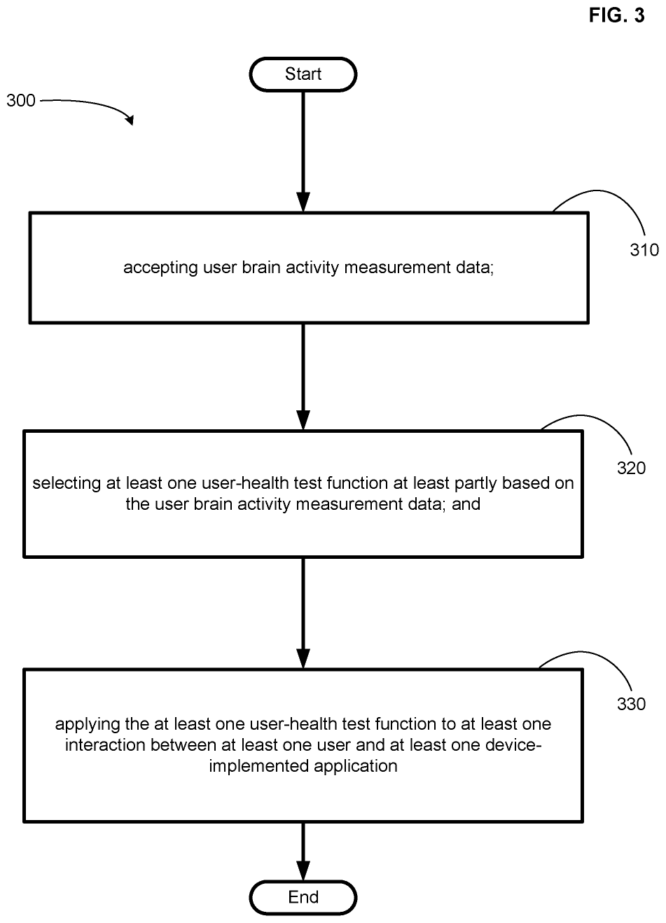

FIG. 3 illustrates an operational flow 300 representing example operations related to computational user-health testing. In FIG. 3 and in following figures that include various examples of operational flows, discussion and explanation may be provided with respect to the above-described system environments of FIGS. 1-2, and/or with respect to other examples and contexts. However, it should be understood that the operational flows may be executed in a number of other environments and contexts, and/or in modified versions of FIG. 12. Also, although the various operational flows are presented in the sequence(s) illustrated, it should be understood that the various operations may be performed in other orders than those which are illustrated, or may be performed concurrently.

After a start operation, operation 310 shows accepting user brain activity measurement data. For example, device 102, brain activity measurement unit 186 and/or user-health test function selection module 138 may accept user brain activity measurement data. In one embodiment, user-health test function selection module 138 may accept functional brain activity mapping data. Such data may be accepted in real time during an interaction of a user 190 with an application 104 operable on a device 102. The brain activity mapping data, as discussed above, may indicate a mental state of the user 190 and/or the data may indicate brain function deficiency in the user 190, such as attention deficit, stroke, or the like, localized to a particular brain area. User-health test function selection module 138 may, on the basis of accepted brain activity measurement data, match specific brain activity with an appropriate user-health test function in order to confirm a suspected diagnosis, obtain further data regarding a possible symptom or condition, rule out a possible diagnosis, or the like.

Brain activity measurement data may include various types of brain activity data, including but not limited to electroencephalography data (EEG), magnetoencephalography data (MEG), functional magnetic resonance imaging data (fMRI), functional near infra-red imaging data (fNIR), single proton emission computed tomography (SPECT). For example, brain activity measurement data from a user 190 with a particular health concern may indicate an anomalous pattern of activity in a certain brain area, for example the motor cortex, indicating a possible problem with the user 190's motor function. By referring to a database or look-up chart, a user-health test function selection module 138 may match motor cortex activity pattern data with one or more appropriate user-health test functions that can further test the motor function of user 190, e.g., a motor skill test function such as a hand-eye coordination challenge in a computer game.

Operation 320 depicts selecting at least one user-health test function at least partly based on the user brain activity measurement data. For example, device 102 and/or user-health test function selection module 138 may select at least one user-health test function at least partly based on user brain activity measurement data. In one embodiment, user-health test function selection module 138 may accept brain activity measurement data indicating brain activity in an area of the brain responsible for memory. Accordingly, the user-health test function selection module 138 may select a memory test function in order to further examine the memory function of user 190 with Alzheimer's disease. In another example, device 102 may receive brain activity measurement data indicating activation of an attention area of the brain in a user suspected of having an attention deficit disorder. An alertness test function and/or an attention test function may then be selected based on the brain activity measurement data. An alertness test function and/or an attention test function may be contained within a specific user-health test function set 198, including various alertness or attention test functions described below, such as a reaction time test function and/or a test of a user's ability to say a series of numbers forward and backwards.

Alternatively, user-health test function selection may be carried out based on a best-fit analysis of the brain activity measurement data together with potential user-health test functions. Various best-fit analysis methods are known in the art and can be employed or adapted by one of skill in the art (see, for example, Zhou G., U.S. Pat. No. 6,999,931 “Spoken dialog system using a best-fit language model and best-fit grammar”).

Operation 330 depicts applying the at least one user-health test function to at least one interaction between at least one user and at least one device-implemented application. For example, the at least one device 102, user-health test function selection module 138, and/or user-health test function unit 140 may apply a particular user-health test function 130 such as a pointing device manipulation test function, for example, based on brain activity measurement data indicating possible Parkinson's disease progression. The pointing device manipulation test function may be applied to an interaction between the user 190 and a game operable on the device 102, for example. Alternatively, the pointing device manipulation test function may be applied to an interaction between the user 190 and a diagnostic program, for example, a program specifically designed to test for Parkinson's disease progression.

Another example of applying the at least one user-health test function to at least one interaction between at least one user and at least one device-implemented application is applying a selected hearing test function to an interaction between a user and a music-playing device, video-playing device, or other personal entertainment device that emits sound. In this case, the device-implemented application can be a media player for playing music or movies, or the like. Similarly, a selected vision test function may be applied by the at least one device 102 to an interaction between a user and a media player application that, for example, displays a photograph or movie on a computer screen or other monitoring device.

System 100 and/or the at least one device 102 may include an application 104 that is operable on the at least one device 102, optionally for performing a primary function unrelated to user-health testing. For example, an online computer game may be operable as an application 104 on a personal computing device through a network 192. Thus the at least one application 104 may reside on the at least one device 102, or the at least one application 104 may not reside on the at least one device 102 but instead be operable on the at least one device 102 from a remote location, for example, through a network or other link.

User-health data signals such as brain activity measurement data may first be encoded and/or represented in digital form (i.e., as digital data), prior to the assignment to at least one memory. For example, a digitally-encoded representation of user brain activity measurement data may be stored in a local memory, or may be transmitted for storage in a remote memory.

Thus, an operation may be performed relating either to a local or remote storage of the digital data, or to another type of transmission of the digital data. Operations also may be performed relating to accessing, querying, processing, recalling, or otherwise obtaining the digital data from a memory, including, for example, receiving a transmission of the digital data from a remote memory. Accordingly, such operation(s) may involve elements including at least an operator (e.g., either human or computer) directing the operation, a transmitting computer, and/or a receiving computer.

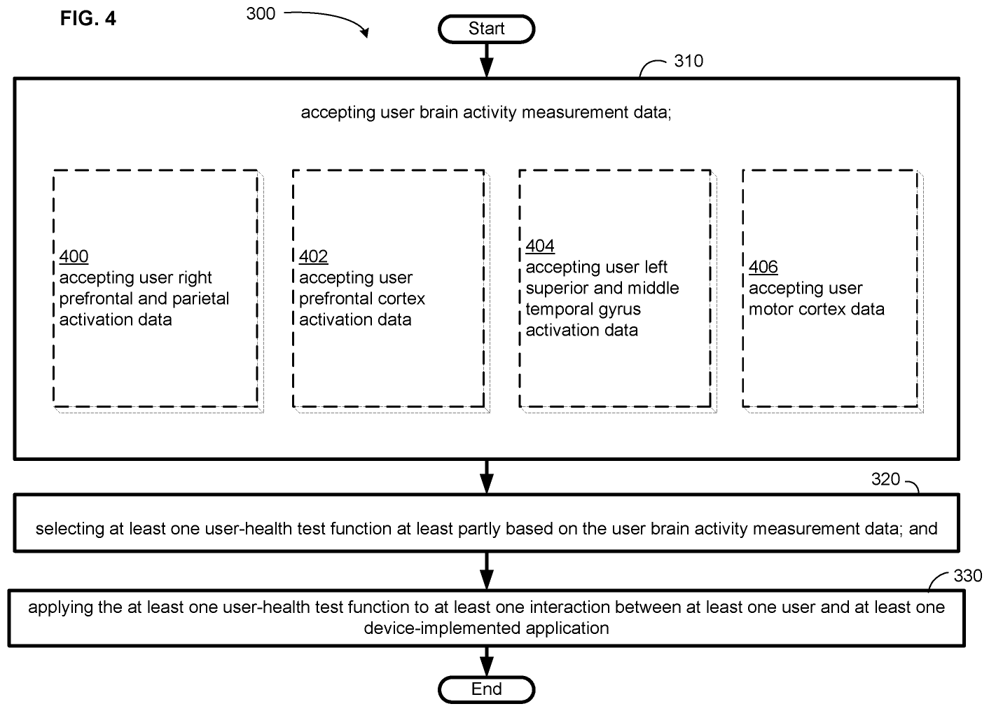

FIG. 4 illustrates alternative embodiments of the example operational flow 300 of FIG. 3. FIG. 4 illustrates example embodiments where the accepting operation 310 may include at least one additional operation. Additional operations may include operation 400, 402, 404, and/or operation 406.

Operation 400 depicts accepting user right prefrontal and parietal activation data. For example, the at least one device 102, user-health test function selection module 138, and/or brain activity measurement unit 186 may accept user right prefrontal and parietal activation data. In one embodiment, brain activity measurement unit 186 may accept data indicating an activation of the right prefrontal lobe and parietal areas of the brain in a user 190, for example, during an interaction with a social networking website such as facebook.com. In an example in which the user 190 may have an attention deficit disorder, the brain activity measurement data may be matched with an attention test function, for example, by user-health test function selection module 138.

Operation 402 depicts accepting user prefrontal cortex activation data. For example, the at least one device 102, user-health test function selection module 138, and/or brain activity measurement unit 186 may accept user prefrontal cortex activation data. In one embodiment, user prefrontal cortex activation data may be obtained by a functional infra-red imaging headset operational during an interaction between a user 190 and, for example, an online game. Where the brain activity measurement data indicate activation of areas of the brain involved in memory, such as the prefrontal cortex, a memory test function may be selected by, for example, user-health test function selection module 138.

Operation 404 depicts accepting user left superior and middle temporal gyms activation data. For example, the at least one device 102, user-health test function selection module 138, and/or brain activity measurement unit 186 may accept user left superior and middle temporal gyms activation data. For example, left superior and middle temporal gyms activation data indicating activity in the speech centers of the user 190 may be accepted by device 102 from brain activity measurement unit 186. At some time later, perhaps at a predetermined time or when triggered by a user action such as initiating an interaction with application 104, device 102 and/or user-health test function selection module 138 may access the user left superior and middle temporal gyms activation data and select a corresponding user-health test function, such as a speech test function, and/or a complementary user-health test function, such as a hearing test function.

Operation 406 depicts accepting user motor cortex data. For example, the at least one device 102, user-health test function selection module 138, and/or brain activity measurement unit 186 may accept user motor cortex data. In one embodiment, user-health test function selection module 138 may accept user motor cortex data during interaction with, for example, a game 106 requiring physical interaction, such as a Wii Fit game. In this example, anomalous motor cortex activity may be recognized by user-health test function selection module 138, causing it to initiate a motor skill user-health test function, perhaps as a subroutine within a Wii Fit application 104. Such a motor skill user-health test function may serve to evaluate the nature and/or extent of any impairment that the user 190 may be experiencing.

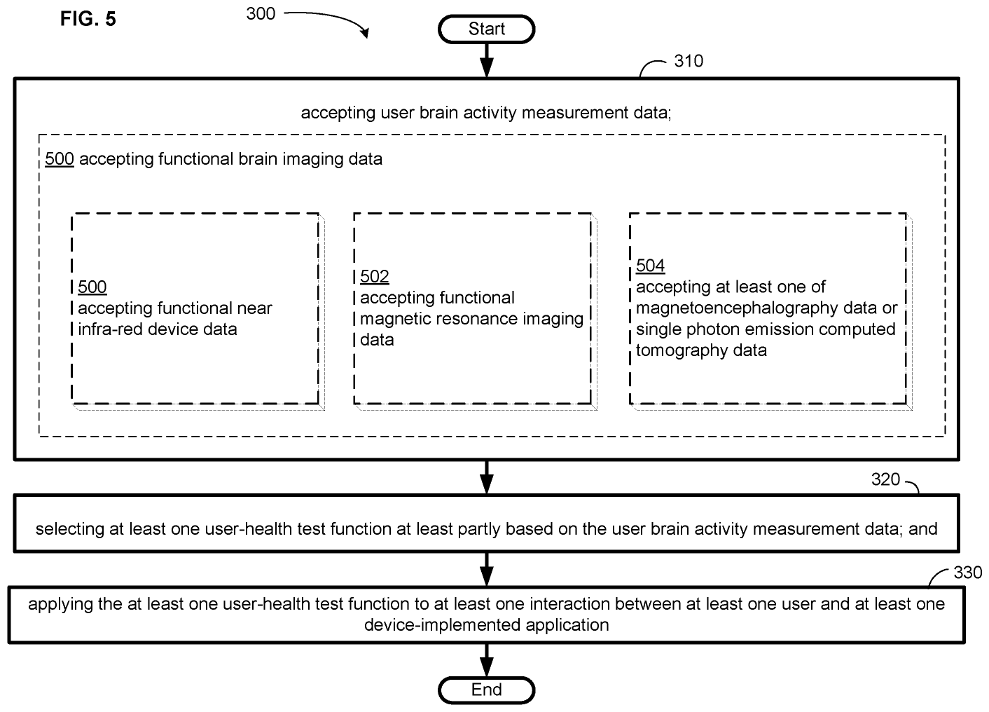

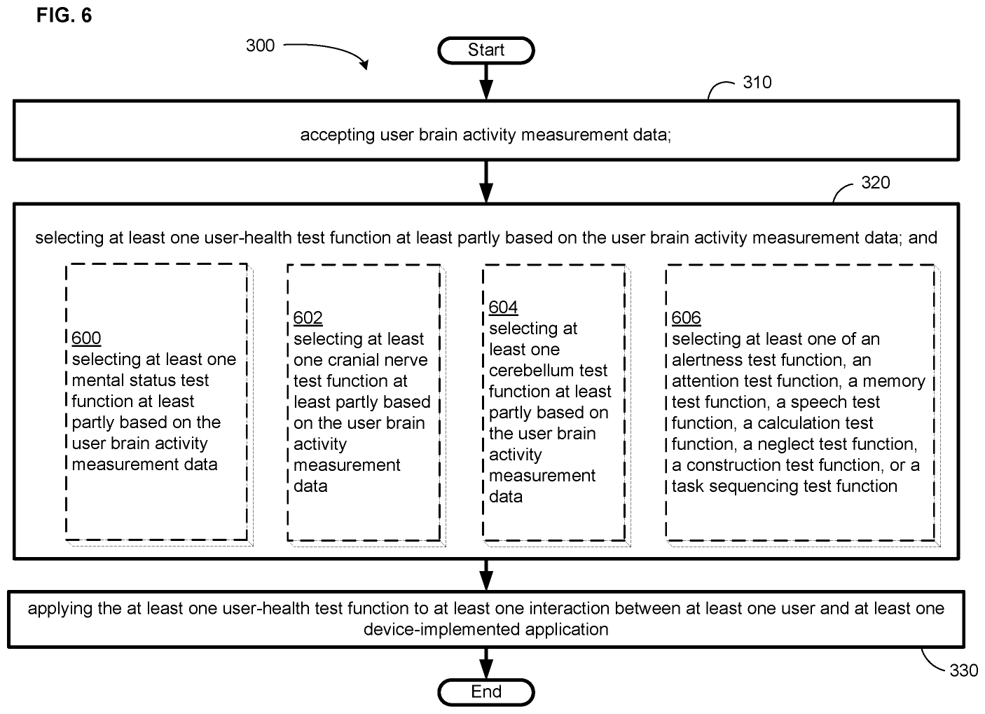

FIG. 5 illustrates alternative embodiments of the example operational flow 300 of FIG. 3. FIG. 5 illustrates example embodiments where the accepting operation 310 may include at least one additional operation. Additional operations may include operation 500, 502, and/or operation 504.

Operation 500 depicts accepting functional brain imaging data. For example, the at least one device 102, user-health test function selection module 138, and/or brain activity measurement unit 186 may accept functional brain imaging data. In one embodiment, the at least one device 102 may accept positron emission tomography data of a user's brain activity. Functional brain imaging data may include any imaging data that is a measure of neuronal activity in the brain. For example, electrical activity, magnetic activity, and metabolic activity associated with neuronal signaling are all kinds of functional brain imaging. Functional brain imaging may be carried out by, for example, a near infra-red imaging device, a magnetic resonance imaging device, a magnetoencephalography device, a positron emission tomography device, or the like. In some embodiments, functional brain imaging data may reflect blood oxygenation in one or more areas of the brain.

Operation 502 depicts accepting functional near infra-red device data. For example, the at least one device 102, user-health test function selection module 138, and/or brain activity measurement unit 186 may accept functional near infra-red device data. In one embodiment, user-health test function selection module 138 may accept functional near infra-red device data from a frontal near infra-red headset.

Operation 504 depicts accepting functional magnetic resonance imaging data. For example, the at least one device 102, user-health test function selection module 138, and/or brain activity measurement unit 186 may accept functional magnetic resonance imaging data. In one embodiment, user-health test function selection module 138 may accept functional magnetic resonance imaging data in the form of EEG-correlated fMRI data. In some embodiments, a device capable of performing fMRI in real time or near real time may be used as the brain activity measurement unit 186.

Operation 506 depicts accepting at least one of magnetoencephalography data or single photon emission computed tomography data. For example, the at least one device 102, user-health test function selection module 138, and/or brain activity measurement unit 186 may accept at least one of magnetoencephalography data or single photon emission computed tomography data. In one embodiment, user-health test function selection module 138 may accept magnetoencephalography data from a superconducting quantum interference device (SQUID) as the brain activity measurement unit 186. In another embodiment, user-health test function selection module 138 may accept brain image data from a single photon emission computed tomography device, such as a gamma camera.

FIG. 6 illustrates alternative embodiments of the example operational flow 300 of FIG. 3. FIG. 6 illustrates example embodiments where the selecting operation 320 may include at least one additional operation. Additional operations may include operation 600, 602, 604, and/or operation 606.

Operation 600 depicts selecting at least one mental status test function at least partly based on the user brain activity measurement data. For example, device 102 and/or user-health test function selection module 138 may select at least one mental status test function at least partly based on user brain activity measurement data. For example, a user-health test function selection module 138 may select a mental status test function based on EEG data, for example, frontal lobe activation data provided by brain activity measurement unit 186. In general, obtaining brain activity measurement data of a certain type may trigger selection of at least one user-health test function that relates to the brain activity measurement data. For example, brain activity measurement data showing activation of the right prefrontal and right parietal areas may trigger the selection of one or more user-health test functions related to mental status, e.g., an attention test function.

Alternatively, for example, brain activity measurement data showing activation of the lateral temporal lobe may trigger the selection of a corresponding mental status test function relating to language and/or semantic memory. For example, obtaining brain activity measurement data indicating Alzheimer's disease symptoms and/or diagnosis may result in the selection of a corresponding mental status test function, such as a short-term memory test function or a long-term memory test function for the user, for example, to track the severity of the symptoms and/or progression of the disease over time. Selection algorithms may be applied by one of skill in the art according to brain activity measurement data and related known brain functions such as the associations disclosed herein, but not limited to those. An appropriate database may be assembled whereby brain region activity (e.g., prefrontal cortex, temporal lobe, nucleus accumbens) can be matched with an appropriate user-health test function (e.g., attention areas of the brain matched with an attention test function, memory areas of the brain matched with a memory test function, happiness areas of the brain matched with a depression test function). A mental status test function may include, for example, one or more alertness or attention test functions, one or more memory test functions, one more speech test functions, one or more calculation test functions, one or more neglect or construction test functions, and/or one or more sequencing task test functions.

Operation 602 depicts selecting at least one cranial nerve test function at least partly based on the user brain activity measurement data. For example, device 102 and/or user-health test function selection module 138 may select at least one cranial nerve test function at least partly based on the user brain activity measurement data, for example, asymmetric motor cortex activity data provided by brain activity measurement unit 186, possibly indicating an asymmetric cranial nerve lesion, such as Bell's palsy. In some embodiments, cranial nerve lesions in or near the brain may be imaged to detect one or more abnormalities. See Shaikh et al., “Magnetic resonance imaging findings in bilateral Bell's palsy,” J. Neuroimaging, 10(4):223-5 (2000).

Selecting at least one cranial nerve test function may be done based on any obtained user brain activity measurement data, as described above. In general, obtaining user brain activity measurement data of a certain type may trigger selection of at least one user-health test function that relates to the user brain activity measurement data. For example, user brain activity measurement data obtained showing abnormal bilateral enhancement of the proximal intracanalicular segments of VII/VIII nerve complexes may trigger the selection of one or more cranial nerve test functions related to, for example, user face pattern, which is typically affected in Bell's palsy patients.

Alternatively, brain activity data indicating a Bell's palsy symptom and/or diagnosis may result in the selection of a related cranial nerve test function, such as a speech test function. Selection algorithms may be applied by one of skill in the art according to brain activity measurement data and related known user-health test functions, and those disclosed herein.

A cranial nerve test function may include, for example, one or more visual field test functions, one or more eye movement test functions, one more pupil movement test functions, one or more face pattern test functions, one or more hearing test functions, and/or one or more speech and/or voice test functions.

Operation 604 depicts selecting at least one cerebellum test function at least partly based on the user brain activity measurement data. For example, a user-health test function selection module 138 may select at least one cerebellum test function at least partly based on the user brain activity measurement data.

Selecting at least one cerebellum test function may be done based on any obtained brain activity measurement data, as described above. In general, accepting brain activity measurement data of a certain type may trigger selection of at least one user-health test function that relates to the brain activity measurement data. For example, brain activity measurement data showing a cerebellum abnormality may trigger the selection of one or more cerebellum test functions related to user motor skill, gait, and/or coordination, perhaps associated with autism diagnosis and/or symptom analysis.

Alternatively, for example, brain activity measurement data showing a unusual cerebellum activity may trigger the selection of a corresponding motor skill test function. For example, obtaining brain activity measurement data indicating ataxia symptoms or diagnosis may result in the selection of a related cerebellum test function, such as a pointing device manipulation test function and/or an overshoot/past pointing test function. Selection algorithms may be applied by one of skill in the art according to brain activity measurement data and related known user-health test functions, and those disclosed herein. A cerebellum test function may include, for example, one or more body movement test functions and/or one or more motor skill test functions.

Operation 606 depicts selecting at least one of an alertness test function, an attention test function, a memory test function, a speech test function, a calculation test function, a neglect test function, a construction test function, or a task sequencing test function. For example, a user-health test function selection module 138 may select at least one of an alertness test function, an attention test function, a memory test function, a speech test function, a calculation test function, a neglect test function, a construction test function, or a task sequencing test function.

Selecting at least one of an alertness test function, an attention test function, a memory test function, a speech test function, a calculation test function, a neglect test function, a construction test function, or a task sequencing test function may be done based on brain activity measurement data, as described above. In general, obtaining brain activity measurement data of a certain type may trigger selection of at least one user-health test function that relates to the brain activity measurement data. For example, brain activity measurement data showing activity in a memory area of a user's brain may trigger the selection of one or more memory test functions in order to, for example, track memory function over time, or to examine a different aspect of user memory function, such as testing a specific short term memory or long term memory.

Alternatively, for example, brain activity measurement data from a user's medical history may be the basis for the selection of a related user-health test functions. For example, obtaining from a medical records database user brain activity measurement data indicating possible stroke may result in the selection of a related mental status test function, such as a comprehension test function and/or a naming test function to further investigate one or more possible stroke symptoms. Selection algorithms may be applied by one of skill in the art according to brain activity measurement data and related known user-health test functions, and those disclosed herein.

An alertness test function or an attention test function set may include, for example, one or more reaction time test function, one or more spelling test function, and/or one more speech test function.

Alertness or attention user attributes are indicators of a user's mental status. An example of an alertness test function may be a measure of reaction time as one objective manifestation. Examples of attention test functions may include ability to focus on simple tasks, ability to spell the word “world” forward and backward, or reciting a numerical sequence forward and backward as objective manifestations of an alertness problem. An alertness test function and/or user-health test unit 104 may require a user to enter a password backward as a measure of alertness. Alternatively, a user may be prompted to perform an executive function as a predicate to launching an application such as a word processing program. For example, an attention test function could be activated by a user command to open a word processing program, requiring performance of, for example, a spelling task as a preliminary step in launching the word processing program. Also, writing ability may be tested by requiring the user 190 to write their name or write a sentence on a device, perhaps with a stylus on a touchscreen.

Reduced level of alertness or attention can indicate the following possible conditions where an acute reduction in alertness or attention is detected: stroke involving the reticular activating system, stroke involving the bilateral or unilateral thalamus, metabolic abnormalities such as hyper or hypoglycemia, toxic effects due to substance overdose (for example, benzodiazepines, or other toxins such as alcohol). Reduced level of alertness and attention can indicate the following possible conditions where a subacute or chronic reduction in alertness or attention is detected: dementia (caused by, for example, Alzheimer's disease, vascular dementia, Parkinson's disease, Huntingdon's disease, Creutzfeldt-Jakob disease, Pick disease, head injury, infection, normal pressure hydrocephalus, brain tumor, exposure to toxin (for example, lead or other heavy metals), metabolic disorders, hormone disorders, hypoxia, drug reactions, drug overuse, drug abuse, encephalitis (caused by, for example, enteroviruses, herpes viruses, or arboviruses), or mood disorders (for example, bipolar disorder, cyclothymic disorder, depression, depressive disorder NOS (not otherwise specified), dysthymic disorder, postpartum depression, or seasonal affective disorder)).

A reduced level of alertness or attention may indicate certain of the possible conditions discussed above. One skilled in the art can select, establish or determine user-health test functions relating to the one or more types of user-health data indicative of altered alertness or attention associated with a likely condition. Test functions can be chosen by one skilled in the art based on knowledge, direct experience, or using available resources such as websites, textbooks, journal articles, or the like. An example of a relevant website can be found in the online Merck Manual at http://www.merck.com/mmhe/sec06/ch077/ch077c.html#tb077_1. Examples of relevant textbooks include Patten, J. P., “Neurological Differential Diagnosis,” Second Ed., Springer-Verlag, London, 2005; Kasper, Braunwald, Fauci, Hauser, Longo, and Jameson, “Harrison's Principles of Internal Medicine,” 16^{th} Ed., McGraw-Hill, New York, 2005; Greenberg, M. S., “Handbook of Neurosurgery,” to Ed., Thieme, Lakeland, 2006; and Victor, M., and Ropper, A. H., “Adams and Victor's Principles of Neurology,” 7^{th} Ed., McGraw-Hill, New York, 2001.

As another example, a user-health test function selection module 138 may select a memory test function based on brain activity measurement data, for example, fNIR data provided by a brain activity measurement unit 186.

A memory test function may include, for example, one or more word list memory test functions, one or more number memory test functions, and/or one more personal history memory test functions. Another example of a memory test function may include a text or number input device, or user monitoring device prompting a user 190 to, for example, spell, write, speak, or calculate in order to test, for example, short-term memory, long-term memory, or the like.

A user's memory attributes are indicators of a user's mental status. An example of a memory test function may be a measure of a user's short-term ability to recall items presented, for example, in a story, or after a short period of time. Another example of a memory test function may be a measure of a user's long-term memory, for example their ability to remember basic personal information such as birthdays, place of birth, or names of relatives. A memory test function may prompt a user 190 to change and enter a password with a specified frequency during internet browser use. A memory test function involving changes to a password that is required to access an internet server can challenge a user's memory according to a fixed or variable schedule.

Difficulty with recall after about 1 to 5 minutes may indicate damage to the limbic memory structures located in the medial temporal lobes and medial diencephalon of the brain, or damage to the fornix. Dysfunction of these structures characteristically causes anterograde amnesia, meaning difficulty remembering new facts and events occurring after lesion onset. Reduced short-term memory function can also indicate the following conditions: head injury, Alzheimer's disease, Herpes virus infection, seizure, emotional shock or hysteria, alcohol-related brain damage, barbiturate or heroin use, general anaesthetic effects, electroconvulsive therapy effects, stroke, transient ischemic attack (i.e., a “mini-stroke”), complication of brain surgery. Reduced long-term memory function can indicate the following conditions: Alzheimer's disease, alcohol-related brain damage, complication of brain surgery, depressive pseudodementia, adverse drug reactions (e.g., to benzodiazepines, anti-ulcer drugs, analgesics, anti-hypertensives, diabetes drugs, beta-blockers, anti-Parkinson's disease drugs, anti-emetics, anti-psychotics, or certain drug combinations, such as haloperidol and methyldopa combination therapy), multi-infarct dementia, or head injury.

Altered memory attributes may indicate certain of the possible conditions discussed above. One skilled in the art can select, establish or determine user-health test functions relating to the one or more types of user-health data indicative of altered memory associated with a likely condition. Test function sets and test functions can be chosen by one skilled in the art based on knowledge, direct experience, or using available resources such as websites, textbooks, journal articles, or the like. An example of a relevant website can be found in the online Merck Manual at http://www.merck.com/mmhe/sec06/ch077/ch077c.html#tb077_1. Examples of relevant textbooks include Patten, J. P., “Neurological Differential Diagnosis,” Second Ed., Springer-Verlag, London, 2005; Kasper, Braunwald, Fauci, Hauser, Longo, and Jameson, “Harrison's Principles of Internal Medicine,” 16^{th} Ed., McGraw-Hill, New York, 2005; Greenberg, M. S., “Handbook of Neurosurgery,” 6^{th} Ed., Thieme, Lakeland, 2006; and Victor, M., and Ropper, A. H., “Adams and Victor's Principles of Neurology,” 7^{th} Ed., McGraw-Hill, New York, 2001.

A speech test function may include, for example, one or more speech test functions, one more comprehension test functions, one or more naming test functions, and/or one or more reading test functions.

User speech attributes are indicators of a user's mental status. An example of a speech test function may be a measure of a user's fluency or ability to produce spontaneous speech, including phrase length, rate of speech, abundance of spontaneous speech, tonal modulation, or whether paraphasic errors (e.g., inappropriately substituted words or syllables), neologisms (e.g., nonexistent words), or errors in grammar are present. Another example of a speech test function is a program that can measure the number of words spoken by a user during a video conference. The number of words per interaction or per unit time could be measured. A marked decrease in the number of words spoken could indicate a speech problem.

Another example of a voice or speech test function may include tracking of speech or voice data into a device or user monitoring device, such as a telephonic device or a video communication device with sound receiving/transmission capability, for example when a user task requires, for example, speaking, singing, or other vocalization.

Another example of a speech test function may be a measure of a user's comprehension of spoken language, including whether a user 190 can understand simple questions and commands, or grammatical structure. For example, a user-health test function may include a speech or voice analysis module that may ask the user 190 the question “Mike was shot by John. Is John dead?” An inappropriate response may indicate a speech center defect. Alternatively a speech function test may require a user to say a code or phrase and repeat it several times. Speech defects may become apparent if the user has difficulty repeating the code or phrase during, for example, a videoconference setup or while using speech recognition software.

Another example of a speech test function may be a measure of a user's ability to name simple everyday objects (e.g., pen, watch, tie) and also more difficult objects (e.g., fingernail, belt buckle, stethoscope). A speech test function may, for example, require the naming of an object prior to or during the interaction of a user 190 with an application 104, as a time-based or event-based checkpoint. For example, a user 190 may be prompted by a speech test function to say “armadillo” after being shown a picture of an armadillo, prior to or during the user's interaction with, for example, a word processing or email program. A test requiring the naming of parts of objects is often more difficult for users with speech comprehension impairment. Another speech test function may, for example, gauge a user's ability to repeat single words and sentences (e.g., “no if's and's or but's”). A further example of a speech test function measures a user's ability to read single words, a brief written passage, or the front page of the newspaper aloud followed by a test for comprehension.

Difficulty with speech or reading/writing ability may indicate, for example, lesions in the dominant (usually left) frontal lobe, including Broca's area (output area); the left temporal and parietal lobes, including Wernicke's area (input area); subcortical white matter and gray matter structures, including thalamus and caudate nucleus; as well as the non-dominant hemisphere. Typical diagnostic conditions may include, for example, stroke, head trauma, dementia, multiple sclerosis, Parkinson's disease, or Landau-Kleffner syndrome (a rare syndrome of acquired epileptic aphasia).

Altered speech attributes may indicate certain of the possible conditions discussed above. One skilled in the art can select, establish or determine user-health test functions relating to the one or more types of user-health data indicative of altered speech associated with a likely condition. Test function sets and test functions can be chosen by one skilled in the art based on knowledge, direct experience, or using available resources such as websites, textbooks, journal articles, or the like. An example of a relevant website can be found in the online Merck Manual at http://www.merck.com/mmhe/sec06/ch077/ch077c.html#tb077_1. Examples of relevant textbooks include Patten, J. P., “Neurological Differential Diagnosis,” Second Ed., Springer-Verlag, London, 2005; Kasper, Braunwald, Fauci, Hauser, Longo, and Jameson, “Harrison's Principles of Internal Medicine,” 16^{th} Ed., McGraw-Hill, New York, 2005; Greenberg, M. S., “Handbook of Neurosurgery,” 6^{th} Ed., Thieme, Lakeland, 2006; and Victor, M., and Ropper, A. H., “Adams and Victor's Principles of Neurology,” 7^{th} Ed., McGraw-Hill, New York, 2001.

A calculation test function may include, for example, one or more arithmetic test functions involving a user's ability to perform simple math tasks. A user's calculation abilities are indicators of a user's mental status. An example of a calculation test function may be a measure of a user's ability to do simple math such as addition or subtraction, for example. A user 190 may be prompted to solve an arithmetic problem in the context of interacting with application 104, or alternatively, in the context of using the at least one device 102 in between periods of interacting with the application 104. For example, a user may be prompted to calculate the number of items and/or gold pieces collected during a segment of gameplay in the context of playing a game. In this and other contexts, user interaction with a device's operating system or other system functions may also constitute user interaction with an application 104. Difficulty in completing calculation tests may be indicative of stroke (e.g., embolic, thrombotic, or due to vasculitis), dominant parietal lesion, or brain tumor (e.g., glioma or meningioma). When a calculation ability deficiency is found with defects in user ability to distinguish right and left body parts (right-left confusion), ability to name and identify each finger (finger agnosia), and ability to write their name and a sentence (agraphia), Gerstmann syndrome, a lesion in the dominant parietal lobe of the brain, may be present.

Altered calculation ability may indicate certain of the possible conditions discussed above. One skilled in the art can select, establish or determine user-health test functions relating to the one or more types of user-health data indicative of altered calculation ability associated with a likely condition. Test function sets and test functions can be chosen by one skilled in the art based on knowledge, direct experience, or using available resources such as websites, textbooks, journal articles, or the like. An example of a relevant website can be found in the online Merck Manual at http://www.merck.com/mmhe/sec06/ch077/ch077c.html#tb077_1. Examples of relevant textbooks include Patten, J. P., “Neurological Differential Diagnosis,” Second Ed., Springer-Verlag, London, 2005; Kasper, Braunwald, Fauci, Hauser, Longo, and Jameson, “Harrison's Principles of Internal Medicine,” 16^{th} Ed., McGraw-Hill, New York, 2005; Greenberg, M. S. “Handbook of Neurosurgery,” 6^{th} Ed., Thieme, Lakeland, 2006; and Victor, M., and Ropper, A. H., “Adams and Victor's Principles of Neurology,” 7^{th} Ed., McGraw-Hill, New York, 2001.

A neglect test function or a construction test function may include, for example, one or more body movement test functions, one or more pointing device manipulation test functions, and/or one more cognitive test functions such as drawing test functions.

Neglect or construction user attributes are indicators of a user's mental status. Neglect may include a neurological condition involving a deficit in attention to an area of space, often one side of the body or the other. A construction defect may include a deficit in a user's ability to draw complex figures or manipulate blocks or other objects in space as a result of neglect or other visuospatial impairment.

Hemineglect may include an abnormality in attention to one side of the universe that is not due to a primary sensory or motor disturbance. In sensory neglect, users ignore visual, somatosensory, or auditory stimuli on the affected side, despite intact primary sensation. This can often be demonstrated by testing for extinction on double simultaneous stimulation. Thus, a neglect or construction test function set may contain user-health test functions that present a stimulus on one or both sides of a display for a user 190 to click on or otherwise recognize. A user 190 with hemineglect may detect the stimulus on the affected side when presented alone, but when stimuli are presented simultaneously on both sides, only the stimulus on the unaffected side may be detected. In motor neglect, normal strength may be present, however, the user often does not move the affected limb unless attention is strongly directed toward it.

An example of a neglect test function may be a measure of a user's awareness of events occurring on one side of the user or the other. A user could be asked, “Do you see anything on the left side of the screen?” Users with anosognosia (i.e., unawareness of a disability) may be strikingly unaware of severe deficits on the affected side. For example, some people with acute stroke who are completely paralyzed on the left side believe there is nothing wrong and may even be perplexed about why they are in the hospital. Alternatively, a neglect or construction test function set may include a user-health test function that presents a drawing task to a user 190 in the context of an application 104 that involves similar activities. A construction test involves prompting a user to draw complex figures or to manipulate objects in space. Difficulty in completing such a test may be a result of neglect or other visuospatial impairment.

Another neglect test function is a test of a user's ability to acknowledge a series of objects on a display that span a center point on the display. For example, a user may be prompted to click on each of 5 hash marks present in a horizontal line across the midline of a display. If the user has a neglect problem, she may only detect and accordingly click on the hash marks on one side of the display, neglecting the others.

Hemineglect is most common in lesions of the right (nondominant) parietal lobe, causing users to neglect the left side. Left-sided neglect can also occasionally be seen in right frontal lesions, right thalamic or basal ganglia lesions, and, rarely, in lesions of the right midbrain. Hemineglect or difficulty with construction tasks may be indicative of stroke (e.g., embolic, thrombotic, or due to vasculitis), or brain tumor (e.g., glioma or meningioma).

Altered neglect attributes or construction ability may indicate certain of the possible conditions discussed above. One skilled in the art can select, establish or determine user-health test functions relating to the one or more types of user-health data indicative of altered neglect attributes or construction ability associated with a likely condition. Test function sets and test functions can be chosen by one skilled in the art based on knowledge, direct experience, or using available resources such as websites, textbooks, journal articles, or the like. An example of a relevant website can be found in the online Merck Manual at http://www.merck.com/mmhe/sec06/ch077/ch077c.html#tb077_1. Examples of relevant textbooks include Patten, J. P., “Neurological Differential Diagnosis,” Second Ed., Springer-Verlag, London, 2005; Kasper, Braunwald, Fauci, Hauser, Longo, and Jameson, “Harrison's Principles of Internal Medicine,” 16^{th} Ed., McGraw-Hill, New York, 2005; Greenberg, M. S. “Handbook of Neurosurgery,” 6^{th} Ed., Thieme, Lakeland, 2006; and Victor, M., and Ropper, A. H., “Adams and Victor's Principles of Neurology,” 7^{th} Ed., McGraw-Hill, New York, 2001.

A task sequencing test function may include, for example, one or more perseveration test functions such as one or more written alternating sequencing test functions, one or more motor impersistence test functions, or one more behavior control test functions.

A user's task sequencing attributes are indicators of a user's mental status. An example of a task sequencing test function may be a measure of a user's perseveration. For example, at least one device 102 may ask a user to continue drawing a silhouette pattern of alternating triangles and squares (i.e., a written alternating sequencing task) for a time period. In users with perseveration problems, the user may get stuck on one shape and keep drawing triangles. Another common finding is motor impersistence, a form of distractibility in which users only briefly sustain a motor action in response to a command such as “raise your arms” or “look to the right.” Ability to suppress inappropriate behaviors can be tested by the auditory “Go-No-Go” test, in which the user performs a task such as moving an object (e.g., moving a finger) in response to one sound, but must keep the object (e.g., the finger) still in response to two sounds. Alternatively, at least one device 102 may prompt a user to perform a multi-step function in the context of an application 104, for example. For example, a game may prompt a user 190 to enter a character's name, equip an item from an inventory, an click on a certain direction of travel, in that order. Difficulty completing this task may indicate, for example, a frontal lobe defect associated with dementia.

Decreased ability to perform sequencing tasks may be indicative of stroke (e.g., embolic, thrombotic, or due to vasculitis), brain tumor (e.g., glioma or meningioma), or dementia (caused by, for example, Alzheimer's disease, vascular dementia, Parkinson's disease, Huntingdon's disease, Creutzfeldt-Jakob disease, Pick disease, head injury, infection (e.g., meningitis, encephalitis, HIV, or syphilis), normal pressure hydrocephalus, brain tumor, exposure to toxin (for example, lead or other heavy metals), metabolic disorders, hormone disorders, hypoxia (caused by, e.g., emphysema, pneumonia, or congestive heart failure), drug reactions (e.g., anti-cholinergic side effects, drug overuse, drug abuse (e.g., cocaine or heroin).

Altered task sequencing ability may indicate certain of the possible conditions discussed above. One skilled in the art can select, establish or determine user-health test functions relating to the one or more types of user-health data indicative of altered task sequencing ability associated with a likely condition. Test function sets and test functions can be chosen by one skilled in the art based on knowledge, direct experience, or using available resources such as websites, textbooks, journal articles, or the like. An example of a relevant website can be found in the online Merck Manual at http://www.merck.com/mmhe/sec06/ch077/ch077c.html#tb077_1. Examples of relevant textbooks include Patten, J. P., “Neurological Differential Diagnosis,” Second Ed., Springer-Verlag, London, 2005; Kasper, Braunwald, Fauci, Hauser, Longo, and Jameson, “Harrison's Principles of Internal Medicine,” 16^{th} Ed., McGraw-Hill, New York, 2005; Greenberg, M. S. “Handbook of Neurosurgery,” 6^{th} Ed., Thieme, Lakeland, 2006; and Victor, M., and Ropper, A. H., “Adams and Victor's Principles of Neurology,” 7^{th} Ed., McGraw-Hill, New York, 2001.

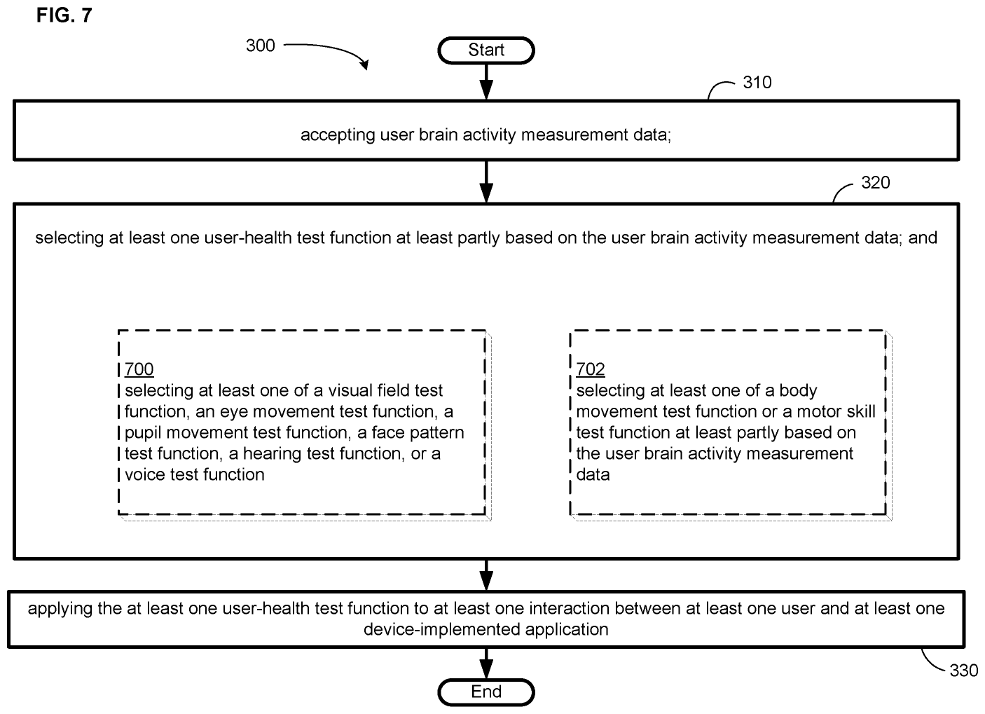

FIG. 7 illustrates alternative embodiments of the example operational flow 300 of FIG. 3. FIG. 7 illustrates example embodiments where the selecting operation 320 may include at least one additional operation. Additional operations may include operation 700 and/or operation 702.

Operation 700 depicts selecting at least one of a visual field test function, an eye movement test function, a pupil movement test function, a face pattern test function, a hearing test function, or a voice test function. For example, device 102 and/or user-health test function selection module 138 may select at least one of a visual field test function, an eye movement test function, a pupil movement test function, a face pattern test function, a hearing test function, or a voice test function. In one embodiment, user-health test function selection module 138 may select a visual field test function based on brain activity measurement data, for example, visual cortex activation data provided by brain activity measurement unit 186.

Selecting at least one of a visual field test function, an eye movement test function, a pupil movement test function, a face pattern test function, a hearing test function, or a voice test function may be done based on obtained brain activity measurement data, as described above. In general, obtaining brain activity measurement data of a certain type may trigger selection of at least one user-health test function that relates to the brain activity measurement data. For example, brain activity measurement data showing altered activity in the visual cortex may trigger the selection of one or more visual field test functions in order to track eye movement and/or visual field over time, or to examine a different aspect of user vision (e.g., visual acuity).

Alternatively, for example, brain activity measurement data from a user's medical history may trigger the selection of related user-health test functions. For example, obtaining from a medical records database information indicating normal brain activity in the speech areas of a patient having speech difficulty may result in the selection of a related cranial nerve test function, such as a voice test function to measure vagus nerve damage, e.g., via vocal chord function. Selection algorithms may be applied by one of skill in the art according to brain activity measurement data and related known user-health test functions, and those disclosed herein.

A visual field test function may include, for example, one or more visual field test functions, one or more pointing device manipulation test functions, and/or one more reading test functions.

Visual field user attributes are indicators of a user's ability to see directly ahead and peripherally. An example of a visual field test function may be a measure of a user's gross visual acuity, for example using a Snellen eye chart or visual equivalent on a display. Alternatively, a campimeter may be used to conduct a visual field test. A device 102 and/or user-health test function unit 140 may contain a user-health test function set 196 including a user-health test function that may prompt a user 190 to activate a portion of a display when the user 190 can detect an object entering their field of view from a peripheral location relative to a fixed point of focus, either with both eyes or with one eye covered at a time. Such testing could be done in the context of, for example, new email alerts that require clicking and that appear in various locations on a display. Based upon the location of decreased visual field, the defect can be localized, for example in a quadrant system. A pre-chiasmatic lesion results in ipsilateral eye blindness. A chiasmatic lesion can result in bi-temporal hemianopsia (i.e., tunnel vision). Post-chiasmatic lesions proximal to the geniculate ganglion can result in left or right homonymous hemianopsia. Lesions distal to the geniculate ganglion can result in upper or lower homonymous quadrantanopsia.

Visual field defects may indicate optic nerve conditions such as pre-chiasmatic lesions, which include fractures of the sphenoid bone (e.g., transecting the optic nerve), retinal tumors, or masses compressing the optic nerve. Such conditions may result in unilateral blindness and unilaterally unreactive pupil (although the pupil may react to light applied to the contralateral eye). Bi-temporal hemianopsia can be caused by glaucoma, pituitary adenoma, craniopharyngioma or saccular Berry aneurysm at the optic chiasm. Post-chiasmatic lesions are associated with homonymous hemianopsia or quadrantanopsia depending on the location of the lesion.

Altered visual field may indicate certain of the possible conditions discussed above. One skilled in the art can select, establish or determine user-health test functions relating to the one or more types of user-health data indicative of altered visual field associated with a likely condition. Test function sets and test functions can be chosen by one skilled in the art based on knowledge, direct experience, or using available resources such as websites, textbooks, journal articles, or the like. An example of a relevant website can be found in the online Merck Manual at http://www.merck.com/mmhe/sec06/ch077/ch077c.html#tb077_1. Examples of relevant textbooks include Patten, J. P., “Neurological Differential Diagnosis,” Second Ed., Springer-Verlag, London, 2005; Kasper, Braunwald, Fauci, Hauser, Longo, and Jameson, “Harrison's Principles of Internal Medicine,” 16^{th} Ed., McGraw-Hill, New York, 2005; Greenberg, M. S., “Handbook of Neurosurgery,” 6th Ed., Thieme, Lakeland, 2006; and Victor, M., and Ropper, A. H., “Adams and Victor's Principles of Neurology,” 7^{th} Ed., McGraw-Hill, New York, 2001.

An eye movement test function or a pupil movement test function may include, for example, one or more eye movement test functions, one more pupil movement test functions, and/or one or more pointing device manipulation test functions.

An example of an eye movement test function may be a measurement of a user's ability to follow a target on a display with her eyes throughout a 360° range. Such testing may be done in the context of a user playing a game or participating in a videoconference. In such examples, user-health data 116 may be obtained through a camera in place as a user monitoring device 182 that can monitor the eye movements of the user during interaction with the application 104.

Another example of an eye movement test function may include eye tracking data from a user monitoring device, such as a video communication device, for example, when a user task requires tracking objects on a display, reading, or during resting states between activities in an application. A further example includes pupil movement tracking data from the user 190 at rest or during an activity required by an application or user-health test function.

Testing of the trochlear nerve or the abducens nerve for damage may involve measurement of extraocular movements. The trochlear nerve performs intorsion, depression, and abduction of the eye. A trochlear nerve lesion may present as extorsion of the ipsilateral eye and worsened diplopia when looking down. Damage to the abducens nerve may result in a decreased ability to abduct the eye.

Abnormalities in eye movement may indicate fracture of the sphenoid wing, intracranial hemorrhage, neoplasm, or aneurysm. Such insults may present as extorsion of the ipsilateral eye. Individuals with this condition complain of worsened diplopia with attempted downgaze, but improved diplopia with head tilted to the contralateral side. Injury to the abducens nerve may be caused by aneurysm, a mass in the cavernous sinus, or a fracture of the skull base. Such insults may result in extraocular palsy defined by medial deviation of the ipsilateral eye. Users with this condition may present with diplopia that improves when the contralateral eye is abducted.

Nystagmus is a rapid involuntary rhythmic eye movement, with the eyes moving quickly in one direction (quick phase), and then slowly in the other direction (slow phase). The direction of nystagmus is defined by the direction of its quick phase (e.g., right nystagmus is due to a right-moving quick phase). Nystagmus may occur in the vertical or horizontal directions, or in a semicircular movement. Terminology includes downbeat nystagmus, upbeat nystagmus, seesaw nystagmus, periodic alternating nystagmus, and pendular nystagmus. There are other similar alterations in periodic eye movements (saccadic oscillations) such as opsoclonus or ocular flutter. One can think of nystagmus as the combination of a slow adjusting eye movement (slow phase) as would be seen with the vestibulo-ocular reflex, followed by a quick saccade (quick phase) when the eye has reached the limit of its rotation.

In medicine, the clinical importance of nystagmus is that it indicates that the user's spatial sensory system perceives rotation and is rotating the eyes to adjust. Thus it depends on the coordination of activities between two major physiological systems: the vision and the vestibular apparatus (which controls posture and balance). This may be physiological (i.e., normal) or pathological.

Vestibular nystagmus may be central or peripheral. Important differentiating features between central and peripheral nystagmus include the following: peripheral nystagmus is unidirectional with the fast phase opposite the lesion; central nystagmus may be unidirectional or bidirectional; purely vertical or torsional nystagmus suggests a central location; central vestibular nystagmus is not dampened or inhibited by visual fixation; tinnitus or deafness often is present in peripheral vestibular nystagmus, but it usually is absent in central vestibular nystagmus. According to Alexander's law, the nystagmus associated with peripheral lesions becomes more pronounced with gaze toward the side of the fast-beating component; with central nystagmus, the direction of the fast component is directed toward the side of gaze (e.g., left-beating in left gaze, right-beating in right gaze, and up-beating in upgaze).

Downbeat nystagmus is defined as nystagmus with the fast phase beating in a downward direction. The nystagmus usually is of maximal intensity when the eyes are deviated temporally and slightly inferiorly. With the eyes in this position, the nystagmus is directed obliquely downward. In most users, removal of fixation (e.g., by Frenzel goggles) does not influence slow phase velocity to a considerable extent, however, the frequency of saccades may diminish.

The presence of downbeat nystagmus is highly suggestive of disorders of the cranio-cervical junction (e.g., Arnold-Chiari malformation). This condition also may occur with bilateral lesions of the cerebellar flocculus and bilateral lesions of the medial longitudinal fasciculus, which carries optokinetic input from the posterior semicircular canals to the third nerve nuclei. It may also occur when the tone within pathways from the anterior semicircular canals is relatively higher than the tone within the posterior semicircular canals. Under such circumstances, the relatively unopposed neural activity from the anterior semicircular canals causes a slow upward pursuit movement of the eyes with a fast, corrective downward saccade. Additional causes include demyelination (e.g., as a result of multiple sclerosis), microvascular disease with vertebrobasilar insufficiency, brain stem encephalitis, tumors at the foramen magnum (e.g., meningioma, or cerebellar hemangioma), trauma, drugs (e.g., alcohol, lithium, or anti-seizure medications), nutritional imbalances (e.g., Wernicke encephalopathy, parenteral feeding, magnesium deficiency), or heat stroke.

Upbeat nystagmus is defined as nystagmus with the fast phase beating in an upward direction. Daroff and Troost described two distinct types. The first type consists of a large amplitude nystagmus that increases in intensity with upward gaze. This type is suggestive of a lesion of the anterior vermis of the cerebellum. The second type consists of a small amplitude nystagmus that decreases in intensity with upward gaze and increases in intensity with downward gaze. This type is suggestive of lesions of the medulla, including the perihypoglossal nuclei, the adjacent medial vestibular nucleus, and the nucleus intercalatus (structures important in gaze-holding). Upbeat nystagmus may also be an indication of benign paroxysmal positional vertigo.

Torsional (rotary) nystagmus refers to a rotary movement of the globe about its anteroposterior axis. Torsional nystagmus is accentuated on lateral gaze. Most nystagmus resulting from dysfunction of the vestibular system has a torsional component superimposed on a horizontal or vertical nystagmus. This condition occurs with lesions of the anterior and posterior semicircular canals on the same side (e.g., lateral medullary syndrome or Wallenberg syndrome). Lesions of the lateral medulla may produce a torsional nystagmus with the fast phase directed away from the side of the lesion. This type of nystagmus can be accentuated by otolithic stimulation by placing the user on their side with the intact side down (e.g., if the lesion is on the left, the nystagmus is accentuated when the user is placed on his right side).

This condition may occur when the tone within the pathways of the posterior semicircular canals is relatively higher than the tone within the anterior semicircular canals, and it can occur from lesions of the ventral tegmental tract or the brachium conjunctivum, which carry optokinetic input from the anterior semicircular canals to the third nerve nuclei.

Pendular nystagmus is a multivectorial nystagmus (i.e., horizontal, vertical, circular, and elliptical) with an equal velocity in each direction that may reflect brain stem or cerebellar dysfunction. Often, there is marked asymmetry and dissociation between the eyes. The amplitude of the nystagmus may vary in different positions of gaze. Causes of pendular nystagmus may include demyelinating disease, monocular or binocular visual deprivation, oculapalatal myoclonus, internuclear ophthalmoplegia, or brain stem or cerebellar dysfunction.

Horizontal nystagmus is a well-recognized finding in patients with a unilateral disease of the cerebral hemispheres, especially with large, posterior lesions. It often is of low amplitude. Such patients show a constant velocity drift of the eyes toward the intact hemisphere with fast saccade directed toward the side of the lesion.

Seesaw nystagmus is a pendular oscillation that consists of elevation and intorsion of one eye and depression and extorsion of the fellow eye that alternates every half cycle. This striking and unusual form of nystagmus may be seen in patients with chiasmal lesions, suggesting loss of the crossed visual inputs from the decussating fibers of the optic nerve at the level of the chiasm as the cause or lesions in the rostral midbrain. This type of nystagmus is not affected by otolithic stimulation. Seesaw nystagmus may also be caused by parasellar lesions or visual loss secondary to retinitis pigmentosa.

Gaze-evoked nystagmus is produced by the attempted maintenance of an extreme eye position. It is the most common form of nystagmus. Gaze-evoked nystagmus is due to a deficient eye position signal in the neural integrator network. Thus, the eyes cannot be maintained at an eccentric orbital position and are pulled back toward primary position by the elastic forces of the orbital fascia. Then, corrective saccade moves the eyes back toward the eccentric position in the orbit.

Gaze-evoked nystagmus may be caused by structural lesions that involve the neural integrator network, which is dispersed between the vestibulocerebellum, the medulla (e.g., the region of the nucleus prepositus hypoglossi and adjacent medial vestibular nucleus “NPH/MVN”), and the interstitial nucleus of Cajal (“INC”). Patients recovering from a gaze palsy go through a period where they are able to gaze in the direction of the previous palsy, but they are unable to sustain gaze in that direction; therefore, the eyes drift slowly back toward primary position followed by a corrective saccade. When this is repeated, a gaze-evoked or gaze-paretic nystagmus results.

Gaze-evoked nystagmus often is encountered in healthy users; in which case, it is called end-point nystagmus. End-point nystagmus usually can be differentiated from gaze-evoked nystagmus caused by disease, in that the former has lower intensity and, more importantly, is not associated with other ocular motor abnormalities. Gaze-evoked nystagmus also may be caused by alcohol or drugs including anti-convulsants (e.g., phenobarbital, phenytoin, or carbamazepine) at therapeutic dosages.

Spasmus nutans is a rare condition with the clinical triad of nystagmus, head nodding, and torticollis. Onset is from age 3-15 months with disappearance by 3 or 4 years. Rarely, it may be present to age 5-6 years. The nystagmus typically consists of small-amplitude, high frequency oscillations and usually is bilateral, but it can be monocular, asymmetric, and variable in different positions of gaze. Spasmus nutans occurs in otherwise healthy children. Chiasmal, suprachiasmal, or third ventricle gliomas may cause a condition that mimics spasmus nutans.

Periodic alternating nystagmus is a conjugate, horizontal jerk nystagmus with the fast phase beating in one direction for a period of approximately 1-2 minutes. The nystagmus has an intervening neutral phase lasting 10-20 seconds; the nystagmus begins to beat in the opposite direction for 1-2 minutes; then the process repeats itself. The mechanism may be disruption of the vestibulo-ocular tracts at the pontomedullary junction. Causes of periodic alternating nystagmus may include Arnold-Chiari malformation, demyelinating disease, spinocerebellar degeneration, lesions of the vestibular nuclei, head trauma, encephalitis, syphilis, posterior fossa tumors, or binocular visual deprivation (e.g., ocular media opacities).

Abducting nystagmus of internuclear ophthalmoplegia (“INO”) is nystagmus in the abducting eye contralateral to a medial longitudinal fasciculus (“MLF”) lesion.

An example of a pupil movement test function may be a measure of a user's pupils when exposed to light or objects at various distances. A pupillary movement test may assess the size and symmetry of a user's pupils before and after a stimulus, such as light or focal point. Anisocoria (i.e., unequal pupils) of up to 0.5 mm is fairly common, and is benign provided pupillary reaction to light is normal. Pupillary reflex can be tested in a darkened room by shining light in one pupil and observing any constriction of the ipsilateral pupil (direct reflex) or the contralateral pupil (contralateral reflex). If abnormality is found with light reaction, pupillary accommodation can be tested by having the user focus on an object at a distance, then focus on the object at about 10 cm from the nose. Pupils should converge and constrict at close focus.

Pupillary abnormalities may be a result of either optic nerve or oculomotor nerve lesions. An optic nerve lesion (e.g., blind eye) will not react to direct light and will not elicit a consensual pupillary constriction, but will constrict if light is shown in the opposite eye. A Homer's syndrome lesion (sympathetic chain lesion) can also present as a pupillary abnormality. In Homer's syndrome, the affected pupil is smaller but constricts to both light and near vision and may be associated with ptosis and anhydrosis. In an oculomotor nerve lesion, the affected pupil is fixed and dilated and may be associated with ptosis and lateral deviation (due to unopposed action of the abducens nerve). Small pupils that do not react to light but do constrict with near vision (i.e., accommodate but do not react to light) can be seen in central nervous system syphilis (“Argyll Robertson pupil”).

Pupillary reflex deficiencies may indicate damage to the oculomotor nerve in basilar skull fracture or uncal herniation as a result of increased intracranial pressure. Masses or tumors in the cavernous sinus, syphilis, or aneurysm may also lead to compression of the oculomotor nerve. Injury to the oculomotor nerve may result in ptosis, inferolateral displacement of the ipsilateral eye (which can present as diplopia or strabismus), or mydriasis.

Altered eye movement ability or pupil movement ability may indicate certain of the possible conditions discussed above. One skilled in the art can select, establish or determine user-health test functions relating to the one or more types of user-health data indicative of altered eye movement ability or pupil movement ability associated with a likely condition. Test function sets and test functions can be chosen by one skilled in the art based on knowledge, direct experience, or using available resources such as websites, textbooks, journal articles, or the like. An example of a relevant web site can be found in the online Merck Manual at http://www.merck.cam/mmhe/sec06/ch077/ch077c.html#tb077_1. Examples of relevant textbooks include Patten, J. P., “Neurological Differential Diagnosis,” Second Ed., Springer-Verlag, London, 2005; Kasper, Braunwald, Fauci, Hauser, Longo, and Jameson, “Harrison's Principles of Internal Medicine,” 16^{th} Ed., McGraw-Hill, New York, 2005; Greenberg, M. S. “Handbook of Neurosurgery,” 6^{th} Ed., Thieme, Lakeland, 2006; and Victor, M., and Ropper, A. H., “Adams and Victor's Principles of Neurology,” 7^{th} Ed., McGraw-Hill, New York, 2001.

A face pattern test function may include, for example, one or more face movement test functions involving a user's ability to move the muscles of the face. An example of a face pattern test function may be a comparison of a user's face while at rest, specifically looking for nasolabial fold flattening or drooping of the corner of the mouth, with the user's face while moving certain facial features. The user may be asked to raise her eyebrows, wrinkle her forehead, show her teeth, puff out her cheeks, or close her eyes tight. Such testing may done via facial pattern recognition software used in conjunction with, for example, a videoconferencing application. Any weakness or asymmetry may indicate a lesion in the facial nerve. In general, a peripheral lesion of the facial nerve may affect the upper and lower face while a central lesion may only affect the lower face.

Abnormalities in facial expression or pattern may indicate a petrous fracture. Peripheral facial nerve injury may also be due to compression, tumor, or aneurysm. Bell's Palsy is thought to be caused by idiopathic inflammation of the facial nerve within the facial canal. A peripheral facial nerve lesion involves muscles of both the upper and lower face and can involve loss of taste sensation from the anterior ⅔ of the tongue (via the chorda tympani). A central facial nerve palsy due to tumor or hemorrhage results in sparing of upper and frontal orbicularis occuli due to crossed innervation. Spared ability to raise eyebrows and wrinkle the forehead helps differentiate a peripheral palsy from a central process. This also may indicate stroke or multiple sclerosis.

Altered face pattern may indicate certain of the possible conditions discussed above. One skilled in the art can select, establish or determine user-health test functions relating to the one or more types of user-health data and/or brain activity measurement data indicative of altered face pattern associated with a likely condition. Test function sets and test functions can be chosen by one skilled in the art based on knowledge, direct experience, or using available resources such as websites, textbooks, journal articles, or the like. An example of a relevant website can be found in the online Merck Manual at http://www.merck.com/mmhe/sec06/ch077/ch077c.html#tb077_1. Examples of relevant textbooks include Patten, J. P., “Neurological Differential Diagnosis,” Second Ed., Springer-Verlag, London, 2005; Kasper, Braunwald, Fauci, Hauser, Longo, and Jameson, “Harrison's Principles of Internal Medicine,” 16^{th} Ed., McGraw-Hill, New York, 2005; Greenberg, M. S., “Handbook of Neurosurgery,” 6^{th} Ed., Thieme, Lakeland, 2006; and Victor, M., and Ropper, A. H., “Adams and Victor's Principles of Neurology,” 7^{th} Ed., McGraw-Hill, New York, 2001.

A hearing test function may include, for example, one or more conversation hearing test functions such as one or more tests of a user's ability to detect conversation, for example in a teleconference or videoconference scenario, one or more music detection test functions, or one more device sound effect test functions, for example in a game scenario.

An example of a hearing test function may be a gross hearing assessment of a user's ability to hear sounds. This can be done by simply presenting sounds to the user or determining if the user can hear sounds presented to each of the ears. For example, at least one device 102 may vary volume settings or sound frequency on a user's device 102 or within an application 104 over time to test user hearing. For example, a mobile phone device or other communication device may carry out various hearing test functions.

Petrous fractures that involve the vestibulocochlear nerve may result in hearing loss, vertigo, or nystagmus (frequently positional) immediately after the injury. Severe middle ear infection can cause similar symptoms but have a more gradual onset. Acoustic neuroma is associated with gradual ipsilateral hearing loss. Due to the close proximity of the vestibulocochlear nerve with the facial nerve, acoustic neuromas often present with involvement of the facial nerve. Neurofibromatosis type II is associated with bilateral acoustic neuromas. Vertigo may be associated with anything that compresses the vestibulocochlear nerve including vascular abnormalities, inflammation, or neoplasm.

Altered hearing ability may indicate certain of the possible conditions discussed above. One skilled in the art can select, establish or determine user-health test functions relating to the one or more types of user-health data indicative of altered hearing ability associated with a likely condition. Test function sets and test functions can be chosen by one skilled in the art based on knowledge, direct experience, or using available resources such as websites, textbooks, journal articles, or the like. An example of a relevant website can be found in the online Merck Manual at http://www.merck.com/mmhe/sec06/ch077/ch077c.html#tb077_1. Examples of relevant textbooks include Patten, J. P., “Neurological Differential Diagnosis,” Second Ed., Springer-Verlag, London, 2005; Kasper, Braunwald, Fauci, Hauser, Longo, and Jameson, “Harrison's Principles of Internal Medicine,” 16^{th} Ed., McGraw-Hill, New York, 2005; Greenberg, M. S., “Handbook of Neurosurgery,” 6^{th} Ed., Thieme, Lakeland, 2006; and Victor, M., and Ropper, A. H., “Adams and Victor's Principles of Neurology,” 7^{th} Ed., McGraw-Hill, New York, 2001.

A voice test function may include, for example, one or more voice test functions. An example of a voice test function may be a measure of symmetrical elevation of the palate when the user says “aah,” or a test of the gag reflex. In an ipsilateral lesion of the vagus nerve, the uvula deviates towards the affected side. As a result of its innervation (through the recurrent laryngeal nerve) to the vocal cords, hoarseness may develop as a symptom of vagus nerve injury. A voice test function and/or user-health test unit 104 may monitor user voice frequency or volume data during, for example, gaming, videoconferencing, speech recognition software use, or mobile phone use. Injury to the recurrent laryngeal nerve can occur with lesions in the neck or apical chest. The most common lesions are tumors in the neck or apical chest. Cancers may include lung cancer, esophageal cancer, or squamous cell cancer.

Other voice test functions may involve first observing the tongue (while in floor of mouth) for fasciculations. If present, fasciculations may indicate peripheral hypoglossal nerve dysfunction. Next, the user may be prompted to protrude the tongue and move it in all directions. When protruded, the tongue will deviate toward the side of a lesion (as the unaffected muscles push the tongue more than the weaker side). Gross symptoms of pathology may result in garbled sound in speech (as if there were marbles in the user's mouth). Damage to the hypoglossal nerve affecting voice/speech may indicate neoplasm, aneurysm, or other external compression, and may result in protrusion of the tongue away from side of the lesion for an upper motor neuron process and toward the side of the lesion for a lower motor neuron process. Accordingly, a voice test function and/or user-health test unit 104 may assess a user's ability to make simple sounds or to say words, for example, consistently with an established voice pattern for the user.

Altered voice may indicate certain of the possible conditions discussed above. One skilled in the art can select, establish or determine user-health test functions relating to the one or more types of user-health data indicative of altered voice associated with a likely condition. Test function sets and test functions can be chosen by one skilled in the art based on knowledge, direct experience, or using available resources such as websites, textbooks, journal articles, or the like. An example of a relevant website can be found in the online Merck Manual at http://www.merck.com/mmhe/sec06/ch077/ch077c.html#tb077_1. Examples of relevant textbooks include Patten, J. P., “Neurological Differential Diagnosis,” Second Ed., Springer-Verlag, London, 2005; Kasper, Braunwald, Fauci, Hauser, Longo, and Jameson, “Harrison's Principles of Internal Medicine,” 16^{th} Ed., McGraw-Hill, New York, 2005; Greenberg, M. S. “Handbook of Neurosurgery,” 6^{th} Ed., Thieme, Lakeland, 2006; and Victor, M., and Ropper, A. H., “Adams and Victor's Principles of Neurology,” 7^{th} Ed., McGraw-Hill, New York, 2001.

Operation 702 depicts selecting at least one of a body movement test function or a motor skill test function at least partly based on the user brain activity measurement data. For example, device 102 and/or user-health test function selection module 138 may select at least one of a body movement test function or a motor skill test function at least partly based on the user brain activity measurement data. In one embodiment, user-health test function selection module 138 may select a body movement test function or a motor skill test function based on user brain activity measurement data, for example, cerebellum test function output data provided by brain activity measurement unit 186.

An example of a body movement test function may include prompting a user 190 to activate or click a specific area on a display to test, for example, arm movement, hand movement, or other body movement or motor skill function. Another example is visual tracking of a user's body, for example during a videoconference, wherein changes in facial movement, limb movement, or other body movements are detectable. A further example is testing a user's ability to move while using a game controller containing an accelerometer, for example, the Wii remote that is used for transmitting user movement data to a computing device.

Another example of a body movement test function may be first observing the user for atrophy or fasciculation in the trapezius muscles, shoulder drooping, or displacement of the scapula. A body movement test function may then prompt the user to turn the head and shrug shoulders against resistance. Weakness in turning the head in one direction may indicate a problem in the contralateral spinal accessory nerve, while weakness in shoulder shrug may indicate an ipsilateral spinal accessory nerve lesion. Ipsilateral paralysis of the sternocleidomastoid and trapezius muscles due to neoplasm, aneurysm, or radical neck surgery also may indicate damage to the spinal accessory nerve. A body movement test function may perform gait analysis, for example, in the context of a security system surveillance application involving video monitoring of the user.

Cerebellar disorders can disrupt body coordination or gait while leaving other motor functions relatively intact. The term ataxia is often used to describe the abnormal movements seen in coordination disorders. In ataxia, there are medium- to large-amplitude involuntary movements with an irregular oscillatory quality superimposed on and interfering with the normal smooth trajectory of movement. Overshoot is also commonly seen as part of ataxic movements and is sometimes referred to as “past pointing” when target-oriented movements are being discussed. Another feature of coordination disorders is dysdiadochokinesia (i.e., abnormal alternating movements). Cerebellar lesions can cause different kinds of coordination problems depending on their location. One important distinction is between truncal ataxia and appendicular ataxia. Appendicular ataxia affects movements of the extremities and is usually caused by lesions of the cerebellar hemispheres and associated pathways. Truncal ataxia affects the proximal musculature, especially that involved in gait stability, and is caused by midline damage to the cerebellar vermis and associated pathways.

A body movement user-health test function may also include a user-health test function of fine movements of the hands and feet. Rapid alternating movements, such as wiping one palm alternately with the palm and dorsum of the other hand, may be tested as well. A common test of coordination is the finger-nose-finger test, in which the user is asked to alternately touch their nose and an examiner's finger as quickly as possible. Ataxia may be revealed if the examiner's finger is held at the extreme of the user's reach, and if the examiner's finger is occasionally moved suddenly to a different location. Overshoot may be measured by having the user raise both arms suddenly from their lap to a specified level in the air. In addition, pressure can be applied to the user's outstretched arms and then suddenly released. Alternatively, testing of fine movements of the hands may be tested by measuring a user's ability to make fine movements of a cursor on a display. To test the accuracy of movements in a way that requires very little strength, a user can be prompted to repeatedly touch a line drawn on the crease of the user's thumb with the tip of their forefinger; alternatively, a user may be prompted to repeatedly touch an object on a touchscreen display.

Normal performance of motor tasks depends on the integrated functioning of multiple sensory and motor subsystems. These include position sense pathways, lower motor neurons, upper motor neurons, the basal ganglia, and the cerebellum. Thus, in order to convincingly demonstrate that abnormalities are due to a cerebellar lesion, one should first test for normal joint position sense, strength, and reflexes and confirm the absence of involuntary movements caused by basal ganglia lesions. As discussed above, appendicular ataxia is usually caused by lesions of the cerebellar hemispheres and associated pathways, while truncal ataxia is often caused by damage to the midline cerebellar vermis and associated pathways.

Another body movement test is the Romberg test, which may indicate a problem in the vestibular or proprioception system. A user is asked to stand with feet together (touching each other). Then the user is prompted to close their eyes. If a problem is present, the user may begin to sway or fall. With the eyes open, three sensory systems provide input to the cerebellum to maintain truncal stability. These are vision, proprioception, and vestibular sense. If there is a mild lesion in the vestibular or proprioception systems, the user is usually able to compensate with the eyes open. When the user closes their eyes, however, visual input is removed and instability can be brought out. If there is a more severe proprioceptive or vestibular lesion, or if there is a midline cerebellar lesion causing truncal instability, the user will be unable to maintain this position even with their eyes open.

A motor skill test function may include, for example, one or more deliberate body movement test functions such as one or more tests of a user's ability to move an object, including objects on a display, e.g., a cursor.

An example of a motor skill test function may be a measure of a user's ability to perform a physical task. A motor skill test function may measure, for example, a user's ability to traverse a path on a display in straight line with a pointing device, to type a certain sequence of characters without error, or to type a certain number of characters without repetition. For example, a wobbling cursor on a display may indicate ataxia in the user, or a wobbling cursor while the user is asked to maintain the cursor on a fixed point on a display may indicate early Parkinson's disease symptoms. Alternatively, a user may be prompted to switch tasks, for example, to alternately type some characters using a keyboard and click on some target with a mouse. If a user has a motor skill deficiency, she may have difficulty stopping one task and starting the other task.

In clinical practice, characterization of tremor is important for etiologic consideration and treatment. Common types of tremor include resting tremor, postural tremor, action or kinetic tremor, task-specific tremor, or intention or terminal tremor. Resting tremor occurs when a body part is at complete rest against gravity. Tremor amplitude tends to decrease with voluntary activity. Causes of resting tremor may include Parkinson's disease, Parkinson-plus syndromes (e.g., multiple system atrophy, progressive supranuclear palsy, or corticobasal degeneration), Wilson's disease, drug-induced Parkinsonism (e.g., neuroleptics, Reglan, or phenthiazines), or long-standing essential tremor.

Postural tremor occurs during maintenance of a position against gravity and increases with action. Action or kinetic tremor occurs during voluntary movement. Examples of postural and action tremors may include essential tremor (primarily postural), metabolic disorders (e.g., thyrotoxicosis, pheochromocytoma, or hypoglycemia), drug-induced parkinsonism (e.g., lithium, amiodarone, or beta-adrenergic agonists), toxins (e.g., alcohol withdrawal, heavy metals), neuropathic tremor (e.g., neuropathy).

Task-specific tremor emerges during specific activity. An example of this type is primary writing tremor. Intention or terminal tremor manifests as a marked increase in tremor amplitude during a terminal portion of targeted movement. Examples of intention tremor include cerebellar tremor and multiple sclerosis tremor.

Altered body movement or motor skill may indicate certain of the possible conditions discussed above. One skilled in the art can select, establish or determine user-health test functions relating to the one or more types of user-health data indicative of altered body movement or motor skill associated with a likely condition. Test function sets and test functions can be chosen by one skilled in the art based on knowledge, direct experience, or using available resources such as websites, textbooks, journal articles, or the like. An example of a relevant website can be found in the online Merck Manual at http://www.merck.com/mmhe/sec06/ch077/ch077c.html#tb077_1. Examples of relevant textbooks include Patten, J. P., “Neurological Differential Diagnosis,” Second Ed., Springer-Verlag, London, 2005; Kasper, Braunwald, Fauci, Hauser, Longo, and Jameson, “Harrison's Principles of Internal Medicine,” 16^{th} Ed., McGraw-Hill, New York, 2005; Greenberg, M. S. “Handbook of Neurosurgery,” 6^{th} Ed., Thieme, Lakeland, 2006; and Victor, M., and Ropper, A. H., “Adams and Victor's Principles of Neurology,” 7^{th} Ed., McGraw-Hill, New York, 2001.

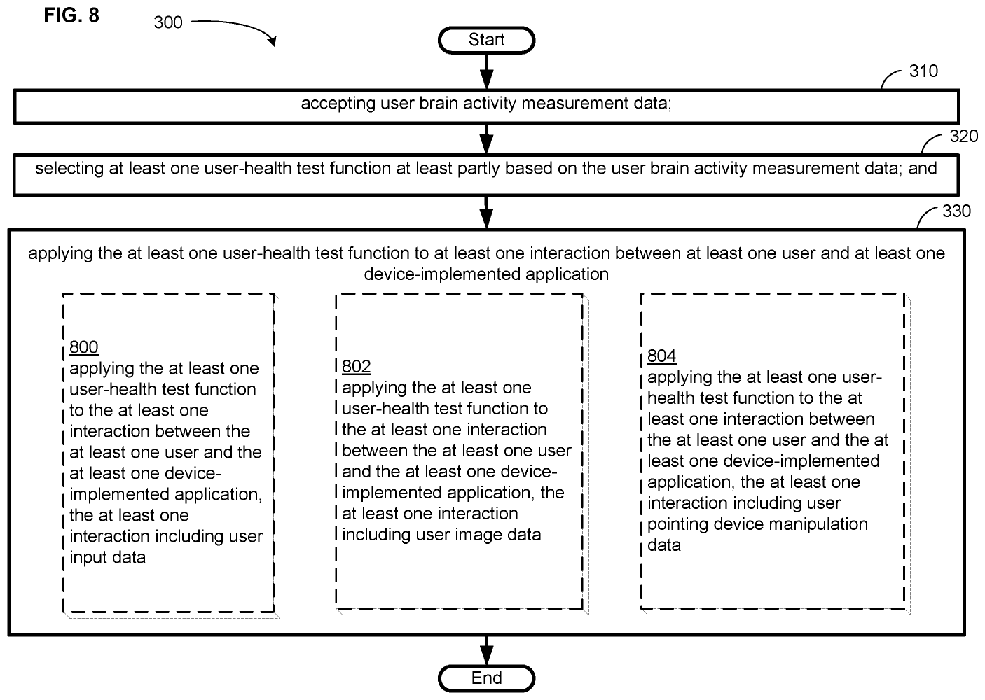

FIG. 8 illustrates alternative embodiments of the example operational flow 300 of FIG. 3. FIG. 8 illustrates example embodiments where the applying operation 330 may include at least one additional operation. Additional operations may include operation 800, 802 and/or operation 804.

Operation 800 depicts applying the at least one user-health test function to the at least one interaction between the at least one user and the at least one device-implemented application, the at least one interaction including user input data. For example, device 102 and/or user-health test function unit 140 may apply the at least one user-health test function to the at least one interaction between the at least one user and the at least one device-implemented application, the at least one interaction including user input data. In one embodiment, the at least one device 102, user-health test function unit 140 and/or user-health test function selection module 138 can apply at least one attention test function to an interaction between a user 190 and an interactive application on a web browser. The attention test function may act in conjunction with the interactive application on the web browser to prompt the user to enter keystroke data to complete the attention test, for example spelling a word forward and backwards, or typing a block of text with a certain level of fidelity. Other examples of user input data include activating a touchscreen by tapping or other means, and user voice input. Other examples of appropriate contexts for user input data may include memory test functions, task sequencing functions, and/or motor skill test functions. In another embodiment, the device 102 and/or user-health test function unit 140 may apply a user-health test function in response to, for example, a user-health test function selection module 138 selecting the user-health test function at least partly based on a user's brain activity measurement data.

Operation 802 depicts applying the at least one user-health test function to the at least one interaction between the at least one user and the at least one device-implemented application, the at least one interaction including user image data. For example, device 102 and/or user-health test function unit 140 may apply the at least one user-health test function to the at least one interaction between the at least one user and the at least one device-implemented application, the at least one interaction including user image data In one embodiment, the application 104 may be operable on the device 102 via a remote link such as network 192. A user's interaction with such an application 104 may generate user-health data 116 via a user input device 180, a user monitoring device 182, or a user interface 184. For example, the at least one device 102, user-health test function unit 140 and/or user-health test function selection module 138 can apply at least one eye movement test function to an interaction between a user 190 and a videocommunications application operable on device 102. The eye movement test function may act in conjunction with the videocommunications application on the device 102 to monitor the user's eye movements in the form of captured user image data. Other examples of appropriate contexts for user image data may include body movement test functions, pupil movement test functions, neglect test functions, and/or face pattern test functions.

The at least one device 102 and/or user-health test function unit 140 may apply a user-health test function in response to, for example, a user-health test function selection module 138 selecting the user-health test function at least partly based on a user's brain activity measurement data.

Operation 804 depicts applying the at least one user-health test function to the at least one interaction between the at least one user and the at least one device-implemented application, the at least one interaction including user pointing device manipulation data. For example, device 102 and/or user-health test function unit 140 may apply the at least one user-health test function to the at least one interaction between the at least one user and the at least one device-implemented application, the at least one interaction including user pointing device manipulation data. In one embodiment, at least one device 102, user-health test function unit 140 and/or user-health test function selection module 138 can apply at least one motor skill test function to an interaction between a user 190 and a game operable on the device 102. The motor skill test function may act in conjunction with the game to prompt the user to move a cursor within the game environment to activate objects, perhaps within a specified time. Other examples of appropriate contexts for pointing device manipulation data input may include body movement test functions, task sequencing functions, and/or reaction time test functions.

Examples of pointing devices include a computer mouse, a trackball, a touchscreen (e.g., on a personal digital assistant, on a laptop computer, or on a table surface computer), a joystick or other perspective-orienting device (e.g., a remote motion-sensor having accelerometer motion-detection capability), or other means of moving a cursor on a display or altering the perspective of an image on a display, including an image in a virtual environment. The at least one device 102 and/or user-health test function unit 140 may apply a user-health test function in response to, for example, a user-health test function selection module 138 selecting the user-health test function at least partly based on a user's brain activity measurement data.

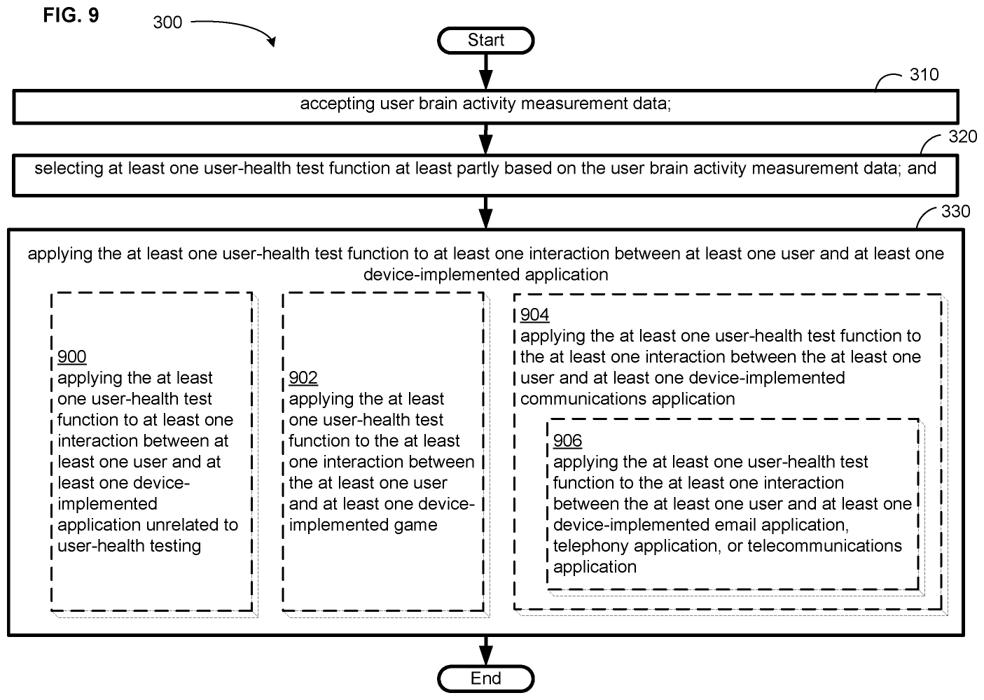

FIG. 9 illustrates alternative embodiments of the example operational flow 300 of FIG. 3. FIG. 9 illustrates example embodiments where the applying operation 330 may include at least one additional operation. Additional operations may include operation 900, 902, 904, and/or operation 906.

Operation 900 depicts applying the at least one user-health test function to at least one interaction between at least one user and at least one device-implemented application unrelated to user-health testing. For example, device 102 and/or user-health test function unit 140 may apply the at least one user-health test function to at least one interaction between at least one user and at least one device-implemented application unrelated to user-health testing. In one embodiment, user-health test function unit 140 may apply a selected user-health test function to an instant messaging exchange between a user 190 and another person, the instant messaging exchange having nothing at all to do with testing the user's health. Nevertheless, the exchange may provide user data that can be analyzed by the user-health test function in order to analyze one or more aspects of the user's health. For example, the user's ability to type and/or spell with a certain measurable proficiency may be analyzed by a user-health test function to examine, for example, aspects of the user's mental status, perhaps including attention, cognitive ability, motor coordination, or the like.

Operation 902 depicts applying the at least one user-health test function to the at least one interaction between the at least one user and at least one device-implemented game. For example, device 102 and/or user-health test function unit 140 may apply the at least one user-health test function to the at least one interaction between the at least one user and at least one device-implemented game. In one embodiment, at least one device 102 may have installed on it at least one game 106 that is operable on the at least one device 102. Such a game 106 may generate user-health data 116 via a user input device 180, a user monitoring device 182, or a user interface 184 as a result of an interaction with user 190. For example, the at least one device 102, user-health test function unit 140 and/or user-health test function selection module 138 can apply at least one calculation test function to an interaction between a user 190 and a game 106 operable on the device 102. The calculation test function may act in conjunction with the game to prompt the user to, for example, count, add, and/or subtract objects within the game environment. Other examples of a game 106 may include a cell phone game or other computer game such as, for example, solitaire, puzzle games, role-playing games, first-person shooting games, strategy games, sports games, racing games, adventure games, or the like. Such games may be played offline or through a network (e.g., online games).

For example, within a game situation, a user may be prompted to click on one or more targets within the normal gameplay parameters. User reaction time data may be collected once or many times for this task. The user reaction time data may be mapped to, for example, a mental status test function or a motor skill test function. User health data 116, including user reaction time test function output data, may indicate altered reaction time that are characteristic of a change in attention, such as loss of focus. The at least one device 102 and/or user-health test function selection module 138 may therefore select a user-health test function to test user attention, such as a test of the user's ability to accurately click a series of targets on a display within a period of time. Based on the outcome of this test, the device 102 and/or user-health test function unit can apply another reaction time test function, a motor skill test function, or other appropriate user-health test function.

Operation 904 depicts applying the at least one user-health test function to the at least one interaction between the at least one user and at least one device-implemented communications application. For example, device 102 and/or user-health test function unit 140 may apply the at least one user-health test function to the at least one interaction between the at least one user and at least one device-implemented communications application. In one embodiment, at least one communication application 108 may be resident on a server that is remote relative to the at least one device 102. Such an application 104 may generate user-health data 116 via a user input device 180, a user monitoring device 182 or a user interface 184. The at least one device 102 and/or user-health test function unit 140 can apply at least one user-health test function to the interaction of a user 190 with the at least one device-implemented communication application 108.

The at least one device 102, user-health test function unit 140, and/or user-health test function selection module 138 may apply a selected user-health test function to a communications application. For example, based on a selected user-health test function for analyzing user speech function, the at least one device 102, user-health test function unit 140, and/or user-health test function selection module 138 may apply a speech test function that monitors slurring of speech or stuttering during conversation of a user 190 on a cell phone.

Another example may include applying a user-health test function based on selection of a user-health test function for analyzing user-health data for a specific health diagnosis, such as dementia. In this example, the at least one device 102, user-health test function unit 140, and/or user-health test function selection module 138 may apply a memory test function that, for example, asks the user 190 to enter her mother's maiden name or other long term memory characteristic in the context of an email program as the communication application 108.

Examples of a communication application 108 may include various forms of one-way or two-way information transfer, typically to, from, between, or among devices. Some examples of communications applications include: an email program, a telephony application, a videocommunications function, an internet or other network messaging program, a cell phone communication application, or the like. Such a communication application may operate via text, voice, video, or other means of communication, combinations of these, or other means of communication.

Operation 906 depicts applying the at least one user-health test function to the at least one interaction between the at least one user and at least one device-implemented email application, telephony application, or telecommunications application. For example, device 102 and/or user-health test function unit 140 may apply the at least one user-health test function to the at least one interaction between the at least one user and at least one device-implemented email application, telephony application, or telecommunications application. In one embodiment, at least one application 104 may be resident, for example on a server that is remote relative to the at least one device 102. Such an application 104 may generate user-health data 116 via a user input device 180, a user monitoring device 182 or a user interface 184. The at least one device 102 and/or user-health test function unit 140 can apply at least one user-health test function to at least one device-implemented email application, telephony application, or telecommunications application whose primary function is different from symptom detection.

The at least one device 102, user-health test function unit 140, and/or user-health test function selection module 138 may apply a selected user-health test function to an email application, a telephony application, or a telecommunications application. For example, based on as selected user-health test function for analyzing an altered user face pattern, the at least one device 102, user-health test function unit 140, and/or user-health test function selection module 138 may apply a face pattern test function that monitors facial features and/facial feature movement during a video conference, web video chat, cell phone photograph or video, or the like.

Another example may include applying a user-health test function based on brain activity measurement data indicating a specific health diagnosis, such as depression. In this example, the at least one device 102, user-health test function unit 140, and/or user-health test function selection module 138 may apply a speech test function that, for example, monitors the abundance of a user's spontaneous speech during a time interval in the context of a cell phone application. Such a measure of spontaneous speech may provide an indication of depression.

Other examples of telecommunications applications include instant messaging, interactions of users with social networking internet sites (e.g., YouTube.com, MySpace.com, or the like), or other personal text, sound, or video messaging.

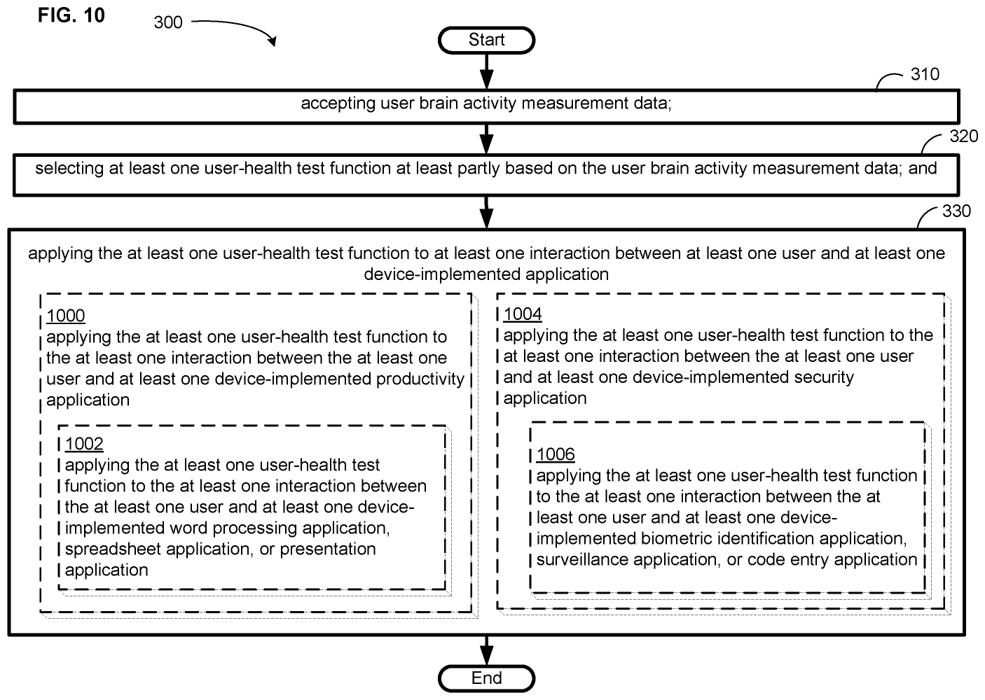

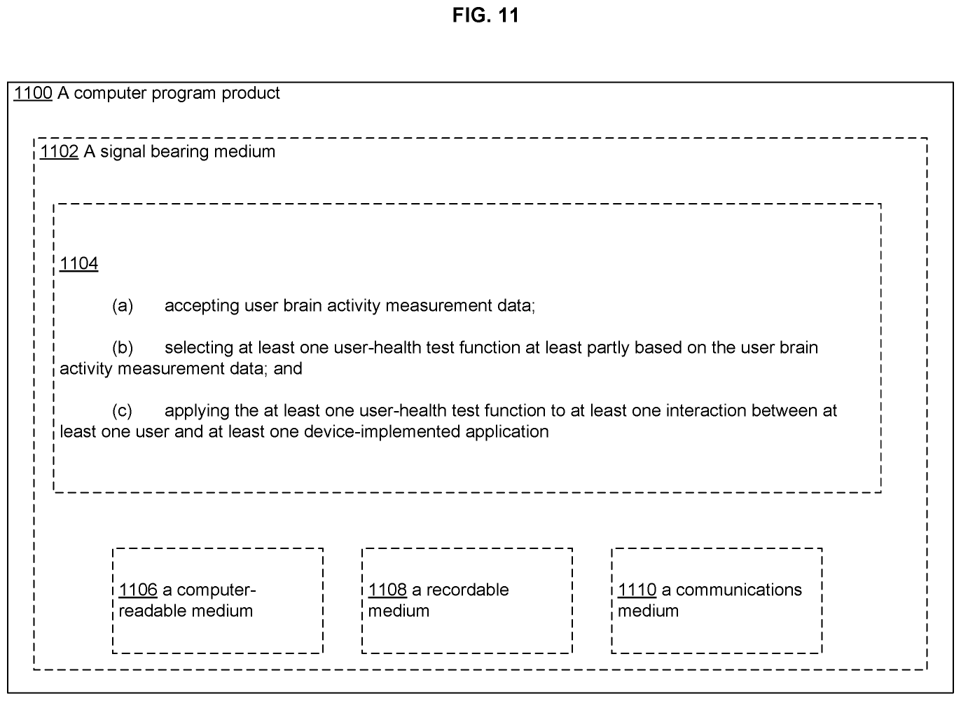

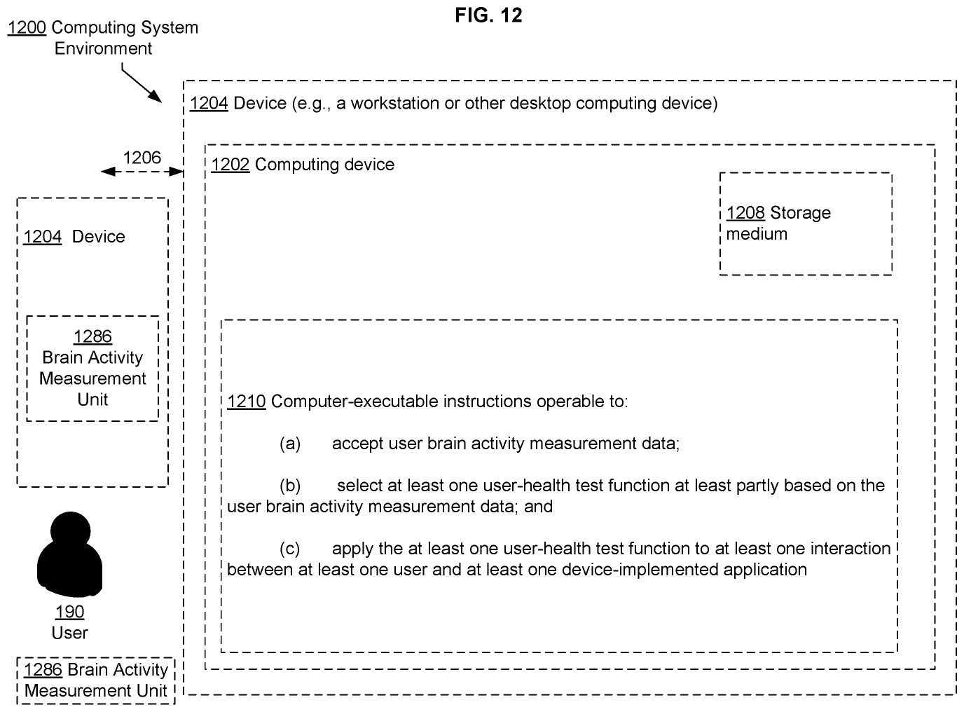

FIG. 10 illustrates alternative embodiments of the example operational flow 300 of FIG. 3. FIG. 10 illustrates example embodiments where the applying operation 330 may include at least one additional operation. Additional operations may include operation 1000, 1002, 1004, and/or operation 1006.

Operation 1000 depicts applying the at least one user-health test function to the at least one interaction between the at least one user and at least one device-implemented productivity application. For example, device 102 and/or user-health test function unit 140 may apply the at least one user-health test function to the at least one interaction between the at least one user and at least one device-implemented productivity application. In one embodiment, at least one device 102 may have installed on it at least one productivity application 112 that may generate user-health data 116 via a user input device 180 and/or a user monitoring device 182. For example, the at least one device 102, user-health test function unit 140 and/or user-health test function selection module 138 can apply at least one motor skill test function to an interaction between a user 190 and a productivity application 112 operable on the device 102. The motor skill test function may act in conjunction with the productivity application 112 to monitor the user's typing ability and/or pointing device manipulation ability within the parameters of the productivity application 112, or as an adjunct to actions within the productivity application 112. Examples of a productivity application 112 may include a word processing program, a spreadsheet program, other business software, or the like. Other examples of productivity applications may include a computer-aided drafting (“CAD”) application, an educational application, a project management application, a geographic information system (“GIS”) application, or the like.

For example, a user 190 may interact with a word processing application via a keyboard or other text input device. A device 102, user-health test function unit 140, and/or user-health test function selection module 138 may apply, for example, a mental status test function that monitors the frequency of use of the backspace key as a measure of a user's mental acuity, attention, and/or alertness.

Operation 1002 depicts applying the at least one user-health test function to the at least one interaction between the at least one user and at least one device-implemented word processing application, spreadsheet application, or presentation application. For example, device 102 and/or user-health test function unit 140 may apply the at least one user-health test function to the at least one interaction between the at least one user and at least one device-implemented word processing application, spreadsheet application, or presentation application. In one embodiment, at least one device 102 may have installed on it at least one word processing application, spreadsheet application, or presentation application that can generate user-health data 116 via a user input device 180 and/or a user monitoring device 182. For example, the at least one device 102, user-health test function unit 140 and/or user-health test function selection module 138 can apply at least one attention test function to an interaction between a user 190 and a word processing application, spreadsheet application, or presentation application operable on the device 102. The attention test function may act in conjunction with the word processing application, spreadsheet application, or presentation application to monitor the user's typing ability, calculation ability, reading ability, or pointing device manipulation ability, for example, within the parameters of the word processing application, spreadsheet application, or presentation application, or as an adjunct to actions within the word processing application, spreadsheet application, or presentation application.

For example, a user 190 may interact with a spreadsheet application via a keyboard or other text or number input device. A device 102, user-health test function unit 140, and/or user-health test function selection module 138 may apply, for example, a mental status test function that, for example, prompts the user to calculate a sum or construct an equation within the spreadsheet as a measure of the user's attention.

Operation 1004 depicts applying the at least one user-health test function to the at least one interaction between the at least one user and at least one device-implemented security application. For example, device 102 and/or user-health test function unit 140 may apply the at least one user-health test function to the at least one interaction between the at least one user and at least one device-implemented security application. In one embodiment, at least one device 102 may be operable within a system in which a security application 110 is operative. User interaction with the security application 110 may generate user-health data 116 via a user input device 180 and/or a user monitoring device 182. For example, the at least one device 102, user-health test function unit 140 and/or user-health test function selection module 138 can apply at least one pupil movement test function to an interaction between a user 190 and a security application 110. The pupil movement test function may act in conjunction with the security application 110 to monitor the user's pupillary reflex, for example, within the parameters of the security application 110, or as an adjunct to actions within the security application 110. Examples of a security application 110 may include a password entry program, a code entry system, a biometric identification application, a video monitoring system, other body-part recognition means such as ear geometry detection, pupil spacing detection, or the like.

Operation 1006 depicts applying the at least one user-health test function to the at least one interaction between the at least one user and at least one device-implemented biometric identification application, surveillance application, or code entry application. For example, device 102 and/or user-health test function unit 140 may apply the at least one user-health test function to the at least one interaction between the at least one user and at least one device-implemented biometric identification application, surveillance application, or code entry application. In one embodiment at least one device 102 may be operable within a system in which a security application 110 is operative. User interaction with the security application 110 may generate user-health data 116 via a user input device 180 and/or a user monitoring device 182. For example, the at least one device 102, user-health test function unit 140 and/or user-health test function selection module 138 can apply at least one pupil movement test function to an interaction between a user 190 and a security application 110 that authenticates a user's identity by matching retina patterns. The pupil movement test function may act in conjunction with the security application 110 to monitor the user's pupillary reflex, for example, within the parameters of the security application 110, or as an adjunct to actions within the security application 110.

Examples of a biometric identification application may include a fingerprint matching application, a facial feature matching application, a retina matching application, a voice pattern matching application, or the like. A biometric identification application includes identification functions, authentication functions, or the like, using personal characteristics as a reference against which identification or authentication may be measured. Examples of a surveillance application may include a video monitoring application, a voice detection application, or the like. Examples of a code entry application may include a mechanical or electronic lock requiring a code to unlock, a computerized security system requiring code entry for access or other functions, a software access feature requiring a code to access a program, or the like.

For example, a user 190 may interact with an eye imaging device in the course of using a retinal scanner. A device 102, user-health test function unit 140, and/or user-health test function selection module 138 may apply, for example, a pupil movement test function to the retinal scanner that, for example, detects pupil movement as a measure of the user's oculomotor nerve function, within the normal functioning of the retinal scanner.

In another embodiment, the at least one device 102 and/or user-health test function selection module 138 may, based on brain activity measurement data and optionally user-health data 116 indicative of a specific diagnosis, select a set of user-health test functions to apply. For example, as discussed above, a constellation of four kinds of altered user-health data 116 may indicate Gerstmann Syndrome; namely calculation deficit, right-left confusion, finger agnosia, and agraphia. Accordingly, the at least one device 102, user-health test function unit 140, and/or user-health test function selection module 138 may apply a group of user-health test functions to investigate the user's Gerstmann Syndrome profile, for example, if such symptoms are present in a user's medical history records. In this example, a system 100 may employ multiple user-health test functions in the context of multiple applications and/or devices. For example, a calculation test function may be applied in the context of a security application requiring a complex code for access to a program, object, or area; a neglect test function such as a right-left confusion test may be applied in the context of a security application that monitors user image data; and a speech test function or motor skill test function such as a finger agnosia test, agraphia, or writing test, may be applied in the context of an application 104 to complete the suite of test functions for Gerstmann's Syndrome.

FIG. 11 illustrates a partial view of an example computer program product 1100 that includes a computer program 1104 for executing a computer process on a computing device. An embodiment of the example computer program product 1100 is provided using a signal bearing medium 1102, and may include one or more instructions for accepting user brain activity measurement data; one or more instructions for selecting at least one user-health test function at least partly based on the user brain activity measurement data; and one or more instructions for applying the at least one user-health test function to at least one interaction between at least one user and at least one device-implemented application. The one or more instructions may be, for example, computer executable and/or logic-implemented instructions. In one implementation, the signal-bearing medium 1102 may include a computer-readable medium 1106. In one implementation, the signal bearing medium 1102 may include a recordable medium 1108. In one implementation, the signal bearing medium 1102 may include a communications medium 1110.

FIG. 12 illustrates an example system 1200 in which embodiments may be implemented. The system 1200 includes a computing system environment. The system 1200 also illustrates the user 190 using a device 1204, which is optionally shown as being in communication with a computing device 1202 by way of an optional coupling 1206. The optional coupling 1206 may represent a local, wide-area, or peer-to-peer network, or may represent a bus that is internal to a computing device (e.g., in example embodiments in which the computing device 1202 is contained in whole or in part within the device 1204). A storage medium 1208 may be any computer storage media. The system 1200 may also include a brain activity measurement unit 1286, which may be integrated into device 1204 or a separate unit.

The computing device 1202 includes computer-executable instructions 1210 that when executed on the computing device 1202 cause the computing device 1202 to (a) accept user brain activity measurement data; (b) select at least one user-health test function at least partly based on the user brain activity measurement data; and (c) apply the at least one user-health test function to at least one interaction between at least one user and at least one device-implemented application. As referenced above and as shown in FIG. 12, in some examples, the computing device 1202 may optionally be contained in whole or in part within the device 1204.

In FIG. 12, then, the system 1200 includes at least one computing device (e.g., 1202 and/or 1204). The computer-executable instructions 1210 may be executed on one or more of the at least one computing device. For example, the computing device 1202 may implement the computer-executable instructions 1210 and output a result to (and/or receive data from) the computing device 1204. Since the computing device 1202 may be wholly or partially contained within the computing device 1204, the device 1204 also may be said to execute some or all of the computer-executable instructions 1210, in order to be caused to perform or implement, for example, various ones of the techniques described herein, or other techniques.

The device 1204 may include, for example, a portable computing device, workstation, or desktop computing device. In another example embodiment, the computing device 1202 is operable to communicate with the device 1204 associated with the user 190 to receive information about the user 190 for performing data access and data processing and applying at least one user-health test function to at least one interaction between at least one user and at least one device-implemented application. Other examples of device 1204 may include one or more of a wearable computer, an implanted device, hearing aid or other personal health accessory device, a personal digital assistant (PDA), a personal entertainment device, a mobile phone, a laptop computer, a tablet personal computer, a networked computer, a computing system comprised of a cluster of processors, a computing system comprised of a cluster of servers.

Those skilled in the art will appreciate that the foregoing specific exemplary processes and/or devices and/or technologies are representative of more general processes and/or devices and/or technologies taught elsewhere herein, such as in the claims filed herewith and/or elsewhere in the present application.

Those having skill in the art will recognize that the state of the art has progressed to the point where there is little distinction left between hardware, software, and/or firmware implementations of aspects of systems; the use of hardware, software, and/or firmware is generally (but not always, in that in certain contexts the choice between hardware and software can become significant) a design choice representing cost vs. efficiency tradeoffs. Those having skill in the art will appreciate that there are various vehicles by which processes and/or systems and/or other technologies described herein can be effected (e.g., hardware, software, and/or firmware), and that the preferred vehicle will vary with the context in which the processes and/or systems and/or other technologies are deployed. For example, if an implementer determines that speed and accuracy are paramount, the implementer may opt for a mainly hardware and/or firmware vehicle; alternatively, if flexibility is paramount, the implementer may opt for a mainly software implementation; or, yet again alternatively, the implementer may opt for some combination of hardware, software, and/or firmware. Hence, there are several possible vehicles by which the processes and/or devices and/or other technologies described herein may be effected, none of which is inherently superior to the other in that any vehicle to be utilized is a choice dependent upon the context in which the vehicle will be deployed and the specific concerns (e.g., speed, flexibility, or predictability) of the implementer, any of which may vary. Those skilled in the art will recognize that optical aspects of implementations will typically employ optically-oriented hardware, software, and or firmware.

In some implementations described herein, logic and similar implementations may include software or other control structures suitable to operation. Electronic circuitry, for example, may manifest one or more paths of electrical current constructed and arranged to implement various logic functions as described herein. In some implementations, one or more media are configured to bear a device-detectable implementation if such media hold or transmit a special-purpose device instruction set operable to perform as described herein. In some variants, for example, this may manifest as an update or other modification of existing software or firmware, or of gate arrays or other programmable hardware, such as by performing a reception of or a transmission of one or more instructions in relation to one or more operations described herein. Alternatively or additionally, in some variants, an implementation may include special-purpose hardware, software, firmware components, and/or general-purpose components executing or otherwise invoking special-purpose components. Specifications or other implementations may be transmitted by one or more instances of tangible transmission media as described herein, optionally by packet transmission or otherwise by passing through distributed media at various times.

Alternatively or additionally, implementations may include executing a special-purpose instruction sequence or otherwise invoking circuitry for enabling, triggering, coordinating, requesting, or otherwise causing one or more occurrences of any functional operations described above. In some variants, operational or other logical descriptions herein may be expressed directly as source code and compiled or otherwise invoked as an executable instruction sequence. In some contexts, for example, C++ or other code sequences can be compiled directly or otherwise implemented in high-level descriptor languages (e.g., a logic-synthesizable language, a hardware description language, a hardware design simulation, and/or other such similar mode(s) of expression). Alternatively or additionally, some or all of the logical expression may be manifested as a Verilog-type hardware description or other circuitry model before physical implementation in hardware, especially for basic operations or timing-critical applications. Those skilled in the art will recognize how to obtain, configure, and optimize suitable transmission or computational elements, material supplies, actuators, or other common structures in light of these teachings.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In one embodiment, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disk, a hard disk drive, a Compact Disc (CD), a Digital Video Disk (DVD), a digital tape, a computer memory, etc.; and a transmission type medium such as a digital and/or an analog communication medium (e.g., a fiber optic cable, a waveguide, a wired communications link, a wireless communication link (e.g., transmitter, receiver, transmission logic, reception logic, etc.), etc.).

In a general sense, those skilled in the art will recognize that the various embodiments described herein can be implemented, individually and/or collectively, by various types of electro-mechanical systems having a wide range of electrical components such as hardware, software, firmware, and/or virtually any combination thereof; and a wide range of components that may impart mechanical force or motion such as rigid bodies, spring or torsional bodies, hydraulics, electro-magnetically actuated devices, and/or virtually any combination thereof. Consequently, as used herein “electro-mechanical system” includes, but is not limited to, electrical circuitry operably coupled with a transducer (e.g., an actuator, a motor, a piezoelectric crystal, a Micro Electro Mechanical System (MEMS), etc.), electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of memory (e.g., random access, flash, read only, etc.)), electrical circuitry forming a communications device (e.g., a modem, communications switch, optical-electrical equipment, etc.), and/or any non-electrical analog thereto, such as optical or other analogs. Those skilled in the art will also appreciate that examples of electro-mechanical systems include but are not limited to a variety of consumer electronics systems, medical devices, as well as other systems such as motorized transport systems, factory automation systems, security systems, and/or communication/computing systems. Those skilled in the art will recognize that electro-mechanical as used herein is not necessarily limited to a system that has both electrical and mechanical actuation except as context may dictate otherwise.

In a general sense, those skilled in the art will recognize that the various aspects described herein which can be implemented, individually and/or collectively, by a wide range of hardware, software, firmware, and/or any combination thereof can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of memory (e.g., random access, flash, read only, etc.)), and/or electrical circuitry forming a communications device (e.g., a modem, communications switch, optical-electrical equipment, etc.). Those having skill in the art will recognize that the subject matter described herein may be implemented in an analog or digital fashion or some combination thereof.

Those skilled in the art will recognize that at least a portion of the devices and/or processes described herein can be integrated into an image processing system. Those having skill in the art will recognize that a typical image processing system generally includes one or more of a system unit housing, a video display device, memory such as volatile or non-volatile memory, processors such as microprocessors or digital signal processors, computational entities such as operating systems, drivers, applications programs, one or more interaction devices (e.g., a touch pad, a touch screen, an antenna, etc.), control systems including feedback loops and control motors (e.g., feedback for sensing lens position and/or velocity; control motors for moving/distorting lenses to give desired focuses). An image processing system may be implemented utilizing suitable commercially available components, such as those typically found in digital still systems and/or digital motion systems.

Those skilled in the art will recognize that at least a portion of the devices and/or processes described herein can be integrated into a data processing system. Those having skill in the art will recognize that a data processing system generally includes one or more of a system unit housing, a video display device, memory such as volatile or non-volatile memory, processors such as microprocessors or digital signal processors, computational entities such as operating systems, drivers, graphical user interfaces, and applications programs, one or more interaction devices (e.g., a touch pad, a touch screen, an antenna, etc.), and/or control systems including feedback loops and control motors (e.g., feedback for sensing position and/or velocity; control motors for moving and/or adjusting components and/or quantities). A data processing system may be implemented utilizing suitable commercially available components, such as those typically found in data computing/communication and/or network computing/communication systems.

Those skilled in the art will recognize that at least a portion of the devices and/or processes described herein can be integrated into a mote system. Those having skill in the art will recognize that a typical mote system generally includes one or more memories such as volatile or non-volatile memories, processors such as microprocessors or digital signal processors, computational entities such as operating systems, user interfaces, drivers, sensors, actuators, applications programs, one or more interaction devices (e.g., an antenna USB ports, acoustic ports, etc.), control systems including feedback loops and control motors (e.g., feedback for sensing or estimating position and/or velocity; control motors for moving and/or adjusting components and/or quantities). A mote system may be implemented utilizing suitable components, such as those found in mote computing/communication systems. Specific examples of such components entail such as Intel Corporation's and/or Crossbow Corporation's mote components and supporting hardware, software, and/or firmware.

Those skilled in the art will recognize that it is common within the art to implement devices and/or processes and/or systems, and thereafter use engineering and/or other practices to integrate such implemented devices and/or processes and/or systems into more comprehensive devices and/or processes and/or systems. That is, at least a portion of the devices and/or processes and/or systems described herein can be integrated into other devices and/or processes and/or systems via a reasonable amount of experimentation. Those having skill in the art will recognize that examples of such other devices and/or processes and/or systems might include—as appropriate to context and application—all or part of devices and/or processes and/or systems of (a) an air conveyance (e.g., an airplane, rocket, helicopter, etc.), (b) a ground conveyance (e.g., a car, truck, locomotive, tank, armored personnel carrier, etc.), (c) a building (e.g., a home, warehouse, office, etc.), (d) an appliance (e.g., a refrigerator, a washing machine, a dryer, etc.), (e) a communications system (e.g., a networked system, a telephone system, a Voice over IP system, etc.), (f) a business entity (e.g., an Internet Service Provider (ISP) entity such as Comcast Cable, Qwest, Southwestern Bell, etc.), or (g) a wired/wireless services entity (e.g., Sprint, Cingular, Nextel, etc.), etc.

In certain cases, use of a system or method may occur in a territory even if components are located outside the territory. For example, in a distributed computing context, use of a distributed computing system may occur in a territory even though parts of the system may be located outside of the territory (e.g., relay, server, processor, signal-bearing medium, transmitting computer, receiving computer, etc. located outside the territory).

A sale of a system or method may likewise occur in a territory even if components of the system or method are located and/or used outside the territory.

Further, implementation of at least part of a system for performing a method in one territory does not preclude use of the system in another territory.

All of the above U.S. patents, U.S. patent application publications, U.S. patent applications, foreign patents, foreign patent applications and non-patent publications referred to in this specification and/or listed in any Application Data Sheet are incorporated herein by reference, to the extent not inconsistent herewith.

One skilled in the art will recognize that the herein described components (e.g., operations), devices, objects, and the discussion accompanying them are used as examples for the sake of conceptual clarity and that various configuration modifications are contemplated. Consequently, as used herein, the specific exemplars set forth and the accompanying discussion are intended to be representative of their more general classes. In general, use of any specific exemplar is intended to be representative of its class, and the non-inclusion of specific components (e.g., operations), devices, and objects should not be taken limiting.

Although user 190 is shown/described herein as a single illustrated figure, those skilled in the art will appreciate that user 190 may be representative of a human user, a robotic user (e.g., computational entity), and/or substantially any combination thereof (e.g., a user may be assisted by one or more robotic agents) unless context dictates otherwise. Those skilled in the art will appreciate that, in general, the same may be said of “sender” and/or other entity-oriented terms as such terms are used herein unless context dictates otherwise.

With respect to the use of substantially any plural and/or singular terms herein, those having skill in the art can translate from the plural to the singular and/or from the singular to the plural as is appropriate to the context and/or application. The various singular/plural permutations are not expressly set forth herein for sake of clarity.

The herein described subject matter sometimes illustrates different components contained within, or connected with, different other components. It is to be understood that such depicted architectures are merely exemplary, and that in fact many other architectures may be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled,” to each other to achieve the desired functionality, and any two components capable of being so associated can also be viewed as being “operably couplable,” to each other to achieve the desired functionality. Specific examples of operably couplable include but are not limited to physically mateable and/or physically interacting components, and/or wirelessly interactable, and/or wirelessly interacting components, and/or logically interacting, and/or logically interactable components.

In some instances, one or more components may be referred to herein as “configured to,” “configurable to,” “operable/operative to,” “adapted/adaptable,” “able to,” “conformable/conformed to,” etc. Those skilled in the art will recognize that “configured to” can generally encompass active-state components and/or inactive-state components and/or standby-state components, unless context requires otherwise.

While particular aspects of the present subject matter described herein have been shown and described, it will be apparent to those skilled in the art that, based upon the teachings herein, changes and modifications may be made without departing from the subject matter described herein and its broader aspects and, therefore, the appended claims are to encompass within their scope all such changes and modifications as are within the true spirit and scope of the subject matter described herein. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

With respect to the appended claims, those skilled in the art will appreciate that recited operations therein may generally be performed in any order. Also, although various operational flows are presented in a sequence(s), it should be understood that the various operations may be performed in other orders than those which are illustrated, or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

1-59. (canceled)

60. A method comprising:
providing instructions for performing at least one user-health test function in at least one memory associated with at least one computing device, the at least one user-health function including at least one speech test function;
executing at least one application on the at least one computing device, the at least one application including at least one of a game, a productivity application, a security application, a telephony application, a video communications application, a videoconferencing application, or a cell phone application;
electronically activating the at least one speech test function in response to a user command from at least one user to open the at least one application;
electronically selecting the at least one speech test function including at least obtaining brain activity measurement data associated with the at least one user, electronically analyzing the obtained brain activity measurement data, and selecting the at least one speech test function based at least partly on the analysis; and
electronically applying the selected at least one speech test function to at least one interaction of the at least one user with the at least one application including at least:
electronically generating a display of at least one picture;
prompting speech of the at least one user in response to the displayed at least one picture;
electronically accepting prompted user speech data of the at least one user during the at least one interaction with the at least one application;
electronically analyzing the prompted user speech data using at least speech recognition for at least one of syntactic complexity, fluency, phrase length, rate of speech, or grammar; and
determining at least one diagnosis regarding Alzheimer's disease associated with the at least one user based at least partly on the at least one of syntactic complexity, fluency, phrase length, rate of speech, or grammar.
